SEQUENCE LISTING (136 sequences)

SEQ ID NO: 1 - protein, 17 aa, Artificial Sequence
  Synthetic Construct
AKFLGQSTYIAGYHQVD

SEQ ID NO: 2 - protein, 17 aa, Artificial Sequence
  Synthetic Construct
FWSKMKPSEEYTTFYRD

SEQ ID NO: 3 - protein, 17 aa, Artificial Sequence
  Synthetic Construct
FDRSDYMSFHLDDNITI

SEQ ID NO: 4 - protein, 17 aa, Artificial Sequence
  Synthetic Construct
IRIETPYYKDTEDGKYF

SEQ ID NO: 5 - protein, 17 aa, Artificial Sequence
  Synthetic Construct
LGLLQAITRNSWVDSAF

SEQ ID NO: 6 - protein, 17 aa, Artificial Sequence
  Synthetic Construct
KHWEFMQFDIGYIYEKF

SEQ ID NO: 7 - protein, 17 aa, Artificial Sequence
  Synthetic Construct
NIPSNQHATEIQVDGYH

SEQ ID NO: 8 - protein, 17 aa, Artificial Sequence
  Synthetic Construct
ENFEYHLYDSMIGYEVH

SEQ ID NO: 9 - protein, 17 aa, Artificial Sequence
  Synthetic Construct
ERPDPATYFMPGRDDQY

SEQ ID NO: 10 - protein, 17 aa, Artificial Sequence
  Synthetic Construct
DNAPYYYREEWHKEFNK

SEQ ID NO: 11 - protein, 17 aa, Artificial Sequence
  Synthetic Construct
YHWDVQNTFYSMLMLPS

SEQ ID NO: 12 - protein, 17 aa, Artificial Sequence
  Synthetic  Construct
GDFSDYAPTLTQKASYG

SEQ ID NO: 13 - protein, 17 aa, Artificial Sequence
  Synthetic Construct
QYAFHNLDQNGTVFGNR

SEQ ID NO: 14 - protein, 17 aa, Artificial Sequence
  Synthetic Construct
YVTNMMINMNYSSLSYS

SEQ ID NO: 15 - protein, 17 aa, Artificial Sequence
  Synthetic Construct
HWVLSDGYREVYSYNSY

SEQ ID NO: 16 - protein, 17 aa, Artificial Sequence
  Synthetic Construct
AYHSQLYIDYKDTEWFY

SEQ ID NO: 17 - protein, 17 aa, Artificial Sequence
  Synthetic Construct
DHPYFVIWDRYKPVHTY

SEQ ID NO: 18 - protein, 17 aa, Artificial Sequence
  Synthetic Construct
HEHPPYLGMTAYELAQD

SEQ ID NO: 19 - protein, 17 aa, Artificial Sequence
  Synthetic Construct
GQWSGQGYWYDPFDNMK

SEQ ID NO: 20 - protein, 17 aa, Artificial Sequence
  Synthetic Construct
TTHFLKDRFESTNHDVY

SEQ ID NO: 21 - protein, 17 aa, Artificial Sequence
  Synthetic Construct
VEDPRVGHSLFQDANYY

SEQ ID NO: 22 - protein, 17 aa, Artificial Sequence
  Synthetic Construct
DPVQIFNTAEHSGPYIR

SEQ ID NO: 23 - protein, 17 aa, Artificial Sequence
  Synthetic Construct
HDHFRGGKFILSTQAIW

SEQ ID NO: 24 - protein, 17 aa, Artificial Sequence
  Synthetic Construct
NYPLPKYYYNWFEPRVW

SEQ ID NO: 25 - protein, 17 aa, Artificial Sequence
  Synthetic Construct
QYDDPDWQIHYKLEARG

SEQ ID NO: 26 - protein, 17 aa, Artificial Sequence
  Synthetic Construct
QDPYMDLHYDKNQIEQA

SEQ ID NO: 27 - protein, 17 aa, Artificial Sequence
  Synthetic Construct
RGPHNFEIAETDAQMIE

SEQ ID NO: 28 - protein, 17 aa, Artificial Sequence
  Synthetic Construct
DAYTNQDISEEEHMHRY

SEQ ID NO: 29 - protein, 17 aa, Artificial Sequence
  Synthetic Constuct
GALEWIYYAGPKPGYWE

SEQ ID NO: 30 - protein, 17 aa, Artificial Sequence
  Synthetic Construct
PHSTHQIFYKSYETDMA

SEQ ID NO: 31 - protein, 17 aa, Artificial Sequence
  Synthetic Construct
IYWGPMSTGHLPSQAQF

SEQ ID NO: 32 - protein, 17 aa, Artificial Sequence
  Synthetic Construct
YTHFWWLDKYMRYEVAT

SEQ ID NO: 33 - protein, 17 aa, Artificial Sequence
  Synthetic Construct
FLTEYYEYQNNLFHAFR

SEQ ID NO: 34 - protein, 17 aa, Artificial Sequence
  Synthetic Construct
SRGDAAAWGILFDANWK

SEQ ID NO: 35 - protein, 17 aa, Artificial Sequence
  Synthetic Construct
AMLGGMWAAYYPFPVPG

SEQ ID NO: 36 - protein, 17 aa, Artificial Sequence
  Synthetic Construct
YYWATGPEGPFRHPGAR

SEQ ID NO: 37 - protein, 17 aa, Artificial Sequence
  Synthetic Construct
DMEFTVFDIDMEKHYKY

SEQ ID NO: 38 - protein, 17 aa, Artificial Sequence
  Synthetic Construct
HYWDYQQLGFQGHLDHR

SEQ ID NO: 39 - protein, 17 aa, Artificial Sequence
  Synthetic Construct
DQHFVWGPTGRAPMNYG

SEQ ID NO: 40 - protein, 17 aa, Artificial Sequence
  Synthetic Construct
TNGFHIPFYSDFQSAAA

SEQ ID NO: 41 - protein, 17 aa, Artificial Sequence
  Synthetic Construct
MKYDVWKFYNGDDMRVS

SEQ ID NO: 42 - protein, 17 aa, Artificial Sequence
  Synthetic Construct
WYDNMYKTGFYRMYLLT

SEQ ID NO: 43 - protein, 17 aa, Artificial Sequence
  Synthetic Construct
EFQDYNHSDVNMSNHPY

SEQ ID NO: 44 - protein, 17 aa, Artificial Sequence
  Synthetic Construct
YIPEQQPHEADNLYKDA

SEQ ID NO: 45 - protein, 17 aa, Artificial Sequence
  Synthetic Construct
FYTLPHRPLYYYGYVAS

SEQ ID NO: 46 - protein, 17 aa, Artificial Sequence
  Synthetic Construct
HRWLEEANTEYMTMNSI

SEQ ID NO: 47 - protein, 17 aa, Artificial Sequence
  Synthetic Construct
GPINAYQKQDYSIEPEH

SEQ ID NO: 48 - protein, 17 aa, Artificial Sequence
  Synthetic Construct
AGDQYVRIDKSTRISNI

SEQ ID NO: 49 - protein, 17 aa, Artificial Sequence
  Synthetic Construct
SMKTVEADWYTYEPWWH

SEQ ID NO: 50 - protein, 17 aa, Artificial Sequence
  Synthetic Construct
NHIYIVQTAYGVTGETS

SEQ ID NO: 51 - protein, 17 aa, Artificial Sequence
  Synthetic Construct
MFAYHRAWPVWSSVLHV

SEQ ID NO: 52 - protein, 17 aa, Artificial Sequence
  Synthetic Construct
WPNPYFYHKKDTYWAHY

SEQ ID NO: 53 - protein, 17 aa, Artificial Sequence
  Synthetic Construct
NYDHLLGQYPIRNWWSL

SEQ ID NO: 54 - protein, 17 aa, Artificial Sequence
  Synthetic Construct
ESMDVVWPYGYKFTQYW

SEQ ID NO: 55 - protein, 17 aa, Artificial Sequence
  Synthetic Construct
THMEHDFHIPLEMYKYM

SEQ ID NO: 56 - protein, 17 aa, Artificial Sequence
  Synthetic Construct
VTPLEIIEQIREHLDIK

SEQ ID NO: 57 - protein, 17 aa, Artificial Sequence
  Synthetic Construct
LQKPFDYYMKEWQVDNE

SEQ ID NO: 58 - protein, 17 aa, Artificial Sequence
  Synthetic Construct
VMQRWPENHFLQTHYDD

SEQ ID NO: 59 - protein, 17 aa, Artificial Sequence
  Synthetic Construct
WWNWRSAYKEGDVAYPS

SEQ ID NO: 60 - protein, 17 aa, Artificial Sequence
  Synthetic Construct
GWVLDDWSSHHINYYIE

SEQ ID NO: 61 - protein, 17 aa, Artificial Sequence
  Synthetic Construct
FSPHYQYVGLFPYVKYI

SEQ ID NO: 62 - protein, 17 aa, Artificial Sequence
  Synthetic Construct
ANLVYMWGSAVHTSDPQ

SEQ ID NO: 63 - protein, 17 aa, Artificial Sequence
  Synthetic Construct
SPVMYSIANYKYQTMHL

SEQ ID NO: 64 - protein, 17 aa, Artificial Sequence
  Synthetic Construct
DQEISYLNSHTDLFVGR

SEQ ID NO: 65 - protein, 17 aa, Artificial Sequence
  Synthetic Construct
YEPTDVYLTYRKLATKD

SEQ ID NO: 66 - protein, 17 aa, Artificial Sequence
  Synthetic Construct
PVPQERTDLFTGAHRAL

SEQ ID NO: 67 - protein, 17 aa, Artificial Sequence
  Synthetic Construct
REVYHEIKSGRAIEIYM

SEQ ID NO: 68 - protein, 17 aa, Artificial Sequence
  Synthetic Construct
HDAIWYDWNPYPSKHES

SEQ ID NO: 69 - protein, 20 aa, Artificial Sequence
  Synthetic Construct
CSGAKFLGQSTYIAGYHQVD

SEQ ID NO: 70 - protein, 20 aa, Artificial Sequence
  Synthetic Construct
CSGFWSKMKPSEEYTTFYRD

SEQ ID NO: 71 - protein, 20 aa, Artificial Sequence
  Synthetic Construct
CSGFDRSDYMSFHLDDNITI

SEQ ID NO: 72 - protein, 20 aa, Artificial Sequence
  Synthetic Construct
CSGIRIETPYYKDTEDGKYF

SEQ ID NO: 73 - protein, 20 aa, Artificial Sequence
  Synthetic Construct
CSGLGLLQAITRNSWVDSAF

SEQ ID NO: 74 - protein, 20 aa, Artificial Sequence
  Synthetic Construct
CSGKHWEFMQFDIGYIYEKF

SEQ ID NO: 75 - protein, 20 aa, Artificial Sequence
  Synthetic Construct
CSGNIPSNQHATEIQVDGYH

SEQ ID NO: 76 - protein, 20 aa, Artificial Sequence
  Synthetic Construct
CSGENFEYHLYDSMIGYEVH

SEQ ID NO: 77 - protein, 20 aa, Artificial Sequence
  Synthetic Construct
CSGERPDPATYFMPGRDDQY

SEQ ID NO: 78 - protein, 20 aa, Artificial Sequence
  Synthetic Construct
CSGDNAPYYYREEWHKEFNK

SEQ ID NO: 79 - protein, 20 aa, Artificial Sequence
  Synthetic Construct
CSGYHWDVQNTFYSMLMLPS

SEQ ID NO: 80 - protein, 20 aa, Artificial Sequence
  Synthetic Construct
CSGGDFSDYAPTLTQKASYG

SEQ ID NO: 81 - protein, 20 aa, Artificial Sequence
  Synthetic Construct
CSGQYAFHNLDQNGTVFGNR

SEQ ID NO: 82 - protein, 20 aa, Artificial Sequence
  Synthetic Construct
CSGYVTNMMINMNYSSLSYS

SEQ ID NO: 83 - protein, 20 aa, Artificial Sequence
  Synthetic Construct
CSGHWVLSDGYREVYSYNSY

SEQ ID NO: 84 - protein, 20 aa, Artificial Sequence
  Synthetic Construct
CSGAYHSQLYIDYKDTEWFY

SEQ ID NO: 85 - protein, 20 aa, Artificial Sequence
  Synthetic Construct
CSGDHPYFVIWDRYKPVHTY

SEQ ID NO: 86 - protein, 20 aa, Artificial Sequence
  Synthetic Construct
CSGHEHPPYLGMTAYELAQD

SEQ ID NO: 87 - protein, 20 aa, Artificial Sequence
  Synthetic Construct
CSGGQWSGQGYWYDPFDNMK

SEQ ID NO: 88 - protein, 20 aa, Artificial Sequence
  Synthetic Construct
CSGTTHFLKDRFESTNHDVY

SEQ ID NO: 89 - protein, 20 aa, Artificial Sequence
  Synthetic Construct
CSGVEDPRVGHSLFQDANYY

SEQ ID NO: 90 - protein, 20 aa, Artificial Sequence
  Synthetic Construct
CSGDPVQIFNTAEHSGPYIR

SEQ ID NO: 91 - protein, 20 aa, Artificial Sequence
  Synthetic Construct
CSGHDHFRGGKFILSTQAIW

SEQ ID NO: 92 - protein, 20 aa, Artificial Sequence
  Synthetic Construct
CSGNYPLPKYYYNWFEPRVW

SEQ ID NO: 93 - protein, 20 aa, Artificial Sequence
  Synthetic Construct
CSGQYDDPDWQIHYKLEARG

SEQ ID NO: 94 - protein, 20 aa, Artificial Sequence
  Synthetic Construct
CSGQDPYMDLHYDKNQIEQA

SEQ ID NO: 95 - protein, 20 aa, Artificial Sequence
  Synthetic Construct
CSGRGPHNFEIAETDAQMIE

SEQ ID NO: 96 - protein, 20 aa, Artificial Sequence
  Synthetic Construct
CSGDAYTNQDISEEEHMHRY

SEQ ID NO: 97 - protein, 20 aa, Artificial Sequence
  Synthetic Construct
CSGGALEWIYYAGPKPGYWE

SEQ ID NO: 98 - protein, 20 aa, Artificial Sequence
  Synthetic Construct
CSGPHSTHQIFYKSYETDMA

SEQ ID NO: 99 - protein, 20 aa, Artificial Sequence
  Synthetic Construct
CSGIYWGPMSTGHLPSQAQF

SEQ ID NO: 100 - protein, 20 aa, Artificial Sequence
  Synthetic Construct
CSGYTHFWWLDKYMRYEVAT

SEQ ID NO: 101 - protein, 20 aa, Artificial Sequence
  Synthetic Construct
CSGFLTEYYEYQNNLFHAFR

SEQ ID NO: 102 - protein, 20 aa, Artificial Sequence
  Synthetic Construct
CSGSRGDAAAWGILFDANWK

SEQ ID NO: 103 - protein, 20 aa, Artificial Sequence
  Synthetic Construct
CSGAMLGGMWAAYYPFPVPG

SEQ ID NO: 104 - protein, 20 aa, Artificial Sequence
  Synthetic Construct
CSGYYWATGPEGPFRHPGAR

SEQ ID NO: 105 - protein, 20 aa, Artificial Sequence
  Synthetic Construct
CSGDMEFTVFDIDMEKHYKY

SEQ ID NO: 106 - protein, 20 aa, Artificial Sequence
  Synthetic Construct
CSGHYWDYQQLGFQGHLDHR

SEQ ID NO: 107 - protein, 20 aa, Artificial Sequence
  Synthetic Construct
CSGDQHFVWGPTGRAPMNYG

SEQ ID NO: 108 - protein, 20 aa, Artificial Sequence
  Synthetic Construct
CSGTNGFHIPFYSDFQSAAA

SEQ ID NO: 109 - protein, 20 aa, Artificial Sequence
  Synthetic Construct
CSGMKYDVWKFYNGDDMRVS

SEQ ID NO: 110 - protein, 20 aa, Artificial Sequence
  CSGWYDNMYKTGFYRMYLLT
CSGWYDNMYKTGFYRMYLLT

SEQ ID NO: 111 - protein, 20 aa, Artificial Sequence
  Synthetic Construct
CSGEFQDYNHSDVNMSNHPY

SEQ ID NO: 112 - protein, 20 aa, Artificial Sequence
  Synthetic Construct
CSGYIPEQQPHEADNLYKDA

SEQ ID NO: 113 - protein, 20 aa, Artificial Sequence
  Synthetic Construct
CSGFYTLPHRPLYYYGYVAS

SEQ ID NO: 114 - protein, 20 aa, Artificial Sequence
  Synthetic Construct
CSGHRWLEEANTEYMTMNSI

SEQ ID NO: 115 - protein, 20 aa, Artificial Sequence
  Synthetic Construct
CSGGPINAYQKQDYSIEPEH

SEQ ID NO: 116 - protein, 20 aa, Artificial Sequence
  Synthetic Construct
CSGAGDQYVRIDKSTRISNI

SEQ ID NO: 117 - protein, 20 aa, Artificial Sequence
  Synthetic Construct
CSGSMKTVEADWYTYEPWWH

SEQ ID NO: 118 - protein, 20 aa, Artificial Sequence
  Synthetic Construct
CSGNHIYIVQTAYGVTGETS

SEQ ID NO: 119 - protein, 20 aa, Artificial Sequence
  Synthetic Construct
CSGMFAYHRAWPVWSSVLHV

SEQ ID NO: 120 - protein, 20 aa, Artificial Sequence
  Synthetic Construct
CSGWPNPYFYHKKDTYWAHY

SEQ ID NO: 121 - protein, 20 aa, Artificial Sequence
  Synthetic Construct
CSGNYDHLLGQYPIRNWWSL

SEQ ID NO: 122 - protein, 20 aa, Artificial Sequence
  Synthetic Construct
CSGESMDVVWPYGYKFTQYW

SEQ ID NO: 123 - protein, 20 aa, Artificial Sequence
  Synthetic Construct
CSGTHMEHDFHIPLEMYKYM

SEQ ID NO: 124 - protein, 20 aa, Artificial Sequence
  Synthetic Construct
CSGVTPLEIIEQIREHLDIK

SEQ ID NO: 125 - protein, 20 aa, Artificial Sequence
  Synthetic Construct
CSGLQKPFDYYMKEWQVDNE

SEQ ID NO: 126 - protein, 20 aa, Artificial Sequence
  Synthetic Construct
CSGVMQRWPENHFLQTHYDD

SEQ ID NO: 127 - protein, 20 aa, Artificial Sequence
  Synthetic Construct
CSGWWNWRSAYKEGDVAYPS

SEQ ID NO: 128 - protein, 20 aa, Artificial Sequence
  Synthetic Construct
CSGGWVLDDWSSHHINYYIE

SEQ ID NO: 129 - protein, 20 aa, Artificial Sequence
  Synthetic Construct
CSGFSPHYQYVGLFPYVKYI

SEQ ID NO: 130 - protein, 20 aa, Artificial Sequence
  Synthetic Construct
CSGANLVYMWGSAVHTSDPQ

SEQ ID NO: 131 - protein, 20 aa, Artificial Sequence
  Synthetic Construct
CSGSPVMYSIANYKYQTMHL

SEQ ID NO: 132 - protein, 20 aa, Artificial Sequence
  Synthetic Construct
CSGDQEISYLNSHTDLFVGR

SEQ ID NO: 133 - protein, 20 aa, Artificial Sequence
  Synthetic Construct
CSGYEPTDVYLTYRKLATKD

SEQ ID NO: 134 - protein, 20 aa, Artificial Sequence
  Synthetic Construct
CSGPVPQERTDLFTGAHRAL

SEQ ID NO: 135 - protein, 20 aa, Artificial Sequence
  Synthetic Construct
CSGREVYHEIKSGRAIEIYM

SEQ ID NO: 136 - protein, 20 aa, Artificial Sequence
  Synthetic Construct
CSGHDAIWYDWNPYPSKHES

(19) United States
(12) Patent Application Publication
(10) Pub. No.: US 2017/0052200 A1
(43) Pub. Date: Feb. 23, 2017

(54) METHODS AND COMPOSITIONS FOR THE DIAGNOSIS AND TREATMENT OF KAWASAKI DISEASE

(71) Applicant: Momenta Pharmaceuticals, Inc., Cambridge, MA (US)

(72) Inventors: Dorota A. BULIK, Malden, MA (US); Jay DUFFNER, Shirley, MA (US); Leona E. LING, Winchester, MA (US); Hetal SARVAIYA, Foster City, CA (US); Carlos J. BOSQUES, Arlington, MA (US)

(21) Appl. No.: 15/308,114

(22) PCT Filed: May 1, 2015
(86) PCT No.: PCT/US15/28853

§ 371 Date: Nov. 1, 2016

Related U.S. Application Data
(60) Provisional application No. 62/067,123, filed on Oct. 22, 2014.
(60) Provisional application No. 61/987,907, filed on May 2, 2014.

(51) Int. Cl. G01N 33/68; A61K 39/395; A61K 31/727

(52) U.S. Cl. CPC G01N 33/6893; A61K 31/727; A61K 39/395; G01N 2800/328; G01N 2800/52; G01N 2800/50

(57) ABSTRACT

The present invention relates to the determination of levels or expression of particular biomarkers in biological samples which can be utilized to diagnose, prognose, and treat Kawasaki disease in subjects, and further to select subjects who would benefit from a Kawasaki disease therapy other than, or in addition to, IVIG treatment. Accordingly, the present invention encompasses methods and compositions that utilize these biomarkers for the diagnosis, prognosis, and treatment of Kawasaki disease.

### BACKGROUND

Kawasaki disease is an acute, systemic vasculitis predominantly affecting young children. Clinical symptoms of Kawasaki disease include persistent fever not managed by antipyretic medications and antibiotics, rash, conjunctival infection, edema and erythema of the extremities, and oropharyngeal erythema.

The disease may impact the systemic vasculature, but the coronary arteries and cardiac tissue are particularly susceptible to damage. Inflammation of the coronary arteries and surrounding cardiac tissue may be mild and reversible or may be extensive, leading to cardiac artery aneurysms (ballooning) and stenosis (narrowing) of the arteries. An estimated 25% of Kawasaki disease subjects develop cardiac artery aneurysms or stenosis. While many subjects show recovery of cardiac functions and no angiographic evidence of cardiac artery aneurysms or stenosis following recovery, there can be evidence of continued endothelial and vascular dysfunction even years later. Subjects with larger aneurysms are at higher risk for myocardial infarct (MI) and other cardiovascular events later in life.

There is no specific test available to diagnose Kawasaki disease. Diagnosis largely is a process of ruling out diseases that cause similar signs and symptoms (http://www.mayoclinic.org/diseases-conditions/kawasaki-disease/basics/tests-diagnosis/con-20024663, Mar. 3, 2014).

### SUMMARY OF THE INVENTION

Applicants have discovered that determination of levels or expression of particular biomarkers (e.g., protein levels, mRNA levels, glycan abundance, and/or the binding properties of IgG) in biological samples can be utilized to diagnose, prognose, and treat Kawasaki disease in subjects, and further to select subjects who would benefit from a Kawasaki disease therapy other than, or in addition to, IVIG treatment. Accordingly, the present invention encompasses methods and compositions that utilize these proteins for the diagnosis, prognosis, and treatment of Kawasaki disease.

In a first aspect, the invention features a method for diagnosing Kawasaki disease in a subject. This method includes the step of determining the level of one or more (e.g., two, three, four, five, six, seven, eight, nine, ten, twelve, fifteen, twenty, twenty-five, thirty, or more) proteins of Table 1 and/or Table 2 e.g., two, three, four, five, six, seven, eight, nine, ten, twelve, fifteen, twenty, twenty-five, thirty, or more and/or determining whether IgG in the sample binds to one or more (e.g., two, three, four, five, six, seven, eight, nine, ten, twelve, fifteen, twenty, twenty-five, thirty, forty, fifty, sixty, or more) peptides of Table 3.

TABLE 1
Selected Protein Biomarkers

| Protein Name | Protein Name |
|---|---|
| Alpha-1-antichymotrypsin(serpin | apolipoprotein F |
| peptidase inhibitor, clade A, | |
| member 3) | |
| lactate dehydrogenase C | zinc finger protein 578 |
| armadillo repeat containing 2 | xin actin-binding repeat containing 2 |
| unc-45 homolog A (C. elegans) | lipoprotein, Lp(a) |
| defensin, alpha 1 | armadillo repeat containing 10 |
| S100 calcium binding protein A9 | olfactory receptor, family 5, |
| | subfamily D, member 14 |
| solute carrier family 26 (anion | orosomucoid 1 |
| exchanger), member 3 | |
| zeta-chain (TCR) associated | Alpha-1-antitrypsin (serpin peptidase |
| protein kinase 70 kDa | inhibitor, clade A, member 1) |
| Zinc finger protein 106 homolog | nitric oxide synthase 2, inducible |
| | (Gene) |
| apolipoprotein B | carcinoembryonic antigen-related cell |
| | adhesion molecule 8 (Gene) |
| Zinc finger protein 161 homolog | interleukin 33 (Gene) |
| apolipoprotein M | interleukin 6 (interferon, beta 2) |
| | (Gene) |
| apolipoprotein C-II | matrix metallopeptidase 1 (interstitial |
| | collagenase) (Gene) |

TABLE 2
Selected Protein Biomarkers

| Protein Name | Protein Name |
|---|---|
| collagen, type VI, alpha 3 | Ig lambda-1 chain C regions (Mcg |
| | marker) |
| F-box protein 47 | BMP2 inducible kinase |
| collagen, type VI, alpha 1 | antithrombin III (serpin peptidase |
| | inhibitor, clade C, member 1) |
| androgen receptor | pregnancy-zone protein |
| tumor protein p53 binding protein 1 | inter-alpha-trypsin inhibitor heavy |
| | chain 1 |
| peptidase inhibitor 16 | Ig kappa chain V-II region |
| cadherin 13 | inter-alpha-trypsin inhibitor heavy |
| | chain 2 |
| tenascin XB | complement factor H |
| peroxisome proliferator-activated | Hepatocyte growth factor activator |
| receptor alpha | |
| collagen, type I, alpha 1 | attractin |
| Ig alpha-2 chain C region | kininogen 1 |
| (A2m marker) | |
| Ig heavy chain V-III region TIL | coagulation factor XIII, |
| | B polypeptide |
| Ig alpha-1 chain C region | complement factor B |
| olfactomedin 1 | complement component 1, r |
| | subcomponent |
| complement factor H | extracellular matrix protein 1 |
| prostaglandin D2 synthase 21 kDa | complement component 1, s |
| (brain) | subcomponent |
| sex hormone-binding globulin | complement component 6 |
| Ig kappa chain V-I region | vitamin D binding protein (group- |
| | specific component) |
| gelsolin | |

TABLE 3
Selected IgG Binding Peptides

| SEQ | | SEQ | |
|---|---|---|---|
| ID | | ID | |
| NO. | Peptide Sequence | NO. | Peptide Sequence |
| 1 | AKFLGQSTYIAGYHQVD | 35 | AMLGGMWAAYYPFPVPG |
| 2 | FWSKMKPSEEYTTFYRD | 36 | YYWATGPEGPFRHPGAR |
| 3 | FDRSDYMSFHLDDNITI | 37 | DMEFTVFDIDMEKHYKY |
| 4 | IRIETPYYKDTEDGKYF | 38 | HYWDYQQLGFQGHLDHR |
| 5 | LGLLQAITRNSWVDSAF | 39 | DQHFVWGPTGRAPMNYG |
| 6 | KHWEFMQFDIGYIYEKF | 40 | TNGFHIPFYSDFQSAAA |
| 7 | NIPSNQHATEIQVDGYH | 41 | MKYDVWKFYNGDDMRVS |
| 8 | ENFEYHLYDSMIGYEVH | 42 | WYDNMYKTGFYRMYLLT |
| 9 | ERPDPATYFMPGRDDQY | 43 | EFQDYNHSDVNMSNHPY |
| 10 | DNAPYYYREEWHKEFNK | 44 | YIPEQQPHEADNLYKDA |
| 11 | YHWDVQNTFYSMLMLPS | 45 | FYTLPHRPLYYYGYVAS |
| 12 | GDFSDYAPTLTQKASYG | 46 | HRWLEEANTEYMTMNSI |
| 13 | QYAFHNLDQNGTVFGNR | 47 | GPINAYQKQDYSIEPEH |
| 14 | YVTNMMINMNYSSLSYS | 48 | AGDQYVRIDKSTRISNI |
| 15 | HWVLSDGYREVYSYNSY | 49 | SMKTVEADWYTYEPWWH |
| 16 | AYHSQLYIDYKDTEWFY | 50 | NHIYIVQTAYGVTGETS |
| 17 | DHPYFVIWDRYKPVHTY | 51 | MFAYHRAWPVWSSVLHV |
| 18 | HEHPPYLGMTAYELAQD | 52 | WPNPYFYHKKDTYWAHY |
| 19 | GQWSGQGYWYDPFDNMK | 53 | NYDHLLGQYPIRNWWSL |
| 20 | TTHFLKDRFESTNHDVY | 54 | ESMDVVWPYGYKFTQYW |
| 21 | VEDPRVGHSLFQDANYY | 55 | THMEHDFHIPLEMYKYM |
| 22 | DPVQIFNTAEHSGPYIR | 56 | VTPLEIIEQIREHLDIK |
| 23 | HDHFRGGKFILSTQAIW | 57 | LQKPFDYYMKEWQVDNE |
| 24 | NYPLPKYYYNWFEPRVW | 58 | VMQRWPENHFLQTHYDD |
| 25 | QYDDPDWQIHYKLEARG | 59 | WWNWRSAYKEGDVAYPS |
| 26 | QDPYMDLHYDKNQIEQA | 60 | GWVLDDWSSHHINYYIE |
| 27 | RGPHNFEIAETDAQMIE | 61 | FSPHYQYVGLFPYVKYI |
| 28 | DAYTNQDISEEEHMHRY | 62 | ANLVYMWGSAVHTSDPQ |
| 29 | GALEWIYYAGPKPGYWE | 63 | SPVMYSIANYKYQTMHL |
| 30 | PHSTHQIFYKSYETDMA | 64 | DQEISYLNSHTDLFVGR |
| 31 | IYWGPMSTGHLPSQAQF | 65 | YEPTDVYLTYRKLATKD |
| 32 | YTHFWWLDKYMRYEVAT | 66 | PVPQERTDLFTGAHRAL |
| 33 | FLTEYYEYQNNLFHAFR | 67 | REVYHEIKSGRAIEIYM |
| 34 | SRGDAAAWGILFDANWK | 68 | HDAIWYDWNPYPSKHES |

According to this method, an increased level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) of at least one protein of Table 1, as compared to a reference (e.g., a control, such as a predetermined control value, or a sample from a subject that does not have Kawasaki disease), and/or a decreased level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less) of at least one protein of Table 2, as compared to a reference (e.g., a control, such as a predetermined control value, or a sample from a subject that does not have Kawasaki disease), and/or increased binding (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) of IgG in said sample to a peptide of Table 3, as compared to a reference (e.g., a control, such as a predetermined control value, or a sample from a subject that does not have Kawasaki disease) is indicative of the subject having Kawasaki disease.

In some embodiments, the method further includes the step of determining the level of one or more (e.g., two, three, four, five, six, seven, eight, nine, ten, twelve, fifteen, twenty, twenty-five, thirty, or more) additional biomarkers in the biological sample. In certain embodiments, the one or more additional biomarkers are a protein of Table 4, Table 5, Table 6, Table 7, Table 8, and/or Table 9; an mRNA of Table 10, Table 11, Table 12, and/or Table 13; and/or a glycan of Table 14, Table 15, Table 16, and/or Table 17.

TABLE 4
Selected Protein Biomarkers

L-lactate dehydrogenase
CD44 antigen
Ankyrin repeat domain-containing protein 26
Lysosome-associated membrane glycoprotein 2
Basal cell adhesion molecule
Multimerin-1
Transforming growth factor-beta-induced protein ig-h3
Methylcytosine dioxygenase TET2
Alpha-1-antichymotrypsin
Cystatin-C
Aftiphilin
Death domain containing 1
Xin actin-binding repeat containing 2
Metastasis associated 1 family, member 3

TABLE 5
Selected Protein Biomarkers

Plasminogen
Vitamin K-dependent protein C
Coagulation factor XI
Apolipoprotein F
HGF activator
Bone marrow stromal cell antigen 1
Mannan-binding lectin serine peptidase 2
HCG2014417, isoform CRA a
T-lymphoma invasion and metastasis-inducing protein 1

TABLE 6
Selected Protein Biomarkers

IgA
Hemoglobin subunit gamma-1
Hemoglobin subunit gamma-2
Protein S100-A9
Protein S100-A8
Leucine-rich alpha-2-glycoprotein
orosomucoid 1
orosomucoid 2
Actin, cytoplasmic 1
Haptoglobin-related protein
Cathepsin D
C-reactive protein
Xin actin-binding repeat containing 2
Beta-galactoside alpha-2,6-sialyltransferase 1
Complement component 9
Haptoglobin
Matrix metallopeptidase 9
Nidogen-1

TABLE 7
Selected Protein Biomarkers

Gelsolin
Kallistatin
Kininogen-1
Lumican
Alpha-2-HS-glycoprotein
butyrylcholinesterase
Coagulation factor IX
Protein C
Ig heavy chain V-III region BRO
Selenoprotein P
Cadherin-13
Myosin-8
Androgen receptor
Afamin
Biotinidase
Neural cell adhesion molecule L1-like protein
Coagulation factor X
Tenascin XB
Titin

TABLE 8
Selected Protein Biomarkers

Olfactory receptor, family 5, subfamily D, member 14
Orosomucoid 2
Polycystin (PKD) family receptor for egg jelly
Serpin peptidase inhibitor, clade A (alpha-1
antiproteinase, antitrypsin), member 3
Serpin peptidase inhibitor, clade A (alpha-1
antiproteinase, antitrypsin), member 1
Complement component 9
C-reactive protein, pentraxin related
Haptoglobin-related protein
Haptoglobin
Orosomucoid 1
Leucine-rich alpha-2-glycoprotein
Protein S100-A9
Lipopolysaccharide binding protein
Beta-galactoside alpha-2,6-sialyltransferase 1
Hemaglobin, alpha 1
Protein S100-A8
Hemoglobin, delta
Fc fragment of IgG, low affinity IIIa receptor (CD16a)
Hemaglobin, gamma G
Hemaglobin, gamma A
Hemaglobin, beta
Kinesin family member 20B
Lysozyme
Actin, alpha, cardiac muscle 1
Inter-alpha-trypsin inhibitor heavy chain 3
Inter-alpha-trypsin inhibitor heavy chain family, member 4
Actin, beta-like 2
Hemaglobin, epsilon 1
IgA
Protein phosphatase 6, regulatory subunit 2
Dedicator of cytokinesis 3
Cathepsin D
Centrosomal protein 290 kDa

TABLE 9
Selected Protein Biomarkers

Complement component 2
Complement factor H-related 3
Clusterin
Kallikrein B, plasma (Fletcher factor) 1
Macrophage stimulating 1 (hepatocyte growth factor-like)
Biotinidase
Peptidoglycan recognition protein 2
Fibulin 1
Kininogen 1
Gelsolin
Mannan-binding lectin serine peptidase 1 (C4/C2
activating component of Ra-reactive factor)
Alpha-1-microglobulin/bikunin precursor
Apolipoprotein H (beta-2-glycoprotein I)
Sex hormone-binding globulin
Collagen, type I, alpha 1
Inter-alpha-trypsin inhibitor heavy chain 2
Inter-alpha-trypsin inhibitor heavy chain 1
TBC1 domain family, member 8B (with GRAM domain)
Afamin
Heat shock 70 kDa protein 5 (glucose-regulated protein,
78 kDa)
BMP2 inducible kinase
Collagen, type VI, alpha 3
Kallistatin
Lumican
Cadherin 13
Neural cell adhesion molecule L1-like protein
Androgen receptor
Hepatocyte growth factor activator
Insulin-like growth factor binding protein,
acid labile subunit
Tenascin XB
Alpha-2-HS-glycoprotein
Glycosylphosphatidylinositol specific phospholipase D1
Retinol binding protein 4, plasma
Serpin peptidase inhibitor, clade C (antithrombin),
member 1
butyrylcholinesterase
Complement factor H
Centrosomal protein 70 kDa
Coagulation factor XIII, B polypeptide
Thrombospondin 4
Fibronectin 1
Insulin-like growth factor 2 (somatomedin A)
Protein C
Selenoprotein P, plasma 1
Neuropilin 1
Neural cell adhesion molecule 1
alpha-1-B glycoprotein
Coagulation factor XIII, A1 polypeptide
Extracellular matrix protein 1
Apolipoprotein C-1
Carboxypeptidase B2 (plasma)
Attractin
Zinc finger protein 217
Sex hormone-binding globulin
Pregnancy zone protein
Collagen, type XI, alpha 2
Selectin L
Ectonucleotide pyrophosphatase/
phosphodiesterase 2
Coagulation factor II (thrombin)
Apolipoprotein D
Plasmoginen
Insulin growth factor binding protein 3
Dehydrogenase/reductase (SDR family) member 11
Quiescin Q6 sulfhydryl oxidase 1

TABLE 10
Selected mRNA Biomarkers

CD80
Beta-glucuronidase

TABLE 11
Selected mRNA Biomarkers

carcinoembryonic antigen-related cell adhesion
molecule 8
S100 calcium binding protein A12
Versican

TABLE 12
Selected mRNA Biomarkers

Fc fragment of IgG, high affinity Ia, receptor (CD64)
S100 calcium binding protein A12
Matrix metallopeptidase 9
Tumor necrosis factor receptor superfamily, member 1A
Versican
Colony stimulating factor 2 receptor, beta, low affinity
(granulocyte-macrophage)
Interleukin 1, beta
Interleukin 1 receptor antagonist
S100 calcium binding protein A8
S100 calcium binding protein A11
Tumor necrosis factor receptor superfamily, member 1B
Tumor necrosis factor (ligand) superfamily, member 13b
Fc fragment of IgG, low affinity Iia receptor (CD32)
Chemokine (C-C motif) receptor 2
Actin, beta
Tumor necrosis factor

TABLE 13
Selected mRNA Biomarkers

Beta-glucuronidase
CD80
Killer cell lectin-like receptor subfamily G, member 1

TABLE 14
Selected IgG and IgG Glycan Biomarkers

GP38HA
GP23HA
GP41HA
GP28HA
GP37HA
GP42HA
GP43HA
GP43LA
GP44HA
GP44LA
GP9HA
GP9LA

TABLE 15
Selected IgG and IgG Glycan Biomarkers

GP6HA
GP4HA
GP26HA
GP11HA
GP5HA
GP7HA
GP2HA
GP12HA
IgG2_G0F_BGlcNAc
IgG2_BGlcNAc
GP3HA
IgG2_G1F_BGlcNAc
GP13HA
GP10HA
IgG3_4_A1
GP12LA
GP14HA
GP14LA
GP16LA
GP18HA
GP18LA
GP19LA
GP20HA
GP20LA
GP24LA
GP29LA
GP32HA
GP32LA
GP34HA
GP34LA
GP38LA
GP3LA

TABLE 16
Selected IgG and IgG Glycan Biomarkers

GP41LA
GP42LA
GP42HA
GP41HA
GP43LA
GP43HA
GP37HA
GP44HA
GP44LA
GP23HA
GP35HA
GP31HA
IgA_G1_S1

TABLE 17
Selected IgG and IgG Glycan Biomarkers

GP13HA
GP18HA
GP12LA
GP20LA
GP18LA
GP12HA
GP11HA
GP19HA
GP3HA
GP10HA
GP5HA
GP6HA
GP13LA
GP24LA
GP8HA
GP7HA
GP32LA
GP26HA
GP5LA
GP19LA
GP6LA
GP2HA
GP20HA

By this method, a subject can be further diagnosed for a predisposition to develop a secondary Kawasaki disease symptom, e.g., a cardiac artery aneurysm or stenosis. According to this method, an increased level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) of a protein of Table 4, Table 6, and/or Table 8, an mRNA of Table 10 or Table 12, and/or a glycan of Table 14 or Table 16, as compared to a reference (e.g., a control, such as a predetermined control value, or a sample from a subject that does not have Kawasaki disease or a subject that has Kawasaki disease and responded positively to IVIG treatment) and/or a decreased level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less) of a protein of Table 5, Table 7, and/or Table 9, an mRNA of Table 11 or Table 13, and/or a glycan of Table 15 or Table 17, as compared to a reference (e.g., a control, such as a predetermined control value, or a sample from a subject that does not have Kawasaki disease or a subject that has Kawasaki disease and responded positively to IVIG treatment) is indicative of said subject having a predisposition to develop a secondary Kawasaki disease symptom, e.g., a cardiac artery aneurysm or stenosis.

In a second aspect, the invention features a method for diagnosing whether a subject has a predisposition to develop cardiac artery aneurysms or stenosis (e.g., without an initial biomarker-based Kawasaki disease diagnosis). This method includes the step of determining the level of one or more (e.g., two, three, four, five, six, seven, eight, nine, ten, twelve, fifteen, twenty, twenty-five, thirty, or more) biomarkers of Table 4, Table 5, Table 6, Table 7, Table 8, Table 9, Table 10, Table 11, Table 12, Table 13, Table 14, Table 15, Table 16, and/or Table 17 in a biological sample obtained from the subject. According to this method, an increased level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) of at least one protein of Table 4, Table 6, and/or Table 8, an mRNA of Table 10 or Table 12, and/or a glycan of Table 14 or Table 16, as compared to a reference (e.g., a control, such as a predetermined control value, or a sample from a subject that does not have Kawasaki disease), and/or a decreased level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less) of at least one protein of Table 5, Table 7, and/or Table 9, an mRNA of Table 11 or Table 13, and/or a glycan of Table 15 or Table 17, as compared to a reference (e.g., a control, such as a predetermined control value, or a sample from a subject that does not have Kawasaki disease) is indicative of a subject having a predisposition to develop cardiac artery aneurysms or stenosis.

In a third aspect, the invention features a method for classifying a subject. Such classification includes predicting the response to a Kawasaki disease therapy in a subject, selecting a subject that may benefit from a Kawasaki disease therapy, selecting a subject who may benefit from IVIG therapy, or predicting the responsiveness of a subject to IVIG therapy. This method includes the step of determining the level of one or more (e.g., two, three, four, five, six, seven, eight, nine, ten, twelve, fifteen, twenty, twenty-five, thirty, or more) proteins of Table 1 and/or Table 2 in a biological sample obtained from the subject and/or determining whether IgG in the sample binds to one or more (e.g., two, three, four, five, six, seven, eight, nine, ten, twelve, fifteen, twenty, twenty-five, thirty, forty, fifty, sixty, or more) peptides of Table 3. According to this method, a subject is classified based on at least one or more of the proteins of Table 1 having an increased level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more), as compared to a reference (e.g., a control, such as a predetermined control value, or a sample from a subject that does not have Kawasaki disease), and/or at least one or more of the proteins of Table 2 having a decreased level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less), as compared to a reference (e.g., a control, such as a predetermined control value, or a sample from a subject that does not have Kawasaki disease), and/or increased binding (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) of IgG in said sample to a peptide of Table 3, as compared to a reference (e.g., a control, such as a predetermined control value, or a sample from a subject that does not have Kawasaki disease).

In some embodiments, the method further includes the step of determining the level of one or more (e.g., two, three, four, five, six, seven, eight, nine, ten, twelve, fifteen, twenty, twenty-five, thirty, or more) additional biomarkers in the biological sample. In certain embodiments, the one or more additional proteins are any protein of Table 4, Table 5, Table 6, Table 7, Table 8, and/or Table 9; an mRNA of Table 10, Table 11, Table 12, and/or Table 13; and/or a glycan of Table 14, Table 15, Table 16, and/or Table 17. By this step a subject can be further classified (for example, by determining the likelihood of a subject to develop cardiac artery aneurysms or stenosis, predicting the response to a Kawasaki disease therapy, selecting a subject that may benefit from a Kawasaki disease therapy other than, or in addition to, IVIG therapy, or predicting the responsiveness of a subject to IVIG therapy) based on one or more of the proteins of Table 4, Table 6, and/or Table 8, one or more mRNA of Table 10 or Table 12, and/or one or more glycan of Table 14 or Table 16 having an increased level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more), as compared to a reference (e.g., a control, such as a predetermined control value, or a sample from a subject that does not have Kawasaki disease or a subject that has Kawasaki disease and responded positively to IVIG treatment) and/or one or more of the proteins of Table 5, Table 7, and/or Table 9, one or more mRNA of Table 11 or Table 13, and/or one or more glycan of Table 15 or Table 17 having a decreased level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less), as compared to a reference (e.g., a control, such as a predetermined control value, or a sample from a subject that does not have Kawasaki disease or a subject that has Kawasaki disease and responded positively to IVIG treatment).

In a fourth aspect, the invention features a method for classifying a subject. Such classification includes determining the likelihood of a subject to develop cardiac artery aneurysms or stenosis, predicting the response to a Kawasaki disease therapy, selecting a subject that may benefit from a Kawasaki disease therapy other than, or in addition to, IVIG therapy, or predicting the responsiveness of a subject to IVIG therapy. The method includes: determining the level of one or more (e.g., two, three, four, five, six, seven, eight, nine, ten, twelve, fifteen, twenty, twenty-five, thirty, or more) biomarkers of Table 4, Table 5, Table 6, Table 7, Table 8, Table 9, Table 10, Table 11, Table 12, Table 13, Table 14, Table 15, Table 16, and/or Table 17 in a biological sample. According to this method, a subject is classified based on at least one or more of the Table 4, Table 6, and/or Table 8, one or more mRNA of Table 10 or Table 12, and/or one or more glycan of Table 14 or Table 16 having an increased level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more), as compared to a reference (e.g., a control, such as, a predetermined control value, or a sample from a subject that does not have Kawasaki disease or a subject that has Kawasaki disease and responded positively to IVIG treatment) and/or one or more Table 5, Table 7, and/or Table 9, one or more mRNA of Table 11 or Table 13, and/or one or more glycan of Table 15 or Table 17 having a decreased level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less), as compared to a reference (e.g., a control, such as a predetermined control value, or a sample from a subject that does not have Kawasaki disease or a subject that has Kawasaki disease and responded positively to IVIG treatment).

In a fifth aspect, the invention features a method for treating Kawasaki disease. The method includes: (a) determining the level of one or more (e.g., two, three, four, five, six, seven, eight, nine, ten, twelve, fifteen, twenty, twenty-five, thirty, or more) proteins from Table 1 or Table 2 in a biological sample obtained from the subject and/or determining whether IgG in the sample binds to one or more (e.g., two, three, four, five, six, seven, eight, nine, ten, twelve, fifteen, twenty, twenty-five, thirty, forty, fifty, sixty, or more) peptides of Table 3.; and (b) administering a Kawasaki disease therapy to the subject if the level of the one or more proteins is indicative that the subject may benefit from a Kawasaki disease therapy (e.g., administration of IVIG). In this method, an increased level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) of a protein of Table 1, as compared to a reference (e.g., a control, such as a predetermined control value, or a sample from a subject that does not have Kawasaki disease), is indicative that the subject may benefit from a Kawasaki disease therapy, and/or a decreased level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less) of a protein of Table 2, as compared to a reference (e.g., a control, such as a predetermined control value, or a sample from a subject that does not have Kawasaki disease), and/or increased binding (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) of IgG in said sample to a peptide of Table 3, as compared to a reference (e.g., a control, such as a predetermined control value, or a sample from a subject that does not have Kawasaki disease) is indicative that the subject may benefit from a Kawasaki disease therapy.

In some embodiments, the method further includes, prior to the determining step, the step of selecting a subject having a fever and one or more of: red eyes; a red swollen tongue; red skin on the palms on the hands and/or soles of the feet; peeling skin on the hands and/or feet; a rash on the main part of the body and/or in the genital area; and swollen lymph nodes and/or the step of obtaining a biological sample from said subject.

In other embodiments, the method further includes, between step (a) and step (b), the step of comparing the level of said one or more proteins to a reference (e.g., a predetermined control value) and/or comparing the binding of IgG in the sample to a reference (e.g., a predetermined control value).

In some embodiments, the method further includes (c) determining the level of one or more (e.g., two, three, four, five, six, seven, eight, nine, ten, twelve, fifteen, twenty, twenty-five, thirty, or more) biomarkers from Table 4, Table 5, Table 6, Table 7, Table 8, Table 9, Table 10, Table 11, Table 12, Table 13, Table 14, Table 15, Table 16, and/or Table 17 in a biological sample obtained from the subject; and (d) administering a Kawasaki disease therapy other than, or in addition to, IVIG therapy (e.g., a Kawasaki disease therapy including one or more anticoagulants such as enoxaparin and/or clopidogrel, an anti-inflammatory such as aspirin, and/or one or more immunosuppressant drugs such as infliximab, cyclosporine, and/or prednisone) to the subject if the level of the one or more biomarkers is indicative that the subject may benefit from a Kawasaki disease therapy other than, or in addition to, IVIG therapy. In this method, an increased level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) of a protein of Table 4, Table 6, and/or Table 8, an mRNA of Table 10 or Table 12, and/or a glycan of Table 14 or Table 16 as compared to a reference (e.g., a control, such as a predetermined control value, or a sample from a subject that does not have Kawasaki disease), is indicative that the subject may benefit from a Kawasaki disease therapy other than, or in addition to, IVIG therapy (e.g., a Kawasaki disease therapy including one or more anticoagulants such as enoxaparin and/or clopidogrel, an anti-inflammatory such as aspirin, and/or one or more immunosuppressant drugs such as infliximab, cyclosporine, and/or prednisone); and/or a decreased level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less) of a protein of Table 5, Table 7, and/or Table 9, an mRNA of Table 11 or Table 13, and/or a glycan of Table 15 or Table 17 as compared to a reference (e.g., a control, such as a predetermined control value, or a sample from a subject that does not have Kawasaki disease), is indicative that the subject may benefit from a Kawasaki disease therapy other than, or in addition to, IVIG therapy.

In a sixth aspect, the invention features a method of treating Kawasaki disease. The method includes: (a) determining the level of one or more (e.g., two, three, four, five, six, seven, eight, nine, ten, twelve, fifteen, twenty, twenty-five, thirty, or more) biomarkers from Table 4, Table 5, Table 6, Table 7, Table 8, Table 9, Table 10, Table 11, Table 12, Table 13, Table 14, Table 15, Table 16, and/or Table 17 in a biological sample obtained from the subject; and (b) administering a Kawasaki disease therapy other than, or in addition to, IVIG therapy (e.g., a Kawasaki disease therapy including one or more anticoagulants such as enoxaparin and/or clopidogrel, an anti-inflammatory such as aspirin, and/or one or more immunosuppressant drugs such as infliximab, cyclosporine, and/or prednisone) to the subject if the level of the one or more biomarkers is indicative that the subject may benefit from a Kawasaki disease therapy other than, or in addition to, IVIG therapy. In this method, an increased level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) of a protein of Table 4, Table 6, and/or Table 8, an mRNA of Table 10 or Table 12, and/or a glycan of Table 14 or Table 16 as compared to a reference (e.g., a control, such as a predetermined control value, or a sample from a subject that does not have Kawasaki disease), is indicative that the subject may benefit from a Kawasaki disease therapy other than, or in addition to, IVIG therapy (e.g., a Kawasaki disease therapy including one or more anticoagulants such as enoxaparin and/or clopidogrel, an anti-inflammatory such as aspirin, and/or one or more immunosuppressant drugs such as infliximab, cyclosporine, and/or prednisone); and/or a decreased level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less) of a protein of Table 5, Table 7, and/or Table 9, an mRNA of Table 11 or Table 13, and/or a glycan of Table 15 or Table 17 as compared to a reference (e.g., a control, such as a predetermined control value, or a sample from a subject that does not have Kawasaki disease), is indicative that the subject may benefit from a Kawasaki disease therapy other than, or in addition to, IVIG therapy.

Any of the methods herein that rely upon protein measurement can also be adapted for use with the measurement of mRNA levels for the protein. Accordingly, in a seventh aspect, the invention features a method for diagnosing Kawasaki disease in a subject, diagnosing whether a subject has a predisposition to develop cardiac artery aneurysms or stenosis, classifying a subject, or treating Kawasaki disease. This method includes the step of determining the level of mRNA encoding one or more (e.g., two, three, four, five, six, seven, eight, nine, ten, twelve, fifteen, twenty, twenty-five, thirty, or more) proteins of Tables 1-9 in a biological sample obtained from the subject. According to this method, increased level of mRNA (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) encoding a protein of Table 1, Table 4, Table 6, and/or Table 8 as compared to a reference (e.g., a control, such as a predetermined control value, or a sample from a subject that does not have Kawasaki disease), is a basis for classification of the subject and/or is indicative of the subject having Kawasaki disease, of the subject having a predisposition to develop cardiac artery aneurysms or stenosis, that the subject may benefit from a Kawasaki disease therapy, or that the subject may benefit from a Kawasaki disease therapy other than, or in addition to, IVIG therapy; and/or decreased mRNA level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less) of a protein of Table 2, Table 5, Table 7, and/or Table 9 as compared to a reference (e.g., a control, such as a predetermined control value, or a sample from a subject that does not have Kawasaki disease), is a basis for classification of the subject and/or is indicative of the subject having Kawasaki disease, of the subject having a predisposition to develop cardiac artery aneurysms or stenosis, that the subject may benefit from a Kawasaki disease therapy, or that the subject may benefit from a Kawasaki disease therapy other than, or in addition to, IVIG therapy.

In some embodiments, the method further includes the step of administering a Kawasaki disease therapy or a Kawasaki disease therapy other than, or in addition to, IVIG therapy if the mRNA level of the one or more proteins is indicative that the subject may benefit from a Kawasaki disease therapy or a Kawasaki disease therapy other than, or in addition to, IVIG therapy.

In other embodiments, the method further includes prior to determining the expression level, extracting mRNA from the biological sample and reverse transcribing the mRNA into cDNA to obtain a treated biological sample.

In certain embodiments, the mRNA level is determined by an amplification-based assay (e.g., PCR, quantitative PCR, or real-time quantitative PCR), amplification-free assay (e.g., Nanostring), microdroplet based assay, nanopore based assay, or bead based assays (e.g., Luminex, nanoparticles, Nanosphere).

Next generation sequencing methods may also be used with the methods of the invention. Next generation sequencing methods are sequencing technologies that parallelize the sequencing process, producing thousands or millions of sequences concurrently (see, for example, Hall, J. Exp. Biol. 209(Pt.9):1518-1525 (2007) for a review of next generation methods). Next generation sequencing methods include, but are not limited to, polony sequencing, 454 pyrosequencing, IIlumina (Solexa) sequencing, SOLiD sequencing, Ion Torrent semiconductor sequencing, DNA nanoball sequencing, Heliscope single molecule sequencing, single molecule real time sequencing, nanopore DNA sequencing (see, for example, Dela Torre et al. Nanotechnology, 23(38):385308, 2012), tunneling currents DNA sequencing (see, for example, Massimiliano, Nanotechnology, 24:342501, 2013), sequencing by hybridization (see, for example, Qin et al. PLoS One, 7(5):e35819, 2012), sequencing with mass spectrometry (see, for example, Edwards et al. Mutation Research, 573(1-2):3-12, 2005), microfluidic Sanger sequencing (see, for example, Kan et al. Electrophoresis, 25(21-22):3564-3588, 2004), microscopy-based sequencing (see, for example, Bell et al. Microscopy and microanalysis: the official journal of Microscopy Society of America, Microbeam Analysis Society, Microscopical Society of Canada, 18(5):1-5, 2012), and RNA polymerase sequencing (see, for example, Pareek et al. J. Applied Genetics, 52(4):413-415, 2011).

In an eighth aspect, the invention features a kit or device for selecting a subject that may benefit from a Kawasaki disease therapy. The kit or device includes a set of two or more (e.g., three, four, five, six, seven, eight, nine, ten, twelve, fifteen, twenty, twenty-five, thirty, or more) distinct binding agents, each of the binding agents being capable of specifically binding to at least one protein from Table 1 and/or Table 2, wherein each binding agent binds a different protein and/or one or more peptides from Table 3.

In a ninth aspect, the invention features a kit or device for selecting a subject that may benefit from a Kawasaki disease therapy. The kit or device includes a set of two or more (e.g., three, four, five, six, seven, eight, nine, ten, twelve, fifteen, twenty, twenty-five, thirty, or more) distinct reagents, each or the reagents being capable of detecting at least one mRNA that encodes a protein from Table 1 and/or Table 2, wherein each reagent detects a different mRNA that encodes a protein from Table 1 and/or Table 2.

Optionally the kit or device also includes instructions for use of the kit or device to determine the level of the proteins in a biological sample and/or instructions for use of the kit or device to determine the binding of IgG in the sample to the one or more peptides of Table 3.

In some embodiments of any of the foregoing kits or devices, the kit or device further includes a set of one or more (e.g., two, three, four, five, six, seven, eight, nine, ten, twelve, fifteen, twenty, twenty-five, thirty, or more) binding agents, each of the binding agents being capable of specifically binding to at least one protein, or the mRNA which encodes the protein, from Table 4, Table 5, Table 6, Table 7, Table 8, and/or Table 9; an mRNA or protein product of an mRNA of Table 10, Table 11, Table 12, and/or Table 13; and/or a glycan of Table 14, Table 15, Table 16, and/or 17.

In a tenth aspect, the invention features a kit or device for selecting a subject that may benefit from a Kawasaki disease therapy other than, or in addition to, IVIG therapy. The kit or device includes a set of two or more (e.g., two, three, four, five, six, seven, eight, nine, ten, twelve, fifteen, twenty, twenty-five, thirty, or more) binding agents, each of the binding agents being capable of specifically binding to at least one protein from Table 4, Table 5, Table 6, Table 7, Table 8, and/or Table 9; an mRNA or protein product of an m RNA of Table 10, Table 11, Table 12, and/or Table 13; and/or a glycan of Table 14, Table 15, Table 16, and/or 17 wherein each binding agent binds a different biomarker.

In an eleventh aspect, the invention features a kit or device for selecting a subject that may benefit from a Kawasaki disease therapy. The kit or device includes a set of two or more (e.g., three, four, five, six, seven, eight, nine, ten, twelve, fifteen, twenty, twenty-five, thirty, or more) distinct reagents, each or the reagents being capable of detecting at least one mRNA that encodes a protein from Table 4, Table 5, Table 6, Table 7, Table 8, and/or Table 9 wherein each reagent detects a different mRNA that encodes a protein from Table 4, Table 5, Table 6, Table 7, Table 8, and/or Table 9.

In a twelfth aspect, the invention features a method for diagnosing Kawasaki disease in a subject. The method includes determining the level of one or more proteins in a biological sample obtained from the subject with any of the foregoing kits or devices, wherein an increased level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) of a protein of Table 1, as compared to a reference (e.g., a control, such as a predetermined control value, or a sample from a subject that does not have Kawasaki disease), and/or a decreased level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less) of a protein of Table 2, as compared to a reference (e.g., a control, such as a predetermined control value, or a sample from a subject that does not have Kawasaki disease), and/or increased binding (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) of IgG in said sample to a peptide of Table 3, as compared to a reference (e.g., a control, such as a predetermined control value, or a sample from a subject that does not have Kawasaki disease) is indicative of the subject having Kawasaki disease.

In some embodiments, the method further includes the step of determining the level of one or more (e.g., two, three, four, five, six, seven, eight, nine, ten, twelve, fifteen, twenty, twenty-five, thirty, or more) additional biomarkers in the biological sample. In certain embodiments, the one or more additional biomarkers are any biomarker of Table 4, Table 5, Table 6, Table 7, Table 8, Table 9, Table 10, Table 11, Table 12, Table 13, Table 14, Table 15, Table 16, and/or Table 17. A subject can be further diagnosed with a predisposition to develop cardiac artery aneurysms or stenosis based on one or more of the proteins of Table 4, Table 6, and/or Table 8, an mRNA of Table 10 or Table 12, and/or a glycan of Table 14 or Table 16 having an increased level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more), as compared to a reference (e.g., a control, such as a predetermined control value, or a sample from a subject that does not have Kawasaki disease or a subject that has Kawasaki disease and responded positively to IVIG treatment) and/or one or more of the proteins of Table 5, Table 7, and/or Table 9, an mRNA of Table 11 or Table 13, and/or a glycan of Table 15 or Table 17 having a decreased level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less), as compared to a reference (e.g., a control, such as a predetermined control value, or a sample from a subject that does not have Kawasaki disease or a subject that has Kawasaki disease and responded positively to IVIG treatment) is indicative of the subject having a predisposition to develop cardiac artery aneurysms or stenosis.

In a thirteenth aspect, the invention features a method for diagnosing whether a subject has a predisposition to develop cardiac artery aneurysms or stenosis. This method includes the step of determining the level of one or more (e.g., two, three, four, five, six, seven, eight, nine, ten, twelve, fifteen, twenty, twenty-five, thirty, or more) biomarkers of Table 4, Table 5, Table 6, Table 7, Table 8, Table 9, Table 10, Table 11, Table 12, Table 13, Table 14, Table 15, Table 16, and/or Table 17 in a biological sample obtained from the subject with any of the foregoing kits or devices. According to this method, an increased level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) of at least one protein of Table 4, Table 6 and/or Table 8, and/or at least one glycan of Table 11, as compared to a reference (e.g., a control, such as a predetermined control value, or a sample from a subject that does not have Kawasaki disease), and/or a decreased level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less) of at least one protein of Table 5, Table 7, and/or Table 9, at least one mRNA of Table 11 or Table 13, and/or at least one glycan of Table 15 or Table 17, as compared to a reference (e.g., a control, such as a predetermined control value, or a sample from a subject that does not have Kawasaki disease) is indicative of a predisposition to develop cardiac artery aneurysms or stenosis in said subject.

In a fourteenth aspect, the invention features a method for treating Kawasaki disease in a subject. The method includes the steps of (a) determining the level of one or more proteins in a biological sample obtained from the subject and/or binding of IgG in the sample to one or more peptides with any of the foregoing kits or devices; and (b) administering a Kawasaki disease therapy to the subject if the level of the one or more proteins is indicative that the subject may benefit from a Kawasaki disease therapy (e.g., administration of IVIG). In this method, an increased level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) of a protein of Table 1, as compared to a reference (e.g., a control, such as a predetermined control value, or a sample from a subject that does not have Kawasaki disease), is indicative that the subject may benefit from a Kawasaki disease therapy, and/or a decreased level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less) of a protein of Table 2, as compared to a reference (e.g., a control, such as a predetermined control value, or a sample from a subject that does not have Kawasaki disease), and/or increased binding (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) of IgG in said sample to a peptide of Table 3, as compared to a reference (e.g., a control, such as a predetermined control value, or a sample from a subject that does not have Kawasaki disease) is indicative that the subject may benefit from a Kawasaki disease therapy.

In a fifteenth aspect, the invention features a further method for treating Kawasaki disease in a subject. This method includes the steps of (a) determining the level of one or more biomarkers in a biological sample obtained from the subject with any of the foregoing kits or devices; and (b) administering a therapy other than, or in addition to, IVIG therapy to the subject if the level of the one or more proteins is indicative that the subject may benefit from a Kawasaki disease therapy other than, or in addition to, IVIG therapy. In this method, an increased level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) of a protein of Table 4, Table 6, and/or Table 8, an mRNA of Table 10 or Table 12, and/or a glycan of Table 14 or Table 16 as compared to a reference (e.g., a control, such as a predetermined control value, or a sample from a subject that does not have Kawasaki disease), is indicative that the subject may benefit from a Kawasaki disease therapy other than, or in addition to, IVIG therapy (e.g., a Kawasaki disease therapy including one or more anticoagulants such as enoxaparin and/or clopidogrel, an anti-inflammatory such as aspirin, and/or one or more immunosuppressant drugs such as infliximab, cyclosporine, and/or prednisone); and/or a decreased level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less) of a protein of Table 5, Table 7, and/or Table 9, an mRNA of Table 11 or Table 13, and/or a glycan of Table 15 or Table 17 as compared to a reference (e.g., a control, such as a predetermined control value, or a sample from a subject that does not have Kawasaki disease), is indicative that the subject may benefit from a Kawasaki disease therapy other than, or in addition to, IVIG therapy.

In other embodiments of any of the foregoing methods, the Kawasaki disease therapy includes administration of IVIG to the subject (e.g., in high doses such as greater than 400 mg/kg, 500 mg/kg, 600 mg/kg, 700 mg/kg, 800 mg/kg, 900 mg/kg, 1 g/kg, 1.1 g/kg, 1.2 g/kg, 1.3 g/kg, 1.4 g/kg, 1.5 g/kg, 1.6 g/kg, 1.7 g/kg, 1.8 g/kg, 1.9 g/kg, 2.0 g/kg, 2.10 g/kg, 2.20 g/kg, 2.3 g/kg, 2.4 g/kg, 2.5 g/kg or more). In some embodiments, IVIG is administered to the subject between the fifth and ninth day after the appearance of symptoms. In other embodiments of any of the foregoing methods, the Kawasaki disease therapy includes administration of one or more anticoagulants (e.g., enoxaparin and/or clopidogrel or a pharmaceutically acceptable salt thereof) to the subject. In some embodiments of any of the foregoing methods, the Kawasaki disease therapy includes administration of an anti-inflammatory agent (e.g., aspirin). In other embodiments of any of the foregoing methods, the Kawasaki disease therapy includes administration of one or more immunosuppressant drugs (e.g., infliximab, cyclosporine, and/or prednisone).

In certain embodiments of any of the foregoing methods, the subject has one or more of: a fever; red eyes; a rash on the main part of the body and/or in the genital area; red, dry, cracked lips; a red, swollen tongue; swollen, red skin on the palms of the hands and/or soles of the feet; swollen lymph nodes; irritability; peeling of the skin on the hands and/or feet; joint pain; diarrhea; vomiting; and abdominal pain. For example, the subject may have a fever (e.g., a fever lasting more than four days) and one or more of: red eyes; a red swollen tongue; red skin on the palms of the hands and/or soles of the feet; peeling of the skin on the hands and/or feet; a rash on the main part of the body and/or in the genital area; and swollen lymph nodes.

In other embodiments of any of the foregoing methods, the subject exhibits the clinical symptoms of cardiac artery aneurysms and/or stenosis of the arteries. In yet other embodiments of any of the foregoing methods, the subject has not been diagnosed with cardiac artery aneurysms and/or stenosis of the arteries prior to determining the level of the one or more proteins. In certain embodiments of any of the foregoing methods, the subject has a white blood cell count and/or a C-reactive protein measurement that is not indicative of inflammation.

In some embodiments of any of the foregoing methods, the biological sample is obtained from the subject prior to the commencement of IVIG therapy. In other embodiments of any of the foregoing methods, the biological sample is obtained from the subject after commencement of IVIG therapy. In certain embodiments of any of the foregoing methods, the biological sample is obtained from the subject with 24 hours after commencement of IVIG therapy. In some embodiments of any of the foregoing methods, the biological sample is a tissue sample, whole blood, plasma, urine, saliva, pancreatic juice, bile, or serum sample. In certain embodiments of any of the foregoing methods, the biological sample is a plasma sample.

In some embodiments of any of the foregoing methods, the biological sample is processed prior to determining the level of the one or more the proteins, e.g., the biological sample is centrifuged, the biological sample is filtered, the biological sample is diluted, the biological sample is treated with reagents (e.g., digesting enzymes or reducing reagents), the biological sample is fractionated to remove more abundant proteins (e.g., proteins present at concentrations greater than 0.01 g/dL, greater than 0.02 g/dL, greater than 0.05 g/dL, greater than 0.1 g/dL, greater than 0.2 g/dL, greater than 0.5 g/dL, greater than 1.0 g/dL, greater than 2.0 g/dL, greater than 3.0 g/dL), such as, albumins, globulins (e.g., haptoglobulin, alpha2-macroglobulin, IgG, IgA, and IgM), alpha1-acid glycoprotein, apolipoprotein AI, apolipoprotein AII, complement C3, transthyretin, antitrypsin, transferrin, and fibrinogen and/or enrich for less abundant proteins, such as, any protein from Tables 1, 2, 4, or 5. In some embodiments, the biological sample is subjected to centrifugation to remove red blood cells. In certain embodiments, the biological sample is filtered (e.g., spin filtered). In some embodiments, the biological sample is diluted. In other embodiments, the biological sample is subjected to cold alcohol fractionation. In certain embodiments, the biological sample is subjected to chromatographic separation (e.g., using an immunoaffinity-based column). In some embodiments, the biological sample is concentrated. In other embodiments, the biological sample is buffer exchanged. In certain embodiments, the biological sample is treated with a digesting enzyme (e.g., trypsin).

In other embodiments any of the foregoing methods further include contacting the biological sample with one or more binding agents capable of specifically binding to the one or more proteins, one or more peptides of Table 3, one or more mRNAs of Table 10, Table 11, Table 12, and/or Table 13, and/or one or more glycans of Table 14, Table 15, Table 16, and/or Table 17.

In any of the aspects and embodiments described herein, the protein level and/or binding of IgG in the sample is determined by one or more of a hybridization assay, an immunoassay, liquid chromatography, mass spectrometry, and/or fluorescence in situ hybridization assay (e.g., Northern analysis, ELISA, immunohistochemical analysis, microarray, chip, microfluidic chip, sequencing, or Western blotting).

In certain embodiments of any of the foregoing methods, the subject is less than 18 years old (e.g., less than 17 years old, less than 16 years old, less than 15 years old, less than 14 years old, less than 13 years old, less than 12 years old, less than 11 years old, less than 10 years old, less than 9 years old, less than 8 years old, less than 7 years old, less than 6 years old, less than 5 years old, less than 4 years old, less than 3 years old, less than 2 years old, less than 1 year old, less than 6 months old). In some embodiments of any of the foregoing methods, the subject is Asian (e.g., Japanese or Korean) or Afro-Caribbean.

In other embodiments of any of the foregoing methods, the level of the one or more proteins and/or binding of IgG in the sample is determined at least twice within 365 days (e.g., twice within 180 days, within 90 days, within 60 days, within 30 days, within 14 days, within 7 days). In certain embodiments, the level of the one or more proteins and/or binding of IgG in the sample is determined at least once prior to the commencement of IVIG therapy and at least once after commencement of IVIG therapy.

In certain embodiments any of the foregoing kits or devices also include instructions for use of the kit or device to determine the level of the proteins in a biological sample, the binding of IgG to a peptide of Table 3, the expression level of an mRNA of Table 10, Table 11, Table 12, and/or Table 13, and/or the abundance of a glycan of Table 14, Table 15, Table 16, and/or Table 17.

In other embodiments of any of the foregoing methods, the method further includes the step of recording the result in a print or computer readable media. In other embodiments, the method further includes the step of informing (e.g., providing the results of the determining step on printable media) the subject that he or she has Kawasaki disease, may benefit from a Kawasaki disease therapy, may benefit from IVIG therapy, may have an increased likelihood to develop cardiac artery aneurysms and/or stenosis, may have a predisposition to develop cardiac artery aneurysms and/or stenosis, may benefit from a therapy other than, or in addition to, IVIG therapy, or may benefit from therapy that includes one or more anticoagulants, an anti-inflammatory agent, and/or one or more immunosuppressant drugs.

In some embodiments of any of the aspects described herein, the binding agent is an antibody. In other embodiments of any of the aspects described herein, one or more of the binding agents and/or peptides of Table 3 are provided on a solid support (e.g., as a microarray). In any of the aspects and embodiments described herein, the one or more proteins, one or more peptides of Table 3, one or more mRNAs of Table 10, Table 11, Table 12, and/or Table 13, one or more glycans of Table 14, Table 15, Table 16 and/or Table 17, and/or set of binding agents and/or peptides include or consist of any combination described herein. In any of the aspects and embodiments described herein, the one or more peptides of Table 3 may be attached to a solid support by a linker (e.g., an N-terminal or C-terminal cysteine, or an N-terminal or C-terminal cysteine-serine-glycine group).

Also provided herein are methods of monitoring a subject with Kawasaki disease. The diagnostic kits and methods disclosed herein can be used to determine an optimal treatment plan for a subject or to determine the efficacy of a treatment plan for a subject. For example, the subject can be treated for Kawasaki disease and the prognosis of the disease can be determined by the diagnostic kits and methods disclosed herein. In particular embodiments, a diagnostic kit or method is used to determine if a subject has Kawasaki disease. A diagnostic kit or method can include a screen for protein level and/or IgG binding profiles by any useful detection method (e.g., unlabeled, fluorescence, radiation, or chemiluminescence). A diagnostic test can further include one or more binding agents (e.g., one or more of probes, primers, peptides, small molecules, aptamers, or antibodies) to detect the level of these proteins or mRNAs encoding these proteins. In certain embodiments, the diagnostic kit includes the use of one or more proteins associated with Kawasaki disease and/or one or more peptides of Table 3 in a diagnostic platform, which can be optionally automated.

Also provided herein are general strategies to develop diagnostic tests which can be used to diagnose Kawasaki disease based on the level of proteins, binding of IgG to one or more peptides of Table 3, mRNAs of Table 10, Table 11, Table 12, and/or Table 13, one or more glycans of Table 14, Table 15, Table 16 and/or Table 17 disclosed herein. These strategies can be used to develop tests that use one or more of these proteins, peptides, mRNAs, and/or glycans, any combination of one or more of these proteins, peptides, mRNAs and/or glycans, one or more of these proteins, peptides, mRNAs, and/or glycans in combination with any other biomarkers found to be associated with Kawasaki disease, and/or one or more of these proteins, peptides, mRNAs, and/or glycans in combination with one or more reference biomarkers not associated with Kawasaki disease.

Also provided herein are methods of determining the likelihood of a subject to develop cardiac artery aneurysms or stenosis. Accordingly, the invention also includes methods of diagnosing a subject that would benefit from a therapy other than or in addition to, IVIG therapy by performing any of the methods or using any of the compositions or kits described herein.

Other features and advantages of the invention will be apparent from the following description and the claims.

### DEFINITIONS

As used herein, the term “about” means ±10% of the recited value.

The term “array” or “microarray,” as used herein refers to an ordered arrangement of hybridizable array elements, preferably protein probes (e.g., antibodies), on a substrate. The substrate can be a solid substrate, such as a glass slide, beads, or microfluidic chip, or a semi-solid substrate, such as nitrocellulose membrane.

The term “Afro-Caribbean” refers to a person of Caribbean descent (i.e., is from or has an ancestor from the Caribbean Region, as classified by the United Nations Department of Economic and Social Affairs) and has an ancestor that emigrated from Africa to the Caribbean Region in the period since 1492.

The term “anticoagulant” refers to a drug that works to prevent the coagulation of blood, such as coumarins, thienopyridines (e.g., clopidogrel), heparin, low molecular weight heparin (e.g., enoxaparin), inhibitors of factor Xa, or thrombin inhibitors.

The term “anti-inflammatory agent” refers to a drug that reduces inflammation in a subject, e.g., non-steroidal anti-inflammatory drugs (NSAIDs) such as aspirin, ibuprofen, and naproxen.

The term “Asian” refers to a person of Asian descent (i.e., is from or has an ancestor from the Eastern Asia or Southeastern Asia Regions, as classified by the United Nations Department of Economic and Social Affairs). For example, a person from, or having an ancestor from, Japan (i.e., someone who is Japanese); or a person from, or having an ancestor from, Korea (i.e., someone who is Korean) are Asian.

By a “binding agent” is meant any compound (e.g., a probe, primer, protein, small molecule, aptamer, or antibody) capable of specifically binding a target. By “specifically binds” is meant binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a binding agent is competitively inhibited by excess unlabeled target. The term “specific binding,” “specifically binding,” or “specifically binds to” a particular protein as used herein can be exhibited, for example, by a molecule having a K_{D} for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower, or a K_{D} in the range of 10⁻⁴ M to 10⁻¹² M or 10⁻⁶ M to 10⁻¹⁰ M or 10⁻⁷ M to 10⁻⁹ M. As will be appreciated by the skilled artisan, affinity and K_{D} values are inversely related. A high affinity for a target is measured by a low K_{D} value. In one embodiment, the term “specific binding” refers to binding where a binding agent binds to a particular protein, mRNA, or glycan without substantially binding to any other protein, mRNA, or glycan.

By “biological sample” or “sample” is meant a fluid or solid sample from a subject. Biological samples may include cells; nucleic acid, protein, or membrane extracts of cells; or blood or biological fluids including (e.g., plasma, serum, saliva, urine, bile). Solid biological samples include samples taken from feces, the rectum, central nervous system, bone, breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, and the thymus. Fluid biological samples include samples taken from the blood, serum, plasma, pancreatic fluid, CSF, semen, prostate fluid, seminal fluid, urine, saliva, sputum, mucus, bone marrow, lymph, and tears. Samples may be obtained by standard methods including, e.g., venous puncture and surgical biopsy. In certain embodiments, the biological sample is a blood, plasma, or serum sample.

By “classifying a subject” is meant predicting a response to a Kawasaki disease therapy by a subject; selecting a subject that may benefit from a Kawasaki disease therapy; selecting a subject who may benefit from IVIG therapy; predicting the responsiveness of a subject to IVIG therapy; determining the likelihood of a subject to develop cardiac artery aneurysms or stenosis; or selecting a subject that may benefit from a Kawasaki disease therapy other than, or in addition to, IVIG therapy.

By “diagnosing” is meant identifying a molecular or pathological state, disease or condition, such as the identification of Kawasaki disease or cardiac artery aneurysm and/or stenosis, or to refer to identification of a subject having Kawasaki disease who may benefit from a particular treatment regimen.

By “determining the level of a protein, mRNA, or glycan” is meant the detection of a protein, mRNA, or glycan by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA and glycan levels are known in the art. Exemplary methods are provided herein.

By “determining the binding of IgG” is meant the detection of binding of IgG in a sample (e.g., a plasma sample) to a binding agent (e.g., a peptide of Table 3) by methods known in the art. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third party laboratory that directly acquired the physical entity or value). Methods to measure binding generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), flow cytometry, peptide arrays, protein arrays and microarrays.

The term “immunosuppressant drug,” as used herein refers to a drug that inhibits or prevents activity of the immune system including glucocorticoids such as prednisone, cytostatics such as methotrexate, antibodies such as infliximab, drugs acting on immunophilins such as cyclosporine.

By “informing a subject” is meant providing the subject or the parent or legal guardian of the subject the results of the determining step and/or analysis of the results verbally and/or on printable media.

The term “IVIG” as used herein refers to intravenous immunoglobulin, a blood product containing pooled, polyvalent IgG extracted from the plasma of over one thousand blood donors. The term “IVIG therapy” refers to a treatment including the administration of IVIG to a subject, e.g., in high doses, such as, 2 g/kg.

By “Kawasaki disease therapy” is meant any therapy in the art for the treatment of Kawasaki disease, such as, therapeutic agents or modalities for Kawasaki disease. Common treatments for Kawasaki disease include administration of IVIG (i.e., IVIG therapy); salicylates (e.g., aspirin); corticosteroids (e.g., prednisone); IL-1 receptor antagonists; anticoagulants (e.g., enoxaparin and/or clopidogrel); anti-TNF agents (e.g., infliximab); or any combination thereof.

The terms “kit or device,” as used herein, refer to a set of articles and/or equipment, such as reagents, instruments, and systems, intended for use in diagnosis or prognosis of disease or other conditions, including determination of the state of health, in order to cure, mitigate, treat, or prevent disease or its sequelae. The kits and devices of the invention are intended for use in the collection, preparation, and/or examination of biological samples taken from the subject. For example, the kits and devices of the invention may be used for biochemical estimation or the qualitative detection of a protein. The kits and devices of the invention may include general purpose reagents and analyte specific reagents. A “general purpose reagent” refers to a chemical reagent that has general laboratory application, used to collect, prepare, and/or examine specimens from the human body for diagnostic purposes, and is not labeled or otherwise intended for a specific diagnostic application. An “analyte specific reagent” refers to antibodies, both polyclonal and monoclonal, specific receptor proteins, ligands, nucleic acids, and other binding agents which, through specific binding or chemical reaction with substances in a biological sample, are intended for use in a diagnostic application for identification and quantification of an individual chemical substance or ligand in biological samples. The kits and devices of the invention may include a label which states the name of the kit or device, the intended use or uses of the device (e.g., the diagnosis of Kawasaki disease), a statement of warnings or precautions for users of any hazardous substances contained in the kit or device and any other warnings appropriate to user hazards, the established name of the reagents, quantity, proportion, or concentration of all active ingredients and for reagents derived from biological activity, the source and measure of its activity, storage instructions, and/or instructions for manipulation of products requiring mixing or reconstitution. The kit may also include instructions for detection read out and interpretation.

By “level” is meant a level of a protein, glycan, or mRNA, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein, glycan, or mRNA in a sample. By “protein level profile” is meant one or more protein level values determined for a sample.

By “processing a sample” is meant any process carried out on the sample prior to the determination of the level or expression of the protein. Exemplary processing steps include, but are not limited to, centrifugation of the sample, fractionation of the sample, treatment with reagents (e.g., digesting enzymes or reducing reagents), and/or dilution of the sample. By “fractionation of a sample” is meant the general processes of separating the various components of a sample. For example, the components of the sample may be separated by chromatography (e.g., ion exchange chromatography). In some cases, the most abundant proteins, such as, proteins present at greater than 0.01, greater than 0.02, greater than 0.05, greater than 0.1 g/dL (e.g., greater than 0.2 g/dL, greater than 0.5 g/dL, greater than 1.0 g/dL, greater than 2.0 g/dL, greater than 3.0 g/dL) are depleted from the sample by chromatography to enhance the sensitivity for less abundant proteins, such as, proteins present at less than 0.2 g/dL (e.g., less than 0.1 g/dL, less than 0.05 g/dL, less than 0.01 g/dL). Columns/kits for the depletion of abundant proteins are known in the art, for example, MARS Human-6 and Human-7 from Agilent Technologies deplete the 6 and 7 most abundant proteins from human plasma.

By “reagent” is meant a polynucleotide sequence or polypeptide sequence capable of detecting a target sequence, or a fragment thereof.

By a “reference” is meant any useful reference used to compare protein or mRNA levels related to Kawasaki disease and/or binding of IgG to a peptide of Table 3. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having Kawasaki disease; a sample from a subject that is diagnosed with cardiac artery aneurysms or stenosis; a sample from a subject that has been treated for Kawasaki disease; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having Kawasaki disease; a subject that is diagnosed with cardiac artery aneurysms or stenosis; a subject that has been treated for Kawasaki disease. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

“Response” as used herein indicates a subject's response to a Kawasaki disease therapy, e.g., a response can be a positive response such that symptoms will be alleviated as a result of the Kawasaki disease therapy.

By “selecting a subject” is meant to choose a subject directly or indirectly in preference to others based on an analysis, e.g., analysis of results of the methods of the invention or clinical evaluation. Directly selecting means performing a process (e.g., performing an analysis) to choose a subject. Indirectly selecting refers to receiving the results of an analysis from another party or source (e.g., a third party laboratory that directly performed the analysis).

By “solid support” is meant a structure capable of storing, binding, or attaching one or more binding agents.

By “subject” is meant a human (e.g., a child less than 18 years old, less than 13 years old, less than 8 years old, less than 5 years old, less than 4 years old, less than 3 years old, less than 2 years old, or less than 1 year old). A subject to be treated with a pharmaceutical composition described herein may be one who has been diagnosed by a medical practitioner as having such a disease or condition (e.g., Kawasaki disease) or one at risk for developing a disease or condition (e.g., cardiac artery aneurysm or stenosis).

By “target sequence” is meant a portion of a gene or a gene product, including the mRNA and related cDNA.

By “therapeutic agent” is meant any agent that produces a healing, curative, stabilizing, or ameliorative effect.

A “therapeutically effective amount” of a compound may vary according to factors such as the disease state, age, sex, and weight of the individual, and the ability of the compound to elicit a desired response in the individual. A therapeutically effective amount encompasses an amount in which any toxic or detrimental effects of the compound are outweighed by the therapeutically beneficial effects. A therapeutically effective amount also encompasses an amount sufficient to confer benefit, e.g., clinical benefit.

By “treating” is meant administering a composition (e.g., a pharmaceutical composition) for therapeutic purposes or administering treatment to a subject already having a condition or disorder to improve the subject's condition or to reduce the likelihood of a condition or disorder. By “treating a condition or disorder” is meant that the condition or disorder and/or the symptoms associated with the condition or disorder are, e.g., alleviated, reduced, cured, or placed in a state of remission. By “reduce the likelihood of” is meant reducing the severity, the frequency, and/or the duration of a disorder (e.g., cardiac artery aneurysms and/or stenosis) or symptoms thereof. Reducing the likelihood of cardiac artery aneurysms and/or stenosis is synonymous with prophylaxis or the chronic treatment of cardiac artery aneurysms and/or stenosis.

Other features and advantages of the invention will be apparent from the following Detailed Description and the claims.

### DETAILED DESCRIPTION OF THE INVENTION

There is no specific test available to diagnose Kawasaki disease. Diagnosis largely is a process of ruling out diseases that cause similar signs and symptoms, including: scarlet fever; juvenile rheumatoid arthritis; Stevens-Johnson syndrome; toxic shock syndrome; measles; certain tick-borne illnesses. A doctor may do a physical examination and perform other tests to help in the diagnosis or prognosis. These tests may include urine tests, blood tests, electrocardiogram, and echocardiogram. (http://www.mayoclinic.org/diseases-conditions/kawasaki-disease/basics/tests-diagnosis/con-20024663, Mar. 3, 2014).

The present invention relates to the identification of biomarkers (e.g., protein levels, mRNA levels, glycan abundance, or IgG binding) that identify subjects having Kawasaki disease and/or being predisposed to develop Kawasaki disease-related cardiac artery aneurysms or stenosis. Such differential levels of proteins, mRNAs, glycans, and/or differential binding of IgG in samples can be used to diagnose, prognose, and classify subjects with Kawasaki disease and/or a predisposition to develop cardiac artery aneurysms or stenosis from healthy controls. Accordingly, the kits and methods described herein are useful for treating or diagnosing Kawasaki disease and/or related cardiac artery aneurysms or stenosis. Also described herein are diagnostic kits (e.g., on a solid support, such as an array or chip) which can be used to perform such methods.

### Proteins

Applicants have discovered that the levels of certain proteins can be utilized to diagnose, prognose, and treat Kawasaki disease, as well as to select subjects who would benefit from either IVIG therapy or a Kawasaki disease therapy other than, or in addition to, IVIG therapy. Proteins, the levels of which are of interest in the methods and compositions of the invention, include those in Table 18.

TABLE 18
Proteins with levels relevant to Kawasaki disease

| | Accession | Gene |
|---|---|---|
| Protein Name | No. | Symbol |
| collagen, type VI, alpha 3 | E7ENL6 | COL6A3 |
| F-box protein 47 | Q5MNV8 | FBXO47 |
| collagen, type VI, alpha 1 | P12109 | COL6A1 |
| androgen receptor | P10275 | AR |
| tumor protein p53 binding protein 1 | Q12888 | TP53BP1 |
| peptidase inhibitor 16 | Q6UXB8 | PI16 |
| cadherin 13 | P55290 | CDH13 |
| tenascin XB | G5E9A9 | TNXB |
| tenascin XB | B0UYX3 | TNXB |
| peroxisome proliferator-activated | Q07869 | PPARA |
| receptor alpha | | |
| collagen, type I, alpha 1 | P02452 | COL1A1 |
| Ig alpha-2 chain C region (A2m | P01877 | IGHA2 |
| marker) | | |
| Ig heavy chain V-III region TIL | P01765 | HV304 |
| Ig alpha-1 chain C region | P01876 | IGHA1 |
| olfactomedin 1 | Q6IMJ5 | OLFM1 |
| complement factor H | Q5TFM2 | CFH |
| prostaglandin D2 synthase 21 kDa | P41222 | PTGDS |
| (brain) | | |
| sex hormone-binding globulin | I3L145 | SHBG |
| Ig kappa chain V-I region | P01593 | KV101 |
| gelsolin | P06396 | GSN |
| Ig lambda-1 chain C regions (Mcg | P0CG04 | IGLC1 |
| marker) | | |
| BMP2 inducible kinase | Q9NSY1 | BMP2K |
| serpin peptidase inhibitor, clade C | P01008 | SERPINC1 |
| (antithrombin), member 1 | | |
| pregnancy-zone protein | P20742 | PZP |
| inter-alpha-trypsin inhibitor heavy | P19827 | ITIH1 |
| chain 1 | | |
| Ig kappa chain V-II region | P01617 | KV204 |
| inter-alpha-trypsin inhibitor heavy | P19823 | ITIH2 |
| chain 2 | | |
| complement factor H | P08603 | CFH |
| Hepatocyte growth factor activator | Q04756 | HGFAC |
| attractin | O75882 | ATRN |
| kininogen 1 | P01042 | KNG1 |
| coagulation factor XIII, B | P05160 | F13B |
| polypeptide | | |
| complement factor B | B4E1Z4 | CFB |
| complement component 1, r | H0YFH3 | C1R |
| subcomponent | | |
| complement component 1, r | P00736 | C1R |
| subcomponent | | |
| extracellular matrix protein 1 | Q16610 | ECM1 |
| complement component 1, s | P09871 | C1S |
| subcomponent | | |
| kininogen 1 | C9JEX1 | KNG1 |
| complement component 6 | P13671 | C6 |
| vitamin D binding protein (group- | P02774 | GC |
| specific component) | | |
| Alpha-1-antichymotrypsin(serpin | P01011 | SERPINA3 |
| peptidase inhibitor, clade A, | | |
| member 3) | | |
| lactate dehydrogenase C | F5H5G7 | LDHC |
| Alpha-1-antichymotrypsin(serpin | G3V3A0 | SERPINA3 |
| peptidase inhibitor, clade A, | | |
| member 3) | | |
| armadillo repeat containing 2 | Q8NEN0 | ARMC2 |
| unc-45 homolog A (C. elegans) | Q9H3U1 | UNC45A |
| defensin, alpha 1 | P59665 | DEFA1 |
| S100 calcium binding protein A9 | P06702 | S100A9 |
| solute carrier family 26 (anion | P40879 | SLC26A3 |
| exchanger), member 3 | | |
| zeta-chain (TCR) associated protein | P43403 | ZAP70 |
| kinase 70 kDa | | |
| Zinc finger protein 106 homolog | Q9H2Y7 | ZFP106 |
| apolipoprotein B | P04114 | APOB |
| Zinc finger protein 161 homolog | J3QLI2 | ZFP161 |
| apolipoprotein M | O95445 | APOM |
| apolipoprotein C-II | P02655 | APOC2 |
| apolipoprotein F | F5GXS5 | APOF |
| zinc finger protein 578 | I3L1Y6 | ZNF578 |
| xin actin-binding repeat containing 2 | A4UGR9 | XIRP2 |
| lipoprotein, Lp(a) | P08519 | LPA |
| armadillo repeat containing 10 | Q8N2F6 | ARMC10 |
| olfactory receptor, family 5, | Q8NGL3 | OR5D14 |
| subfamily D, member 14 | | |
| orosomucoid 1 | P02763 | ORM1 |
| Alpha-1-antitrypsin (serpin | P01009 | SERPINA1 |
| peptidase inhibitor, clade A, | | |
| member 1) | | |
| nitric oxide synthase 2, inducible | 4843 | NOS2 |
| (Gene) | | |
| carcinoembryonic antigen-related | 1088 | CEACAM8 |
| cell adhesion molecule 8 (Gene) | | |
| interleukin 33 (Gene) | 90865 | IL33 |
| interleukin 6 (interferon, beta 2) | 3569 | IL6 |
| (Gene) | | |
| Plasminogen | P00747 | PLG |
| Vitamin K-dependent protein C | P04070 | PROC |
| Coagulation factor XI | P03951 | F11 |
| Apolipoprotein F | F5GXS5 | APOF |
| CD44 antigen | E7EPC6 | CD44 |
| Ankyrin repeat domain-containing | Q9UPS8 | ANKRD26 |
| protein 26 | | |
| Lysosome-associated membrane | B4E2S7 | LAMP2 |
| glycoprotein 2 | | |
| Basal cell adhesion molecule | P50895 | BCAM |
| Multimerin-1 | Q13201 | MMRN1 |
| Transforming growth factor-beta- | G8JLA8 | TGFBI |
| induced protein ig-h3 | | |
| Methylcytosine dioxygenase TET2 | E7EQS8 | TET2 |
| Alpha-1-antichymotrypsin | P01011 | SERPINA3 |
| Cystatin-C | P01034 | CST3 |
| matrix metallopeptidase 1 | 4312 | MMP1 |
| (interstitial collagenase) (Gene) | | |
| Aftiphilin | Q6uLP2 | AFTPH |
| Death domain containing 1 | Q6ZMT9 | DTHD1 |
| Metastasis associated 1 family, | E7EV10 | MTA |
| member 3 | | |
| HGF activator | Q04756 | HGFAC |
| Bone marrow stromal cell antigen 1 | Q10588 | BST1 |
| Mannan-binding lectin serine | O00187 | MASP2 |
| peptidase 2 | | |
| HCG2014417, isoform CRA a | B7Z718 | AGAP2 |
| T-lymphoma invasion and | F5GZ53 | TIAM1 |
| metastasis-inducing protein 1 | | |
| Hemoglobin subunit gamma-1 | P69891 | HBG1 |
| Hemoglobin subunit gamma-2 | P69892 | HBG2 |
| Protein S100-A9 | P06702 | S100A9 |
| Protein S100-A8 | P05109 | S100A8 |
| Leucine-rich alpha-2-glycoprotein | P02750 | LRG1 |
| Actin, cytoplasmic 1 | P60709 | ACTB |
| Haptoglobin-related protein | P00739 | HPR |
| Cathepsin D | P07339 | CTSD |
| C-reactive protein | P02741 | CRP |
| Kallistatin | P29622 | SERPINA4 |
| Kininogen-1 | C9JEX1 | KNG1 |
| Lumican | P51884 | LUM |
| Alpha-2-HS-glycoprotein | P02765 | AHSG |
| Butyrylcholinesterase | P06276 | BCHE |
| Coagulation factor IX | P00740 | F9 |
| Ig heavy chain V-III region BRO | P01766 | HV305 |
| Selenoprotein P | P49908 | SEPP1 |
| Cadherin-13 | P55290 | CDH13 |
| Myosin-8 | P13535 | MYH8 |
| Orosomucoid 2 | P19652 | ORM2 |
| Polycystin (PKD) family receptor for | Q9NTG1 | PKDREJ |
| egg jelly | | |
| Complement component 9 | P02748 | C9 |
| Complement component 2 | P06681 | C2 |
| Complement factor H-related 3 | Q6NSD3 | CFHR3 |
| Clusterin | P10909 | CLU |
| Kallikrein B, plasma (Fletcher | H0YAC1 | KLKB1 |
| factor) 1 | | |
| Macrophage stimulating 1 | P26927 | MST1 |
| (hepatocyte growth factor-like) | | |
| Biotinidase | F8W1Q3 | BTD |
| Peptidoglycan recognition protein 2 | Q96PD5 | PGLYRP2 |
| Fibulin 1 | B1AHL2 | FBLN1 |
| fibulin 1 | P23142 | FBLN1 |
| Mannan-binding lectin serine | P48740 | MASP1 |
| peptidase 1 (C4/C2 activating | | |
| component of Ra-reactive factor) | | |
| Alpha-1-microglobulin/bikunin | P02760 | AMBP |
| precursor | | |
| Mannan-binding lectin serine | F8W876 | MASP1 |
| peptidase 1 (C4/C2 activating | | |
| component of Ra-reactive factor) | | |
| Apolipoprotein H (beta-2- | P02749 | APOH |
| glycoprotein I) | | |
| TBC1 domain family, member 8B | J3KN75 | TBC1D8B |
| (with GRAM domain) | | |
| Afamin | P43652 | AFM |
| Heat shock 70 kDa protein 5 | P11021 | HSPA5 |
| (glucose-regulated protein, 78 kDa) | | |
| IgA | NA | IGA |
| ST6 beta-galactosamide alpha-2,6- | NA | ST6 |
| sialyltranferase 1 | | |
| haptoglobin | P00738 | HP |
| Matrix metallopeptidase 9 | P14780 | MMP9 |
| Nidogen-1 | P14543 | NID1 |
| Neural cell adhesion molecule L1- | O00533 | CHL1 |
| like protein | | |
| Coagulation factor X | P00742 | F10 |
| Titin | Q8WZ42 | TTN |
| Insulin-like growth factor binding | P35858 | IGFALS |
| protein, acid labile subunit | | |
| Glycosylphosphatidylinositol | P80108 | GPLD1 |
| specific phospholipase D1 | | |
| Retinol binding protein 4, plasma | Q5VY30 | RBP4 |
| Centrosomal protein 70 kDa | Q8NHQ1 | CEP70 |
| Thrombospondin 4 | P35433 | THBS4 |
| Fibronectin 1 | F8W7G7 | FN1 |
| Insulin-like growth factor 2 | P01344 | IGF2 |
| (somatomedin A) | | |
| Neuropilin 1 | O14786 | NRP1 |
| Neural cell adhesion molecule 1 | E9PLH7 | NCAM1 |
| alpha-1-B glycoprotein | P04217 | A1BG |
| Coagulation factor XIII, A1 | P00488 | F13A1 |
| polypeptide | | |
| Apolipoprotein C-1 | P02654 | APOC1 |
| Carboxypeptidase | Q96IY4 | CPB2 |
| Zinc finger protein 217 | O75362 | ZNF217 |
| Collagen, type XI, alpha 2 | H0YHY3 | COL11A2 |
| Selectin L | P14151 | SELL |
| Ectonucleotide pyrophosphatase/ | Q13822 | ENPP2 |
| phosphodiesterase 2 | | |
| Coagulation factor II (thrombin) | P00734 | F2 |
| Apolipoprotein D | P05090 | APOD |
| Insulin growth factor | P17936 | IGFBP3 |
| binding protein 3 | | |
| Dehydrogenase/reductase (SDR | A8MXC2 | DHRS11 |
| family) member 11 | | |
| Quiescin Q6 sulfhydryl oxidase 1 | O00391 | QSOX1 |
| Lipopolysaccharide binding protein | P18428 | LBP |
| Hemaglobin, alpha 1 | P69905 | HBA1 |
| Hemaglobin, delta | P02042 | HBD |
| Fc fragment of IgG, low affinity IIIa | P08637 | FCGR3A |
| receptor (CD16a) | | |
| Hemaglobin, gamma G | P69892 | HBG2 |
| Hemaglobin, gamma A | P69891 | HBG1 |
| Hemaglobin, beta | P68871 | HBB |
| Kinesin family member 20B | Q96Q89 | KIF20B |
| Lysozyme | P61626 | LYZ |
| Actin, alpha, cardiac muscle 1 | P68032 | ACTC1 |
| Inter-alpha-trypsin inhibitor heavy | Q06033 | ITIH3 |
| chain 3 | | |
| Inter-alpha-trypsin inhibitor heavy | Q14624 | ITIH4 |
| chain family, member 4 | | |
| Inter-alpha-trypsin inhibitor heavy | B7ZKJ8 | ITIH4 |
| chain family, member 4 | | |
| Inter-alpha-trypsin inhibitor heavy | E9PGN5 | ITIH4 |
| chain family, member 4 | | |
| Actin, beta-like 2 | Q562R\grave{ } | ACTBL2 |
| Hemaglobin, epsilon 1 | P02100 | HBE1 |
| Protein phosphatase 6, regulatory | B3KMJ7 | PPP6R2 |
| subunit 2 | | |
| Dedicator of cytokinesis 3 | Q8IZD9 | DOCK3 |
| Centrosomal protein 290 kDa | O15078 | CEP290 |

As indicated above, proteins useful for diagnosing Kawasaki disease, or selecting or classifying a subject that may benefit from a Kawasaki disease therapy include those in Tables 1 and 2. Proteins useful for selecting or classifying a subject that may benefit from a therapy other than, or in addition to, IVIG therapy include those in Tables 4, 5, 6, and 7.

Applicants have discovered that the level of binding of IgG in samples to certain proteins can be utilized to diagnose, prognose, and treat Kawasaki disease, as well as to select subjects who would benefit from a Kawasaki disease therapy. Proteins, the binding of which are of interest in the methods and compositions of the invention, include those comprising an amino acid sequence of any one of SEQ ID NOs:1 to 68.

### Genes

Applicants have discovered that the mRNA expression levels of certain genes can be utilized to diagnose, prognose, and treat Kawasaki disease, as well as to select subjects who would benefit from either IVIG therapy or a Kawasaki disease therapy other than, or in addition to, IVIG therapy. Genes, the mRNA levels of which are of interest in the methods and compositions of the invention, include those in Table 19.

TABLE 19
Genes with mRNA levels relevant to Kawasaki disease

| | Accession | Gene |
|---|---|---|
| Gene Name | No. | Symbol |
| CD80 molecule | 941 | CD80 |
| Matrix metallopeptidase 9 | 4318 | MMP9 |
| (gelatinase B, 92 kDa gelatinase, | | |
| 92 kDa type IV collagenase) | | |
| Colony stimulating factor 2 | 1439 | CSF2RB |
| receptor, beta, low-affinity | | |
| Tumor necrosis factor receptor | 7132 | TNFRSF1A |
| superfamily, member 1A | | |
| carcinoembryonic antigen- | 1088 | CEACAM8 |
| related cell adhesion molecule 8 | | |
| Beta-glucuronidase | 2990 | GUSB |
| S100 calcium binding protein A12 | 6283 | S100A12 |
| Versican | 1462 | VCAN |
| Fc fragment of IgG, high affinity | 2209 | FCGR1A |
| Ia, receptor (CD64) | | |
| S100 calcium binding protein A11 | 6282 | S100A11 |
| Tumor necrosis factor receptor | 7133 | TNFRSF1B |
| superfamily, member 1B | | |
| Interleukin 1, beta | 3553 | IL1B |
| Interleukin 1 receptor antagonist | 3557 | IL1RN |
| S100 calcium binding protein A8 | 6279 | S100A8 |
| Tumor necrosis factor (ligand) | 10673 | TNFSF13B |
| superfamily, member 13b | | |
| Killer cell lectin-like receptor | 10219 | KLRG1 |
| subfamily G, member 1 | | |
| Fc fragment of IgG, high affinity | 2212 | FCGR2A |
| IIa, receptor (CD32) | | |
| chemokine (C-C motif) receptor 2 | 729230 | CCR2 |
| Actin, beta | 60 | ACTB |
| Tumor necrosis factor | 7124 | TNF |

As indicated above, genes useful for selecting or classifying a subject that may benefit from a therapy other than, or in addition to, IVIG therapy include those in Table 10, Table 11, Table 12, and/or Table 13.

### Glycans

Applicants have discovered that the levels of certain glycans on IgG and IgA can be utilized to diagnose, prognose, and treat Kawasaki disease, as well as to select subjects who would benefit from either IVIG therapy or a Kawasaki disease therapy other than, or in addition to, IVIG therapy. Glycans, the levels of which are of interest in the methods and compositions of the invention, include those in Table 20.

TABLE 20
Glycans with levels relevant to Kawasaki disease

| Fraction | Structure |
|---|---|
| GP6HA | FA2[3]G1 |
| GP4HA | A2[6]BG1 |
| GP26HA | FA2BG2S[3,6]2 |
| | FA2BG2S[6,6]2 |
| GP11HA | FA2G2 |
| GP5HA | FA2[6]G1 |
| GP7HA | FA2[6]BG1 |
| GP2HA | FA2 |
| GP12HA | FA2BG2 |
| IgG2_G0F_BGlcNAc | |
| IgG2_BGlcNAc | |
| GP3HA | M5 |
| | FA2B |
| | A2[6]G1 |
| GP38HA | A4G4S[3,3,3,3]4 |
| GP23HA | A2G2S[3,6]2 |
| IgG2_G1F_BGlcNAc | |
| GP13HA | A2[3]BG1S[3]1 |
| | A2[3]BG1S[6]1 |
| | M7 D1 |
| | FA2[3]G1S[3]1 |
| | FA2[3]G1S[6]1 |
| GP10HA | A2BG2 |
| GP41HA | A4G4S[3,3,3,3]4 |
| GP28HA | A3BG3S[3,6]2 |
| IgG3_4_A1 | IgG3 isoform |
| | A2F1G1S1 |
| GP37HA | A4F1G3S[3,3,6]3 |
| | A4F1G3S[3,6,6]3 |
| GP42HA | A4F1G4S[3,3,3,6]4 |
| GP43HA | A4G4LacS[3,3,3,6]4 |
| GP43LA | A4G4LacS[3,3,3,6]4 |
| GP44HA | A4F3G4S[3,3,3,3]4 |
| GP44LA | A4F3G4S[3,3,3,3]4 |
| GP9HA | A1[3]G1S[3]1 |
| | A2G2 |
| GP9LA | A1[3]G1S[3]1 |
| | A2G2 |
| GP12LA | FA2BG2 |
| | A2[3]BG1S[3]1 |
| | A2[3]BG1S[6]1 |
| GP14HA | FA2[3]BG1S[3]1 |
| GP14LA | FA2[3]BG1S[3]1 |
| GP16LA | A2BG2S[6]1 |
| GP18HA | FA2G2S[6]1 |
| | FA2BG2S[3]1 |
| GP18LA | FA2G2S[6]1 |
| | FA2BG2S[3]1 |
| GP19LA | FA2BG2S[6]1 |
| GP20HA | A2G2S[3,6]2 |
| | A3G3S[3]1 |
| GP20LA | A2G2S[3,6]2 |
| | A3G3S[3]1 |
| GP24LA | A2BG2S[6,6]2 |
| GP29LA | A4G4S[3]1 |
| GP32HA | A3G3S[3,3,6]3 |
| GP32LA | A3G3S[3,3,6]3 |
| GP34HA | A3G3S[3,3,6]3 |
| GP34LA | A3G3S[3,3,6]3 |
| GP38LA | A4G4S[3,3,3,3]4 |
| GP3LA | M5 |
| | FA2B |
| | A2[6]G1 |
| GP41LA | A4G4S[3,3,3,3]4 |
| GP42LA | A4F1G4S[3,3,3,6]4 |
| GP42HA | A4F1G4S[3,3,3,6]4 |
| GP41HA | A4G4S[3,3,3,3]4 |
| GP43LA | A4G4LacS[3,3,3,6]4 |
| GP43HA | A4G4LacS[3,3,3,6]4 |
| GP37HA | A4F1G3S[3,3,3]3 |
| | A4F1G3S[3,3,6]3 |
| | A4F1G3S[3,6,6]3 |
| GP44HA | A4F3G4S[3,3,3,3]4 |
| GP44LA | A4F3G4S[3,3,3,3]4 |
| GP23HA | A2G2S[3,6]2 |
| GP35HA | A3F1G3S[3,3,3]3 |
| GP31HA | A3G3S[3,3,3]3 |
| IgA_G1_S1 | A2,G1,S1 |
| GP13HA | A2[3]BG1S[3]1 |
| | A2[3]BG1S[6]1 |
| | M7 D1 |
| GP18HA | FA2G2S[6]1 |
| GP12LA | FA2BG2 |
| GP20LA | A2G2S[3,6]2 |
| GP18LA | FA2G2S[6]1 |
| GP12HA | FA2BG2 |
| GP11HA | FA2G2 |
| GP19HA | FA2BG2S[3]1; |
| | FA2BG2S[6]1 |
| GP3HA | M5; FA2B; A2[6]G1 |
| GP10HA | A2BG2 |
| GP5HA | FA2[6]G1 |
| GP6HA | FA2[3]G1 |
| GP13LA | A2[3]BG1S[3]1; |
| | A2[3]BG1S[6]1; |
| | M7 D1; |
| | FA2[3]G1S[3]1 |
| GP24LA | A2BG2S[6,6]2 |
| GP8HA | FA2[3]BG1; M6 D3 |
| GP7HA | FA2[6]BG1 |
| GP32LA | A3G3S[3,3,6]3 |
| GP26HA | FA2BG2S[3,6]2 |
| | FA2BG2S[6,6]2 |
| GP5LA | FA2[6]G1 |
| GP19LA | FA2BG2S[3]1 |
| | FA2BG2S[6]1 |
| GP6LA | FA2[3]G1 |
| GP2HA | FA2 |
| GP20HA | A2G2S[3,6]2 |

G; N-glycans, P; plasma, fraction #, LA; low abundant fraction, HA; high abundant fraction.

Glycan annotations according to Oxford Symbol nomenclature. All N-glycans have two core GlcNAcs; F at the start of the abbreviation indicates a core a(1-6)fucose linked to inner GlcNAc; Mx, number (x) of mannose on core GlcNAcs; Ax, number of antenna (GlcNAc) on trimannosyl core; A2, biantennary with both GlcNAcs as b(1-2) linked; A3, triantennary with a GlcNAc linked b(1-2) to both mannose and a third GlcNAc linked b(1,4) to the a(1-3) linked mannose; A3′; triantennary with a GlcNAc linked b(1-2) to both mannose and the third GlcNAc linked b(1-6) mannose; B, bisecting GlcNAc linked b(1-4) to b(1-3) mannose; Gx, number (x) of b1-4 linked galasose on the antenna; Fx, number (x) of linked fucose on antenna, (4) or (3) after the F indicates that the Fuc is a(1-4) or a(1-3) linked to the GlcNAc; Sx, number (x) sialic acids linked to galactose; the number 3 or 6 in parenthesis after S indicates whether the sialic acid is in an a(2-3) or a(2-6) linkage. See Harvey et al. Proposal for a standard system for drawing structural diagrams of N- and O-linked carbohydrates and related compounds. Proteomics 2009, 9:3796-801.

As indicated above, glycans useful for diagnosing Kawasaki disease, selecting or classifying a subject that may benefit from a Kawasaki disease therapy and/or selecting or classifying a subject that may benefit from a therapy other than, or in addition to, IVIG therapy include those in Table 14, Table 15, Table 16, and/or Table 17.

### Diagnostic and Classification Methods

The present invention features methods and compositions to diagnose Kawasaki disease. The kits and methods of the invention may be used alone or as a companion diagnostics with other diagnostic or therapeutic approaches, as an early molecular screen to distinguish Kawasaki disease from other diseases and disorders with similar symptoms. More specifically, alterations in the level of one or more proteins described herein (e.g., proteins of Table 1 and/or Table 2) and/or binding of IgG to a protein of Table 3 in a test sample as compared to a normal reference can be used to diagnose Kawasaki disease and/or distinguish Kawasaki disease from diseases or disorders with similar symptoms, thereby allowing subject classification.

Further, the present invention features methods and compositions useful in determining the likelihood of a subject to develop cardiac artery aneurysms and/or stenosis. For example, the methods and compositions of the invention may be used to determine if a subject may benefit from a Kawasaki disease therapy other than, or in addition to, IVIG therapy, by determining the levels of one or more biomarkers of Table 4, Table 5, Table 6, Table 7, Table 8, Table 9, Table 10, Table 11, Table 12, Table 13, Table 14, Table 15, Table 16, and/or Table 17.

The methods of the invention can be used to diagnose, prognose, or classify a subject, for example, an increase in the level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more, as compared to a reference) of the biomarker(s) (e.g., a protein of Table 1) may indicate a subject has Kawasaki disease and/or may benefit from a Kawasaki disease therapy. Similarly, a decrease in the level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less, as compared to a reference) of the biomarker(s) (e.g., a protein of Table 2) may indicate a subject has Kawasaki disease and/or may benefit from a Kawasaki disease therapy. An increase (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more, as compared to a reference) in binding of IgG in a sample to a protein comprising an amino acid sequence of Table 3 may indicate a subject has Kawasaki disease and/or may benefit from a Kawasaki disease therapy.

Alternatively, an increase in the level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more, as compared to a reference) of the biomarker(s) (e.g., a protein of Table 4) may indicate a subject is predisposed to develop cardiac artery aneurysms or stenosis and/or may benefit from a Kawasaki disease therapy other than, or in addition to, IVIG therapy. Similarly, a decrease in the level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less, as compared to a reference) of a biomarker(s) (e.g., a protein of Table 5) may indicate a subject is predisposed to develop cardiac artery aneurysms or stenosis and/or may benefit from a Kawasaki disease therapy other than, or in addition to, IVIG therapy.

To carry out the methods of the invention, a sample can be obtained by any method known in the art. For instance, samples from a subject may be obtained by venipuncture, resection, bronchoscopy, fine needle aspiration, bronchial brushings, or from sputum, pleural fluid, urine, or blood, such as serum or plasma. Proteins can be detected in these samples. By screening such biological samples, a simple early diagnosis or differential diagnosis can be achieved for Kawasaki disease. In addition, the progress of therapy can be monitored by testing such biological samples for target proteins and/or binding of IgG to a protein of Table 3. Furthermore, the prediction of outcome or response to therapy can similarly be tested using such biological samples for target proteins and/or binding of IgG to a protein of Table 3.

In certain embodiments, the sample may be contacted with an antibody specific for the target protein under conditions sufficient for an antibody-protein complex to form, and detection of the complex. The presence of the biomarker may be detected in a number of ways, such as by Western blotting or ELISA procedures using any of a wide variety of tissues or samples, including plasma or serum. A wide range of immunoassay techniques using such an assay format are available, see, e.g., U.S. Pat. Nos. 4,016,043, 4,424,279, and 4,018,653. These include both single-site and two-site or “sandwich” assays of the noncompetitive types, as well as traditional competitive binding assays. These assays also include direct binding of a labeled antibody to a target biomarker.

Another method involves immobilizing the target biomarkers (e.g., on a solid support) and then exposing the immobilized target to a specific antibody, which may or may not contain a label. Depending on the amount of target and the strength of the label's signal, a bound target may be detectable by direct labeling with the antibody. Alternatively, a second labeled antibody, specific to the first antibody is exposed to the target-first antibody complex to form a target-first antibody-second antibody tertiary complex. The complex is detected by the signal emitted by a label, e.g., an enzyme, a fluorescent label, a chromogenic label, a radionuclide containing molecule (i.e., a radioisotope), or a chemiluminescent molecule.

Variations on the forward assay include a simultaneous assay, in which both sample and labeled antibody are added simultaneously to a bound antibody. These techniques are well known to those skilled in the art, including any minor variations as will be readily apparent. In a typical forward sandwich assay, a first antibody having specificity for the biomarker is either covalently or passively bound to a solid surface (e.g., a glass or a polymer surface, such as those with solid supports in the form of tubes, beads, discs, or microplates), and a second antibody is linked to a label that is used to indicate the binding of the second antibody to the molecular marker.

In alternative methods, the expression of a protein in a sample may be examined using immunohistochemistry (“IHC”) and staining protocols. IHC staining of tissue sections has been shown to be a reliable method of assessing or detecting presence of proteins in a sample. IHC and immunofluorescence techniques use an antibody to probe and visualize cellular antigens in situ, generally by chromogenic or fluorescent methods. The tissue sample may be fixed (i.e., preserved) by conventional methodology (see, e.g., “Manual of Histological Staining Method of the Armed Forces Institute of Pathology,” 3^{rd} edition (1960) Lee G. Luna, HT (ASCP) Editor, The Blakston Division McGraw-Hill Book Company, New York; The Armed Forces Institute of Pathology Advanced Laboratory Methods in Histology and Pathology (1994) Ulreka V. Mikel, Editor, Armed Forces Institute of Pathology, American Registry of Pathology, Washington, D.C.). One of skill in the art will appreciate that the choice of a fixative is determined by the purpose for which the sample is to be histologically stained or otherwise analyzed. By way of example, neutral buffered formalin, Bouin's or formaldehyde, may be used to fix a sample. Generally, the sample is first fixed and is then dehydrated through an ascending series of alcohols, infiltrated and embedded with paraffin or other sectioning media so that the tissue sample may be sectioned. Alternatively, one may section the tissue and fix the sections obtained. The primary and/or secondary antibody used for immunohistochemistry typically will be labeled with a detectable moiety, such as a radioisotope, a colloidal gold particle, a fluorescent label, a chromogenic label, or an enzyme-substrate label.

Alternatively, the levels of biomarkers may be detected without the use of binding agents. In some instances, biological samples as described herein are analyzed, for example, by one or more, enzymatic methods, chromatographic methods, mass spectrometry (MS) methods, chromatographic methods followed by MS, electrophoretic methods, electrophoretic methods followed by MS, nuclear magnetic resonance (NMR) methods, and combinations thereof. In some instances, the biological sample is treated with one or more enzymes (e.g., trypsin). Exemplary chromatographic methods include, but are not limited to, Strong Anion Exchange chromatography using Pulsed Amperometric Detection (SAX-PAD), liquid chromatography (LC), high performance liquid chromatography (HPLC), ultra performance liquid chromatography (UPLC), thin layer chromatography (TLC), amide column chromatography, and combinations thereof. Exemplary mass spectrometry (MS) include, but are not limited to, tandem MS, LC-MS, LC-MS/MS, matrix assisted laser desorption ionisation mass spectrometry (MALDI-MS), Fourier transform mass spectrometry (FTMS), ion mobility separation with mass spectrometry (IMS-MS), electron transfer dissociation (ETD-MS), Multiple Reaction Monitoring (MRM), and combinations thereof. Exemplary electrophoretic methods include, but are not limited to, capillary electrophoresis (CE), CE-MS, gel electrophoresis, agarose gel electrophoresis, acrylamide gel electrophoresis, SDS-polyacrylamide gel electrophoresis (SDS-PAGE) followed by Western blotting using antibodies that recognize specific glycan structures, and combinations thereof. Exemplary nuclear magnetic resonance (NMR) include, but are not limited to, one-dimensional NMR (1D-NMR), two-dimensional NMR (2D-NMR), correlation spectroscopy magnetic-angle spinning NMR (COSY-NMR), total correlated spectroscopy NMR (TOCSY-NMR), heteronuclear single-quantum coherence NMR (HSQC-NMR), heteronuclear multiple quantum coherence (HMQC-NMR), rotational nuclear overhauser effect spectroscopy NMR (ROESY-NMR), nuclear overhauser effect spectroscopy (NOESY-NMR), and combinations thereof.

Any of the methods herein that rely upon protein measurement can also be adapted for use with the measurement of mRNA levels for the protein. The level of mRNA can be determined using methods known in the art. Methods to measure mRNA levels generally include, but are not limited to, sequencing, northern blotting, RT-PCR, gene array technology, and RNAse protection assays.

Binding Agents

Any binding agent that specifically binds a target biomarker may be used in the methods of the invention. The binding agent may be, e.g., a protein (e.g., an antibody), small molecule, or aptamer capable of specifically binding a target.

Preferably, each binding agent specifically binds to one biomarker in a sample. For determining the level of a biomarker, the measurement of antibodies specific to a biomarker of the invention in a subject may be used for the diagnosis of Kawasaki disease. The binding agent may optionally contain a label, such as a radioisotope, a colloidal gold particle, a fluorescent label, a chromogenic label, an enzyme-substrate label, or a chemiluminescent label.

Sample Processing

In some embodiments of any of the foregoing methods, the biological sample is processed prior to determining the level of the one or more the biomarkers, e.g., the biological sample is centrifuged, the biological sample is filtered, the biological sample is diluted, the biological sample is treated with reagents (e.g., digesting enzymes or reducing reagents), the biological sample is fractionated to remove more abundant proteins (e.g., proteins present at concentrations greater than 0.01 g/dL, greater than 0.02 g/dL, greater than 0.05 g/dL, greater than 0.1 g/dL, greater than 0.2 g/dL, greater than 0.5 g/dL, greater than 1.0 g/dL, greater than 2.0 g/dL, greater than 3.0 g/dL), such as, albumins, globulins (e.g., haptoglobulin, alpha2-macroglobulin, IgG, IgA, and IgM), alpha1-acid glycoprotein, apolipoprotein AI, apolipoprotein AII, complement C3, transthyretin, antitrypsin, transferrin, and fibrinogen and/or enrich for less abundant proteins, such as, any protein from Tables 1, 2, 4, and/or 5.

For example, a blood sample may be obtained, and prior to determining the level of one or more proteins, the sample may be centrifuged to remove red blood cells (i.e., to provide a plasma sample). The plasma sample may be spin filtered and diluted. Subsequently, the sample may be chromatographically separated using an immunoaffinity-based column to remove more abundant proteins (e.g., the 10-20, e.g., 10, 12, 14, 16, 18, 20 most abundant proteins) and enrich for less abundant proteins. The enriched sample may be concentrated and buffer exchanged, followed by treatment with a digesting enzyme, e.g., trypsin. Determination of protein levels may then be carried out on the processed sample.

### Methods for Predicting and Monitoring Response to Kawasaki Disease Therapies

The invention further features methods for predicting response to a Kawasaki disease therapy in a subject before or after administration of one or more Kawasaki disease therapies. These methods may be carried out generally as described above or as known in the art with respect to sample collection and assay format. For example, these methods may be carried out by collecting a sample, e.g., a blood or plasma sample from a subject; measuring the level of one or more biomarkers described herein (e.g., proteins of Table 1, 2, 4, 5, 6, 7, 8, and/or 9, genes of Table 10, Table 11, Table 12, and/or Table 13, and/or glycans of Table 14, Table 15, Table 16, and/or Table 17) in the sample and/or determining the binding of IgG in the sample to a protein comprising an amino acid sequence of Table 3; comparing to a control sample; and making a prediction about whether the subject will be responsive to a Kawasaki disease therapy. The method also can be used to predict whether a subject, who has been diagnosed with Kawasaki disease, will respond positively to a Kawasaki disease therapy such as a therapeutic (e.g., IVIG) or a combination of therapeutics (e.g., IVIG and one or more anticoagulants, an anti-inflammatory agent, and/or one or more immunosuppressant drugs).

A prediction of a positive response refers to a case where the Kawasaki disease symptoms will be alleviated and/or the risk of mortality will be reduced as a result of the Kawasaki disease therapy.

In the methods of predicting response to a Kawasaki disease therapy, the level of the protein(s) binding of IgG, gene(s), and/or glycan(s) can be determined relative to a control value. A control value can be a range or average value from a normal subject or a population of normal subjects; a value from a sample from a subject or population of subjects who have undergone a Kawasaki disease therapy and have reduced symptoms following therapy; a value from the same subject before the subject was diagnosed or before the subject started treatment.

The methods of the invention can be used to predict whether a subject will be responsive to a Kawasaki disease therapy, for example, an increase in the level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more, as compared to a reference) of the biomarker(s) (e.g., a protein of Table 1) may indicate a positive response to a Kawasaki disease therapy. Similarly, a decrease in the level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less, as compared to a reference) of the biomarker(s) (e.g., a protein of Table 2) may indicate a positive response to a Kawasaki disease therapy. Also, increased binding (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more, as compared to a reference) of IgG in the sample may indicate a positive response to a Kawasaki disease therapy.

Alternatively, an increase in the level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more, as compared to a reference) of the biomarker(s) (e.g., a protein of Table 4) may indicate a poor response to a Kawasaki disease therapy. Similarly, a decrease in the level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less, as compared to a reference) of the biomarker(s) (e.g., a protein of Table 5) may indicate a poor response to a Kawasaki disease therapy.

The methods of the invention can be used to predict a subject's response to a Kawasaki disease therapy and classify the subject as a “responder,” e.g., a subject with protein levels and/or binding of IgG indicative of a positive response to a Kawasaki disease therapy (e.g., IVIG), or a “non-responder,” e.g., a subject with protein levels and/or binding of IgG indicative of a poor response to a Kawasaki disease therapy (e.g., a subject that may benefit from a therapy other than, or in addition to, IVIG therapy).

The prediction can be made prior to administration of a first Kawasaki disease therapy. Alternatively, the prediction can be made after administration of the first Kawasaki disease therapy, or after administration of a first Kawasaki disease therapy but before a second Kawasaki disease therapy. Furthermore, the prediction can be made at any time during the course of a Kawasaki disease therapy.

The methods described herein can also be used to monitor Kawasaki disease status (e.g., progression or regression) during therapy or to optimize dosage of one or more therapeutic agents for a subject. For example, alterations (e.g., an increase or a decrease as compared to either the positive reference sample or the level diagnostic for Kawasaki disease) can be detected to indicate an improvement in Kawasaki disease status. In this embodiment, the levels of the protein(s), binding of IgG, gene(s), and/or glycan(s) may be measured repeatedly as a method of not only diagnosing disease, but also monitoring the treatment, prevention, or management of the disease.

In order to monitor the status of Kawasaki disease in a subject, subject samples may compared to reference samples taken early in the diagnosis of the disorder. Such monitoring may be useful, for example, in assessing the efficacy of a particular therapeutic agent (e.g., IVIG) in a subject, determining dosages, or in assessing disease progression or status. For example, levels of any of the proteins, genes, and/or glycans described herein, and/or binding of IgG, or any combination thereof can be monitored in a subject, and as the levels or activities increase or decrease, relative to control, the dosage or administration of therapeutic agents may be adjusted.

### Methods of Treatment

The invention also features a method for treatment of Kawasaki disease in a subject by contacting a biological sample from the subject with one or more binding agents capable of specifically binding one or more biomarkers (e.g., one or more proteins of Table 6); determining if the level in said biological sample is changed relative to a control value; predicting a response to a Kawasaki disease therapy in said subject based on the level of one or more of said biomarkers; and, if the prediction is positive, administering a Kawasaki disease therapy.

The methods can also be used to determine the proper dosage (e.g., the therapeutically effective amount) of a therapeutic agent for the subject, the proper type of therapeutic agent, or whether a therapy should be administered.

Several therapeutic agents have been used in the treatment of Kawasaki disease. These include, without limitation, administration of IVIG (i.e., IVIG therapy); salicylates (e.g., aspirin); corticosteroids (e.g., prednisone); IL-1 receptor antagonists; anticoagulants (e.g., enoxaparin and/or clopidogrel); infliximab; or any combination thereof. Approximately 25% of subjects do not respond to IVIG treatment. These predisposed subjects are at a higher risk for developing cardiac artery aneurysms or stenosis. These subjects, as classified herein, may be administered low molecular weight heparin (LMWH), such as enoxaparin, in addition to IVIG, for an extended period of time, e.g., weeks to months. Other therapies that may be administered in addition to IVIG, include, but are not limited to, anti-TNF agents (e.g., adalimumab, infliximab, or etanercept); anti-IL treatment (e.g., anti-IL-1a, IL-1b, IL-1RA); statins (e.g., atorvastatin, pravastatin); corticosteroids (e.g., prednisolone, methylprednisolone); immunomodulators (e.g., cyclosporine A, methotrexate), anti-CD20 therapy (e.g., rituximab); plasma exchange, warfarin, and fibrinogen receptor glycoprotein IIb/IIIa, such as Abciximab.

### Diagnostic Kits

The invention also provides test kits and devices. For example, a diagnostic test kit can include one or more binding agents and, if desired, components for detecting and/or evaluating binding between the binding agent and a biomarker. For detection, one or more of binding agents may be labeled. In further embodiments, one or more of the binding agents may be substrate-bound, such that a biomarker-antibody interaction can be established by determining the amount of label attached to the substrate following binding between the antibody and the biomarker. A conventional ELISA is a common, art-known method for detecting antibody-substrate interaction and can be provided as the kit of the invention.

A kit that determines an alteration in the level of a protein, binding of IgG, to a peptide, gene, and/or glycan relative to a reference, such as the level present in a normal control, is useful as a diagnostic kit in the methods of the invention. Such a kit or device may further include a reference sample or standard curve indicative of a positive reference or a normal control reference.

Desirably, the kit will contain instructions for the use of the kit. In one example, the kit contains instructions for the use of the kit for the diagnosis of Kawasaki disease. In yet another example, the kit contains instructions for the use of the kit to monitor therapeutic treatment or dosage regimens. In yet another example, the kit contains instructions for the use of the kit to predict outcome, response to therapy, or disease recurrence. In a further example, the instructions include one or more metrics (e.g., metrics to be used as references).

### EXAMPLES

The following examples are intended to illustrate the invention. They are not meant to limit the invention in any way.

### Methods

Plasma Proteomics

Proteomics on human plasma samples were carried out as follows. In order to enhance sensitivity for less abundant species, the top 14 most abundant proteins were depleted from plasma samples prior to proteomics analysis using the Agilent MARS column. Briefly, 50 μl of human plasma was spin filtered and diluted with the sample buffer and applied to a MARS column. Two fractions, eluted from the column, were collected: fraction 1 representing less abundant proteins (LA Frn1) and fraction 2 representing highly abundant serum proteins (HA Frn2). Both fractions were concentrated and buffer exchanged. LA Frn1 was digested by trypsin, followed by proteomics analysis. Lower abundant fraction-1 tryptic digests were subjected to nano-LC-MS/MS analysis. Separations were carried out using an Ultimate3000 RSLCnano system. Chromatography was carried using analytical EASY-Spray PepMap RSLC, 25 cm×75 μm id, C18, 2 μm and 100 Å nano column thermostatically controlled at 50° C. and at 300 nL/min with a linear gradient from 1% to 38% acetonitrile/water both containing 0.1% (v/v) FA for a total duration of 150 minutes. The separation step was followed by a 30 minute washing step with 99% acetonitrile/water followed by a 20 minute equilibration step with 99% water/acetonitrile both containing 0.1% (v/v) FA. 1.0 μg of each sample was injected into the column. Data dependent MS-MS was performed on the top 25 precursor ions from the full MS scan on the Orbi-Velos MS instrument.

1 μg protein from each sample was injected into the nano-LC-MS/MS system. The data quality was reproducible across all samples. Proteome discoverer with Sequest search engine was used for database searching of each sample against Uniprot human protein database. ˜450 proteins were identified from each sample. The distribution of peptide spectral matches (PSM) was plotted for each sample, and the overall assessment indicated that most samples contained equivalent numbers of PSM as observed.

Overall, ca. 440 protein signatures were acquired per sample using the set criteria.

Log ratios were calculated from the average normalized spectral counts from pairwise comparisons to get the fold change of proteins across the groups. T-test was conducted to determine the significance of the change. Further, the data were filtered to include changes with less than or equal to 0.05 T-test value.

Soluble Plasma Proteins

The concentrations of plasma IgG and IgA were determined using the Total Human IgG Immunoenzymetric Assay and the Human Immunoglobulin A Immunoenzymetric Assay from Cygnus Technologies (Southport, N.C.). The Human FcγR3B ELISA Kit from MyBioSource (San Diego, Calif.) was used to quantify the concentration of soluble CD16 in the patient samples. The concentration of α2,6-sialyltransferase (ST6GALI) in the plasma samples was quantified using the α2,6-Sialyltransferase Assay Kit from Immuno-Biological Laboratories (Minneapolis, Minn.). The plasma samples were diluted in diluents provided with each ELISA. The manufacturer's instructions supplied with the assay kits were followed for the quantitation of each analyte. Absorbance readings for all assays were determined using a SpectraMax M2 spectrophotometer (Molecular Devices, Sunnyvale, Calif.) and analysis was performed using SoftMax Pro 5.2 software (Molecular Devices).

IgG and IgA Glycomics

Methods for glycosylation analysis of Ig proteins is known in the art, for example, as described in Pucic et al, Molecular and Cellular Proteomics, 2011 Oct(10): 1-15.

Tryptic digests of Protein G enriched plasma fraction were subjected to nano-LC-MS/MS analysis. Two IVIg samples were used along with the KD samples as controls throughout the sample prep and data analysis. All separations were carried out using an Ultimate3000 RSLCnano system. Chromatography was carried using analytical EASY-Spray PepMap RSLC, 25 cm×75 μm id, C18, 2 μm and 100 Å nano column thermostatically controlled at 50° C. and at 300 nL/min with a linear gradient from 1% to 38% acetonitrile/water both containing 0.1% (v/v) FA for a total duration of 70 minutes. The separation step was followed by a 30 minutes washing step with 99% acetonitrile/water followed by a 20 minutes equilibration step with 99% water/acetonitrile both containing 0.1% (v/v) FA. 1.0 ug of each sample was injected on column. Data dependent MS-MS was performed on top 25 precursor ions from the full MS scan on the Orbi-Velos MS instrument.

Data analysis was performed using Xcalibur software-Qual and Quan browser. Area of each of the glycopeptides (IgG1, IgG2, IgG3/4 and IgA) was extracted from the base peak chromatogram. All the glycopeptide species m/z and retention time were captured in a processing method that was used for quantitation across all the samples. Peak integration was manually checked for accuracy in the quan browser. The raw abundances/area of all the glycopeptides for all the 30 KD samples and two IVIg controls was then exported in excel. The data was normalized based on the total glycopeptide abundance for each specific IgG (1,2, 3/4) and IgA protein within the sample. Based on these data analysis, we reported the relative abundance of each of the IgG1, IgG2, IgG3/4, and IgA glycopeptide species per sample. Up to this point the samples were all blinded. Once we had the measurement values, then for interpretation we used the unblinded sample description wherein we could group the samples as Febrile controls, Acute phase KD, IVIg treated, and LMWH treated patient samples.

Total Plasma Glycome

To low- and high-abundant protein plasma fractions (25 μL) were added: 2 μL of sample buffer (0.625 mL of 0.5M Tris, pH 6.6, 1 mL of 10% SDS, and 3.375 mL of water), 2 μL of water, and 1 μL of 0.5M dithiothreitol (DTT), followed by incubation at 65° C. for 15 min. The samples were then alkylated by adding 1 μL of 100 mM iodoacetamide and incubated for 30 min in the dark at room temperature. The samples were then set into gel blocks by adding 22.5 μL of 30% (w/w) acrylamide/0.8% (w/v) bis-acrylamide stock solution (37.5:1.0, Protogel, National Diagnostics, Hessle, Hull, UK), 11.25 μL of 1.5M Tris (pH 8.8), 1 μL of 10% SDS, 1 μL of 10% ammonium peroxodisulfate (APS), and finally 1 μL of N,N,N,N′-tetramethyl-ethylenediamine (TEMED), mixed, and then left to set. The gel blocks were transferred to a filter plate and washed with 1 mL of acetonitrile with vortexing on a plate mixer (Sarstedt, Leicester, UK) for 10 min, followed by removal of the liquid. Washing procedure was repeated twice with 1 mL of 20 mM NaHCO3 (pH 7.0) followed by 1 mL of acetonitrile. N-glycans were released by adding 50 μL of 0.1 U/mL PNGaseF (Prozyrne, CA, USA) in 20 mM NaHCO₃ (pH 7.0) to each sample and incubating overnight at 37° C. The released glycans were collected by washing the gel pieces with 3×200 μL of water, 200 μL of acetonitrile, 200 μL of water, and finally 200 μL of acetonitrile. The released glycans were dried, 20 μL of 1% formic acid was added, and the mixture was incubated at room temperature for 40 min and then re-dried. Samples were labeled by adding 5 μL of 2AB labeling solution, vortexed, incubated for 30 min at 65° C., vortexed again, and incubated for a further 90 min. Excess 2AB was removed using Whatman 3MM chromatography paper cleanup: 1-cm square pieces of prewashed, dried Whatman 3MM chromatography paper were folded into quarters and placed into a filter plate (Whatman protein precipitation plate prewashed with 200 μL of acetonitrile followed by 200 μL of water). The 5 μL of 2AB-labeled samples were applied to the paper and left to dry/bind for 15 min. The excess 2AB was washed off the paper by vortexing with 1.8 mL of acetonitrile for 15 min and then removing the acetonitrile using a vacuum manifold; this procedure was repeated four times. The labeled glycans were eluted from the paper by vortexing with 900 μL of water for 30 min and then collected by vacuum into a 2-mL 96-well plate. This was repeated with a further 900 μL of water. The eluates were dried and re-constituted in H₂O/ACN (v/v 30/70). Released and labeled glycans were subsequently fractionated by normal phase chromatography. Ultra Performance Hydrophilic interaction liquid chromatography (UPLC-HILIC) was carried out on a 1.7 μm Waters BEH Glycan (150 mm×2.1 mm) column as detailed in Mittermayr et al. “Multiplexed analytical glycomics: Rapid and confident IgG N-glycan structural elucidation,” J. Prot. Res. 2011:10:3820-9 with retention times expressed as glucose units (GU) with the following conditions: Solvent A was 50 mM formic acid adjusted to pH 4.4 with ammonia solution. Solvent B was acetonitrile. The column temperature was set to 30° C. The 30 min method was used with a linear gradient of 30-47% with solvent A (=70-53% solvent B) at 0.56 mL/min for 23 min followed by 47-70% solvent A and finally reverting back to 30% solvent A to complete the run method. Samples were injected in 60% acetonitrile. Samples were run once and peaks were identified by their GU values which were compared with the GlycoBase 3.1 structural library for serum N-Glycans (http://glycobase.nibrt.ei).

Glycan HILIC data represent the relative percentage areas from HILIC profiles. Therefore, the data are compositional, and convey the relative amounts of glycan structures in a sample rather than the absolute quantities. Compositional data are subject to an awkward constant sum constraint, that is, the values sum to a constant value such as one or one hundred percent. For this reason, the logit transform was used to map the data onto real space.

Gene Expression

RNA was purified from whole blood collected in Paxgene tubes. Reverse transcription was performed on 0.5 μg of RNA with random hexamer priming (Invitrogen) and reverse transcriptase (New England Biolabs). Expression analysis of 48 genes was performed by Real Time-Quantitative Polymerase Chain Reaction (RT-qPCR) performed on the Roche LightCycler 480 II. The ΔΔCp method (Pfaffl 2001) was used to quantify transcripts using the average Cp value of housekeeping genes ACTB, GUSB and RPS14 to normalize each sample. −ΔCp was used as an arbitrary unit measure of transcript quantity.

Study Design

Subject samples selected for this study were between 2 to 33 months of age, Hispanic, males treated for Kawasaki Disease at Kawasaki Disease Center at Rady Children's Hospital.

Thirty subject samples, consisting of blood plasma, DNA, and RNA were used in the study. The Kawasaki Disease group consisted of 3 subjects that received IVIG and LOVENOX® treatment (Note: Subject 1 also received a second dose of IVIG 14 days after the first dose); and 5 subjects that received IVIG. A control group was selected consisting of 5 age and sex matched non-Kawasaki, febrile infants.

Samples were categorized according to the stage of disease or treatment into:

Group A—acute Kawasaki

Group B—sub-acute, post IVIG

Group C—on LOVENOX®;

Group D—convalescent

Febrile controls have only acute samples available.

All samples were randomized and blinded for each analytical platform that was applied.

The subject's clinical information, including treatment information, is provided in Table 21.

TABLE 21
Subject clinical information

| Subject | CA | | |
|---|---|---|---|
| ID# | status* | Treatment** | |
| 1 | 2 | IVIG × 2 | Second IVIG given 2 weeks after initial |
| | | | treatment for cardiac indications |
| 2 | 4 | IVIG × 2, | Second IVIG + infliximab for cardiac |
| | | infliximab | indications |
| 3 | 2 | IVIG × 2 | Second IVIG for cardiac indications |
| 4 | 2 | IVIG × 2 | Second IVIG for treatment resistance |
| 5 | 3 | IVIG × 2 | Second IVIG for treatment resistance |
| 6 | 2 | IVIG × 2 | Second IVIG for cardiac indications |
| 7 | 1 | IVIG × 2 | Second IVIG for treatment resistance |
| 8 | 3 | IVIG × 2 | Second IVIG for treatment resistance |

*Coronary artery status: 1 = normal echo, 2 = coronary artery aneurysms, 3+ transiently dilated, 4 = giant aneurysms
**Subjects treated with second IVIG for “treatment resistance” had fever ≧36 h post-end of IVIG infusion; subjects treated with second IVIG for cardiac indications had abnormal echo and therefore received 2 infusions of IVIG to maximize the anti-inflammatory benefit

The sample information is provided in Table 22.

TABLE 22
Sample information

| Sample | | | | Age |
|---|---|---|---|---|
| ID# | Stage | Day | Treatment | (months) |
| 1A | Acute KD | 4 | IVIG/LMWH | 2.4 |
| 1B | Post IVIG | 12 | | |
| 1C | on Lovenox | 21 | | |
| 1D | Convalescent | 693 | | |
| 2A | Acute KD | 8 | IVIG/LMWH | 3.9 |
| 2B | Post IVIG | 4 | | |
| 2C | on Lovenox | 13 | | |
| 2D | Convalescent | NA | | |
| 3A | Acute KD | | IVIG/LMWH | 14.6 |
| 3B | Post IVIG | NA | | |
| 3C | on Lovenox | 37 | | |
| 3D | Convalescent | 364 | | |
| 4A | Acute KD | | IVIG | 3.4 |
| 4B | Post IVIG | 6 | | |
| 4D | Convalescent | 26 | | |
| 5A | Acute KD | | IVIG | 2.4 |
| 5B | Post IVIG | 16 | | |
| 5D | Convalescent | 33 | | |
| 6A | Acute KD | | IVIG | 15.2 |
| 6B | Post IVIG | 12 | | |
| 6D | Convalescent | 21 | | |
| 7A | Acute KD | | IVIG | 19.6 |
| 7B | Post IVIG | 21 | | |
| 7D | Convalescent | 490 | | |
| 8A | Acute KD | | IVIG | 33.3 |
| 8B | Post IVIG | 15 | | |
| 8D | Convalescent | 22 | | |
| 9A | Acute | NA | Control | |
| 10A | Acute | | Control | |
| 11A | Acute | | Control | |
| 12A | Acute | | Control | |
| 13A | Acute | | Control | |

High risk subjects 1-3 received IVIG/LMWH treatment.
Subjects 4-8 received IVIG treatment leading to disease resolution.
Subjects 9-13 were febrile control.

### Example 1

### Identification of Proteins Indicative of Kawasaki Disease

Shot-gun proteomics identified ˜450 unique proteins on average per sample. In the targeted comparison of Kawasaki disease subjects at the acute stage (1A-8A) with the febrile control (9A-13A), 39 proteins were differentially expressed with 22 down-regulated and 17 up-regulated with a p-value <0.05 as a filter. These differentially expressed proteins belong to: inflammatory response pathway (S100A9, ORM1 ↑), Statin pathway (Apolipoproteins), Complement/Coagulation cascades (CFH, Serpin A1, C1), and Autophagy (GSN ↑), and they were among those altered in Kawasaki compared to the Febrile control.

A list of proteins that were found to be significantly different between the Kawasaki disease group (1A-8A) and the febrile control group (9A-13A) are shown in Table 23.

TABLE 23
Proteins that are significantly different (p < 0.05) in KD group versus febrile control

| | | | Febrile | Acute | |
|---|---|---|---|---|---|
| Accession | | | Control | KD | Log2FC |
| # | Description | p-value | Value | Value | Acute |
| E7ENL6 | collagen, type VI, alpha 3 | 0.0140 | 5.41 | 1.27 | −2.1 |
| Q5MNV8 | F-box protein 47 | 0.0100 | 3.71 | 0.98 | −1.9 |
| P12109 | collagen, type VI, alpha 1 | 0.0161 | 2.70 | 0.76 | −1.8 |
| P10275 | androgen receptor | 0.0018 | 4.01 | | 1.30 |
| Q12888 | tumor protein p53 binding | 0.0003 | 2.01 | 0.76 | −1.4 |
| | protein 1 | | | | |
| Q6UXB8 | peptidase inhibitor 16 | 0.0333 | 10.67 | 4.14 | −1.4 |
| P55290 | cadherin 13 | 0.0017 | 2.13 | 0.83 | −1.4 |
| G5E9A9 | tenascin XB | 0.0308 | 8.19 | 3.50 | −1.2 |
| B0UYX3 | tenascin XB | 0.0365 | 7.42 | 3.20 | −1.2 |
| Q07869 | peroxisome proliferator- | 0.0377 | 1.55 | 0.69 | −1.2 |
| | activated receptor alpha | | | | |
| P02452 | collagen, type I, alpha 1 | 0.0168 | 3.57 | 1.75 | −1.0 |
| P01877 | Ig alpha-2 chain C region | 0.0485 | 9.80 | 4.96 | −1.0 |
| | (A2m marker) | | | | |
| P01765 | Ig heavy chain V-III region TIL | 0.0111 | 1.34 | 0.69 | −1.0 |
| P01876 | Ig alpha-1 chain C region | 0.0420 | 17.95 | 9.19 | −1.0 |
| Q6IMJ5 | olfactomedin 1 | 0.0311 | 3.36 | 1.74 | −1.0 |
| Q5TFM2 | complement factor H | 0.0026 | 92.22 | 48.12 | −0.9 |
| P41222 | prostaglandin D2 synthase | 0.0211 | 2.45 | 1.30 | −0.9 |
| | 21 kDa (brain) | | | | |
| I3L145 | sex hormone-binding globulin | 0.0007 | 12.83 | 6.97 | −0.9 |
| P01593 | Ig kappa chain V-I region | 0.0131 | 3.02 | 1.66 | −0.9 |
| P06396 | gelsolin | 0.0003 | 70.33 | 39.42 | −0.8 |
| P0CG04 | Ig lambda-1 chain C regions | 0.0102 | 12.65 | 7.15 | −0.8 |
| | (Mcg marker) | | | | |
| Q9NSY1 | BMP2 inducible kinase | 0.0130 | 1.34 | 0.76 | −0.8 |
| P01008 | serpin peptidase inhibitor, | 0.0250 | 215.36 | 124.42 | −0.8 |
| | clade C (antithrombin), | | | | |
| | member 1 | | | | |
| P20742 | pregnancy-zone protein | 0.0430 | 10.65 | 6.20 | −0.8 |
| P19827 | inter-alpha-trypsin inhibitor | 0.0447 | 193.28 | 115.09 | −0.7 |
| | heavy chain 1 | | | | |
| P01617 | Ig kappa chain V-II region | 0.0202 | 1.90 | 1.14 | −0.7 |
| P19823 | inter-alpha-trypsin inhibitor | 0.0437 | 186.02 | 114.81 | −0.7 |
| | heavy chain 2 | | | | |
| P08603 | complement factor H | 0.0046 | 226.25 | 141.04 | −0.7 |
| Q04756 | Hepatocyte growth factor | 0.0203 | 6.81 | 4.42 | −0.6 |
| | activator | | | | |
| O75882 | attractin | 0.0433 | 13.74 | 8.92 | −0.6 |
| P01042 | kininogen 1 | 0.0134 | 97.64 | 63.38 | −0.6 |
| P05160 | coagulation factor XIII, B | 0.0401 | 15.43 | 10.20 | −0.6 |
| | polypeptide | | | | |
| B4E1Z4 | complement factor B | 0.0052 | 213.26 | 149.14 | −0.5 |
| H0YFH3 | complement component 1, r | 0.0079 | 33.02 | 23.48 | −0.5 |
| | subcomponent | | | | |
| P00736 | complement component 1, r | 0.0109 | 36.72 | 27.34 | −0.4 |
| | subcomponent | | | | |
| Q16610 | extracellular matrix protein 1 | 0.0263 | 20.87 | 15.70 | −0.4 |
| P09871 | complement component 1, s | 0.0085 | 40.93 | 31.31 | −0.4 |
| | subcomponent | | | | |
| C9JEX1 | kininogen 1 | 0.0195 | 65.08 | 50.06 | −0.4 |
| P13671 | complement component 6 | 0.0163 | 59.56 | 46.05 | −0.4 |
| P02774 | vitamin D binding protein | 0.0259 | 396.66 | 320.83 | −0.3 |
| | (group-specific component) | | | | |
| P01011 | Alpha-1-antichymotrypsin(serpin | 0.0029 | 387.52 | 536.07 | 0.5 |
| | peptidase inhibitor, clade A, | | | | |
| | member 3) | | | | |
| F5H5G7 | lactate dehydrogenase C | 0.0106 | 0.90 | 1.36 | 0.6 |
| G3V3A0 | Alpha-1-antichymotrypsin(serpin | 0.0006 | 152.86 | 246.65 | 0.7 |
| | peptidase inhibitor, clade A, | | | | |
| | member 3) | | | | |
| Q8NEN0 | armadillo repeat containing 2 | 0.0193 | 1.00 | 1.69 | 0.8 |
| Q9H3U1 | unc-45 homolog A (C. elegans) | 0.0145 | 0.88 | 1.52 | 0.8 |
| P59665 | defensin, alpha 1 | 0.0048 | 1.01 | 1.75 | 0.8 |
| P06702 | S100 calcium binding protein A9 | 0.0358 | 6.13 | 12.50 | 1.0 |
| P40879 | solute carrier family 26 (anion | 0.0467 | 1.13 | 2.38 | 1.1 |
| | exchanger), member 3 | | | | |
| P43403 | zeta-chain (TCR) associated | 0.0094 | 1.01 | 2.15 | 1.1 |
| | protein kinase 70 kDa | | | | |
| Q9H2Y7 | Zinc finger protein 106 homolog | 0.0093 | 0.78 | 1.76 | 1.2 |
| P04114 | apolipoprotein B | 0.0021 | 159.94 | 363.53 | 1.2 |
| J3QLI2 | Zinc finger protein 161 homolog | 0.0013 | 1.55 | 3.59 | 1.2 |
| O95445 | apolipoprotein M | 0.0095 | 0.79 | 1.84 | 1.2 |
| P02655 | apolipoprotein C-II | 0.0446 | 1.66 | 4.20 | 1.3 |
| F5GXS5 | apolipoprotein F | 0.0145 | 0.67 | 1.82 | 1.4 |
| I3L1Y6 | zinc finger protein 578 | 0.0402 | 0.67 | 2.14 | 1.7 |
| A4UGR9 | xin actin-binding repeat | 0.0001 | 1.00 | 3.49 | 1.8 |
| | containing 2 | | | | |
| P08519 | lipoprotein, Lp(a) | 0.0475 | 0.90 | 3.54 | 2.0 |
| Q8N2F6 | armadillo repeat containing 10 | 0.0069 | 0.56 | 2.54 | 2.2 |
| Q8NGL3 | olfactory receptor, family 5, | 0.0201 | 1.57 | 7.17 | 2.2 |
| | subfamily D, member 14 | | | | |
| P02763 | orosomucoid 1 | 0.0251 | 17.47 | 162.97 | 3.2 |
| P01009 | Alpha-1-antitrypsin (serpin | 0.0374 | 27.65 | 314.28 | 3.5 |
| | peptidase inhibitor, clade A, | | | | |
| | member 1) | | | | |
| 4843 | nitric oxide synthase 2, | 0.0491 | 0.049135 | 0.00028 | 1.8 |
| | inducible (Gene) | | | | |
| 1088 | carcinoembryonic antigen- | 0.0274 | 0.027424 | 0.00684 | 2.2 |
| | related cell adhesion molecule | | | | |
| | 8 (Gene) | | | | |
| 90865 | interleukin 33 (Gene) | 0.0432 | 0.043233 | 0.000154 | 3.8 |
| 3569 | interleukin 6 (interferon, beta | 0.0100 | 0.00164 | 0.091973 | 5.8 |
| | 2) (Gene) | | | | |
| 4312 | matrix metallopeptidase 1 | 0.0115 | 7.84E−05 | 0.124912 | 10.6 |
| | (interstitial collagenase) (Gene) | | | | |

SERPINA3 protein is an example of a measurement that separates Febrile Controls and Acute Kawasaki Disease. SERPINA3 is considered to be an acute phase response protein observed to be increased during certain types of inflammatory response. CRP is another protein that belongs to the acute phase response group. However, CRP does not appear to consistently separate Febrile Controls from Acute Kawasaki Disease subjects. IVIG treatment appears to reduce levels of both of these proteins as evidenced by reduced levels after IVIG treatment.

### Example 2

### Identification of Proteins Indicative of Development of Cardiac Artery Aneurysm or Stenosis

Current standard of care for Kawasaki disease is treatment with IVIG/ASA at the time of diagnosis at the acute stage. As there is no test or diagnostic tool to identify subjects “at risk” for developing cardiac aneurysm at the acute stage, the aneurysm treatment (LOVENOX®) is generally delayed until echocardiogram imaging is provided later in the course of disease, often when a coronary artery aneurysm has already occurred. A test or diagnostic tool that identifies subjects “at risk” earlier could prevent the development of coronary artery aneurysms and the resulting long term effects (e.g., increased risk of heart attack and other cardiovascular events later in life).

To identify proteins indicative of development of cardiac artery aneurysms or stenosis, a group of Kawasaki disease subjects (1A, 2A, 3A) who were subjected to IVIG/LOVENOX® treatment (based on the clinical outcome) was compared with a group of subjects that were treated with IVIG only (4A, 5A, 6A, 7A, 8A). All subjects in the IVIG/LOVENOX® group developed coronary aneurysm. These subjects may be genetically predisposed to developing coronary artery aneurysm even with the appropriate standard of care treatment.

Ten proteins were significantly altered in the high risk group: three were up-regulated (PROC, F11, APOF) and seven down-regulated (CD44, ANKRD26, LAMP2, BCAM, MMRN1, TGFBI, TET2). The differential levels of these proteins in subjects that developed coronary artery aneurysms or stenosis indicates these proteins are useful for selecting or classifying subjects predisposed to coronary artery aneurysms or stenosis. This early selection may allow for treatment with anticoagulants (e.g., enoxaparin and/or clopidogrel) or other therapies (e.g., infliximab, cyclosporine, and/or prednisone) to begin before the development of coronary artery aneurysms or stenosis, rather than in response to their development as is the current practice.

These protein identifiers were mapped to the following functions:
a. Proteins related to autophagy—CD44 and LAMP2
b. Proteins from complement/coagulation cascades—PROC and F11 are lower in IVIG/LOVENOX® group versus IVIG groups, suggesting that these protein markers for wound healing are present in higher expression in the IVIG responders.
c. Cell adhesion related proteins such as BCAM, TGFB1, and MMRN1 show higher expression in the high risk group as compared with the IVIG responders.

A list of proteins that were found to be significantly different between the IVIG/Lovenox® group (1A-3A) and the IVIG only group (4A-8A) are shown in Table 19.

TABLE 24
Proteins that are significantly different (p < 0.05) in IVIG/LOVENOX ® group
versus IVIG only group

| | | IVIG/ | | | |
|---|---|---|---|---|---|
| Accession | | LOVENOX ® | IVIG | Log fold | |
| No | Description | Value | value | change | p-value |
| P00747 | Plasminogen | 43.78 | 60.98 | −0.5 | 0.0408 |
| P04070 | Vitamin K-dependent protein C | 0.99 | 2.81 | −1.5 | 0.0014 |
| P03951 | Coagulation factor XI | 2.17 | 5.25 | −1.3 | 0.0235 |
| F5GXS5 | Apolipoprotein F | 0.99 | 2.32 | −1.2 | 0.0326 |
| F5H5G7 | L-lactate dehydrogenase | 1.60 | 1.22 | 0.4 | 0.0064 |
| E7EPC6 | CD44 antigen | 3.61 | 2.08 | 0.8 | 0.0148 |
| Q9UPS8 | Ankyrin repeat domain-containing | 5.23 | 2.56 | 1.0 | 0.0199 |
| | protein 26 | | | | |
| B4E2S7 | Lysosome-associated membrane | 2.01 | 0.98 | 1.0 | 0.0234 |
| | glycoprotein 2 | | | | |
| P50895 | Basal cell adhesion molecule | 2.39 | 1.10 | 1.1 | 0.0018 |
| Q13201 | Multimerin-1 | 2.84 | 1.23 | 1.2 | 0.0035 |
| G8JLA8 | Transforming growth factor-beta- | 7.12 | 2.94 | 1.3 | 0.0206 |
| | induced protein ig-h3 | | | | |
| E7EQS8 | Methylcytosine dioxygenase TET2 | 2.88 | 0.98 | 1.6 | 0.0067 |
| P01011 | Alpha-1-antichymotrypsin | 610.74 | 491.26 | 0.3 | 0.0110 |
| P01034 | Cystatin-C | 2.62 | 1.96 | 0.4 | 0.0197 |

In an effort to identify further proteins indicative of development of cardiac artery aneurysms or stenosis, a group of Kawasaki disease subjects (1, 2, 3, 4, and 6) who developed aneurysms based on electrocardiogram imaging was compared with a group of subjects that either did not develop aneurysms or were only dilated and not considered to have developed full aneurysms (5, 7, and 8). The subjects that developed aneurysms may be genetically predisposed to developing coronary artery aneurysm even with the appropriate standard of care treatment.

Student's t-test were used to determine if any significant differences were observed between aneurysm and non-aneurysm groups. Of the 647 measurements, 10 and proteins had a p-value less than 0.05 (see Table 25).

TABLE 25
Proteins that are significantly different (p <
0.05) in aneursym group versus non-aneurysm group

| | | | | Log2 FC |
|---|---|---|---|---|
| Gene | | Accession | | no aneurysm/ |
| Symbol | Analytic | Number | P-value | aneurysm |
| HGFAC | PRO | Q04756 | 1.15E−02 | −0.6 |
| BST1 | PRO | Q10588 | 4.23E−02 | −1 |
| AFTPH | PRO | Q6ULP2 | 4.86E−02 | 2 |
| DTHD1 | PRO | Q6ZMT9 | 4.35E−02 | 0.9 |
| MASP2 | PRO | O00187 | 4.27E−02 | −0.6 |
| XIRP2 | PRO | A4UGR9 | 4.35E−02 | 0.6 |
| MTA3 | PRO | E7EV10 | 6.65E−05 | 1.7 |
| hCG_2014417 | PRO | B7Z718 | 3.45E−02 | −1.4 |
| TET2 | PRO | E7EQS8 | 3.21E−02 | 1.4 |
| TIAM1 | PRO | F5GZ53 | 3.95E−02 | −1.6 |

### Example 3

### Identification of Proteins with Different Binding to Plasma IgG Between Samples of Acute Kawasaki Disease and Febrile Control

A 10K random peptide array was tested for IgG reactivities in plasma samples of KD. A total of 68 out of 10,000 peptides were found to have significantly different binding to plasma IgG between acute KD and febrile control (p<0.05).

Using the 68 peptides, an agglomerative hierarchical clustering technique (via Ward's minimum variance method) was used to naturally break the data into a hierarchy of “similar” clusters. Here, the natural break created two clusters. The 68 peptides identified have the sequences of SEQ ID NOs: 1-68, provided in Table 3, supra. The 68 peptides were attached to the microarray by cysteine-serine-glycine linkers. The peptides (including the linker sequence) are listed in Table 26.

TABLE 26
Peptides identified with different IgG binding
between febrile control and Kawasaki disease
acute stage samples

| SEQ ID | | |
|---|---|---|
| NO: | Peptide Sequence | p-value |
| 69 | CSGAKFLGQSTYIAGYHQVD | 7.67E-07 |
| 70 | CSGFWSKMKPSEEYTTFYRD | 0.001 |
| 71 | CSGFDRSDYMSFHLDDNITI | 0.001 |
| 72 | CSGIRIETPYYKDTEDGKYF | 0.001 |
| 73 | CSGLGLLQAITRNSWVDSAF | 0.001 |
| 74 | CSGKHWEFMQFDIGYIYEKF | 0.001 |
| 75 | CSGNIPSNQHATEIQVDGYH | 0.002 |
| 76 | CSGENFEYHLYDSMIGYEVH | 0.002 |
| 77 | CSGERPDPATYFMPGRDDQY | 0.002 |
| 78 | CSGDNAPYYYREEWHKEFNK | 0.003 |
| 79 | CSGYHWDVQNTFYSMLMLPS | 0.003 |
| 80 | CSGGDFSDYAPTLTQKASYG | 0.003 |
| 81 | CSGQYAFHNLDQNGTVFGNR | 0.003 |
| 82 | CSGYVTNMMINMNYSSLSYS | 0.003 |
| 83 | CSGHWVLSDGYREVYSYNSY | 0.003 |
| 84 | CSGAYHSQLYIDYKDTEWFY | 0.004 |
| 85 | CSGDHPYFVIWDRYKPVHTY | 0.004 |
| 86 | CSGHEHPPYLGMTAYELAQD | 0.004 |
| 87 | CSGGQWSGQGYWYDPFDNMK | 0.004 |
| 88 | CSGTTHFLKDRFESTNHDVY | 0.004 |
| 89 | CSGVEDPRVGHSLFQDANYY | 0.004 |
| 90 | CSGDPVQIFNTAEHSGPYIR | 0.004 |
| 91 | CSGHDHFRGGKFILSTQAIW | 0.005 |
| 92 | CSGNYPLPKYYYNWFEPRVW | 0.005 |
| 93 | CSGQYDDPDWQIHYKLEARG | 0.005 |
| 94 | CSGQDPYMDLHYDKNQIEQA | 0.006 |
| 95 | CSGRGPHNFEIAETDAQMIE | 0.006 |
| 96 | CSGDAYTNQDISEEEHMHRY | 0.006 |
| 97 | CSGGALEWIYYAGPKPGYWE | 0.006 |
| 98 | CSGPHSTHQIFYKSYETDMA | 0.007 |
| 99 | CSGIYWGPMSTGHLPSQAQF | 0.007 |
| 100 | CSGYTHFWWLDKYMRYEVAT | 0.007 |
| 101 | CSGFLTEYYEYQNNLFHAFR | 0.007 |
| 102 | CSGSRGDAAAWGILFDANWK | 0.008 |
| 103 | CSGAMLGGMWAAYYPFPVPG | 0.008 |
| 104 | CSGYYWATGPEGPFRHPGAR | 0.008 |
| 105 | CSGDMEFTVFDIDMEKHYKY | 0.008 |
| 106 | CSGHYWDYQQLGFQGHLDHR | 0.009 |
| 107 | CSGDQHFVWGPTGRAPMNYG | 0.009 |
| 108 | CSGTNGFHIPFYSDFQSAAA | 0.009 |
| 109 | CSGMKYDVWKFYNGDDMRVS | 0.009 |
| 110 | CSGWYDNMYKTGFYRMYLLT | 0.009 |
| 111 | CSGEFQDYNHSDVNMSNHPY | 0.009 |
| 112 | CSGYIPEQQPHEADNLYKDA | 0.010 |
| 113 | CSGFYTLPHRPLYYYGYVAS | 0.010 |
| 114 | CSGHRWLEEANTEYMTMNSI | 0.010 |
| 115 | CSGGPINAYQKQDYSIEPEH | 0.010 |
| 116 | CSGAGDQYVRIDKSTRISNI | 0.011 |
| 117 | CSGSMKTVEADWYTYEPWWH | 0.011 |
| 118 | CSGNHIYIVQTAYGVTGETS | 0.011 |
| 119 | CSGMFAYHRAWPVWSSVLHV | 0.012 |
| 120 | CSGWPNPYFYHKKDTYWAHY | 0.012 |
| 121 | CSGNYDHLLGQYPIRNWWSL | 0.012 |
| 122 | CSGESMDVVWPYGYKFTQYW | 0.012 |
| 123 | CSGTHMEHDFHIPLEMYKYM | 0.012 |
| 124 | CSGVTPLEIIEQIREHLDIK | 0.013 |
| 125 | CSGLQKPFDYYMKEWQVDNE | 0.013 |
| 126 | CSGVMQRWPENHFLQTHYDD | 0.013 |
| 127 | CSGWWNWRSAYKEGDVAYPS | 0.013 |
| 128 | CSGGWVLDDWSSHHINYYIE | 0.013 |
| 129 | CSGFSPHYQYVGLFPYVKYI | 0.013 |
| 130 | CSGANLVYMWGSAVHTSDPQ | 0.013 |
| 131 | CSGSPVMYSIANYKYQTMHL | 0.013 |
| 132 | CSGDQEISYLNSHTDLFVGR | 0.014 |
| 133 | CSGYEPTDVYLTYRKLATKD | 0.014 |
| 134 | CSGPVPQERTDLFTGAHRAL | 0.014 |
| 135 | CSGREVYHEIKSGRAIEIYM | 0.014 |
| 136 | CSGHDAIWYDWNPYPSKHES | 0.014 |

### Example 4

### Identification of Biomarkers Indicative of Development of Cardiac Artery Aneurysm or Stenosis

The predisposition for development of aneurysms can also be examined by looking at the difference in gene expression between the aneurysm group (Subjects 1 and 2) and the non-aneurysm group (Subjects 3-8). Measurements that show persistently different values in the Sub-acute samples in Subjects 1 and 2 were identified by selecting those measurements where the absolute value of the standard normal deviate was greater than 2.58 for both subjects. (Z=2.58 is the critical value for the two-tailed 99% confidence interval of the normal distribution). Sixty three measurements were observed to be persistently perturbed, either up or down, in subjects 1 and 2 at the sub-acute phase as shown in Table 27.

TABLE 27
Biomarkers that are differentially expressed in aneursym group versus non-aneurysm
group after treatment with IVIG

| | | | | | | | Sub- | | | |
|---|---|---|---|---|---|---|---|---|---|---|
| | | | | Acute | Sub- | Sub- | Acute | | | Conv. |
| | | Acute | Acute | Subj. | acute | acute | Subj. | Conv. | Conv. | Subj. |
| Measurement | Anal. | Subj. 1 | Subj. 2 | 3-8 | Subj. 1 | Subj. 2 | 3-8 | Subj. 1 | Subj. 2 | 3-8 |
| ACTB | PRO | 3.46 | 5.74 | 2.67 | 2.67 | 3.84 | 0.82 | 5.74 | 2.80 | 0.0 |
| AFM | PRO | −3.35 | −3.47 | 4.03 | −3.41 | −3.22 | 0.70 | −2.42 | −0.46 | 0.0 |
| AHSG | PRO | −2.86 | −2.63 | 2.63 | −3.27 | −4.57 | 1.42 | −4.96 | −1.66 | 0.0 |
| AR | PRO | −10.28 | −3.67 | −1.38 | −9.48 | −5.43 | −2.42 | 0.30 | −2.31 | 0.0 |
| BCHE | PRO | −0.30 | −4.10 | −1.58 | −4.66 | −4.19 | −0.36 | −3.38 | −1.89 | 0.0 |
| BTD | PRO | −3.87 | −1.67 | 1.99 | −3.10 | −3.19 | 2.02 | −2.04 | 0.62 | 0.0 |
| C9 | PRO | 4.49 | 3.84 | 4.15 | 4.18 | 3.04 | 1.31 | 3.30 | 1.97 | 0.0 |
| CD80 | GE | −5.03 | −5.08 | −1.53 | −4.33 | −5.78 | −0.98 | −0.38 | NA | 0.0 |
| CDH13 | PRO | −2.02 | −4.53 | 9.51 | −4.41 | −4.89 | −0.98 | −1.82 | 0.78 | 0.0 |
| CEACAM8 | GE | 4.98 | 8.87 | −1.69 | 3.80 | 3.60 | −0.45 | 4.51 | NA | 0.0 |
| CHL1 | PRO | −1.14 | −1.43 | −0.24 | −2.82 | −2.78 | 0.36 | −2.05 | −2.11 | 0.0 |
| CRP | PRO | 4.65 | 4.17 | 2.89 | 3.30 | 3.08 | −0.62 | 2.24 | 1.73 | 0.0 |
| CTSD | PRO | 12.48 | 12.56 | −0.22 | 11.18 | 10.87 | 2.05 | −0.18 | 11.43 | 0.0 |
| F10 | PRO | 1.09 | 1.23 | 4.05 | −3.20 | −3.35 | 0.72 | −1.31 | 0.51 | 0.0 |
| F9 | PRO | 2.74 | 0.54 | 3.42 | −4.92 | −6.45 | 1.32 | −0.99 | 2.45 | 0.0 |
| GP12LA | GLY | −4.68 | −5.39 | −5.13 | −3.96 | −8.08 | 0.52 | −3.08 | NA | 0.0 |
| GP13HA | GLY | −11.39 | −11.40 | −6.67 | −7.83 | −6.26 | 2.76 | −5.28 | NA | 0.0 |
| GP14HA | GLY | −0.93 | −1.81 | −2.63 | −2.86 | −3.30 | 1.99 | −1.10 | NA | 0.0 |
| GP14LA | GLY | −4.56 | −5.34 | −2.08 | −2.78 | −3.90 | 0.23 | −1.44 | NA | 0.0 |
| GP16LA | GLY | −4.83 | −5.61 | −3.35 | −9.05 | −5.29 | 0.14 | −3.37 | NA | 0.0 |
| GP18HA | GLY | −9.30 | −11.24 | −6.92 | −5.11 | −5.57 | −5.29 | −4.19 | NA | 0.0 |
| GP18LA | GLY | −1.99 | −9.33 | −3.58 | −5.33 | −9.96 | −0.07 | −2.99 | NA | 0.0 |
| GP19LA | GLY | −1.38 | −4.51 | −2.25 | −2.92 | −5.86 | −0.21 | −0.62 | NA | 0.0 |
| GP20HA | GLY | −6.37 | −3.41 | −7.20 | −16.34 | −10.72 | −2.93 | −7.11 | NA | 0.0 |
| GP20LA | GLY | −9.24 | −7.52 | −0.89 | −16.51 | −11.69 | −1.55 | −5.65 | NA | 0.0 |
| GP24LA | GLY | −2.96 | −2.91 | −5.57 | −5.51 | −3.13 | −0.81 | −0.75 | NA | 0.0 |
| GP29LA | GLY | −2.36 | 1.15 | −5.36 | −6.00 | 3.84 | −1.62 | −0.95 | NA | 0.0 |
| GP32HA | GLY | −4.04 | −3.19 | −1.63 | −6.21 | −5.19 | −0.38 | −0.34 | NA | 0.0 |
| GP32LA | GLY | −3.04 | −4.29 | −1.70 | −4.11 | −4.48 | 0.29 | −1.29 | NA | 0.0 |
| GP34HA | GLY | −2.44 | −2.13 | 2.25 | −4.35 | −3.66 | −2.22 | 0.46 | NA | 0.0 |
| GP34LA | GLY | −2.58 | −3.32 | −3.65 | −3.32 | −3.58 | −0.18 | −0.45 | NA | 0.0 |
| GP37HA | GLY | 2.57 | 3.33 | −1.24 | 2.72 | 3.23 | −0.59 | 2.47 | NA | 0.0 |
| GP38LA | GLY | −1.61 | −2.65 | −5.11 | −4.93 | 3.33 | −0.91 | 0.78 | NA | 0.0 |
| GP3LA | GLY | −5.97 | −3.02 | −4.26 | −3.01 | −4.92 | 0.74 | −4.69 | NA | 0.0 |
| GP42HA | GLY | 3.26 | 4.01 | −1.60 | 2.69 | 3.69 | −0.15 | 2.67 | NA | 0.0 |
| GP43HA | GLY | 5.61 | 6.14 | 0.12 | 5.85 | 6.18 | −0.34 | 3.13 | NA | 0.0 |
| GP43LA | GLY | 3.74 | 4.22 | −5.21 | 3.90 | 4.48 | −0.99 | 2.11 | NA | 0.0 |
| GP44HA | GLY | 7.89 | 9.42 | −0.60 | 8.49 | 9.84 | −0.21 | 3.32 | NA | 0.0 |
| GP44LA | GLY | 3.62 | 5.22 | −7.90 | 3.83 | 5.58 | 5.31 | 1.38 | NA | 0.0 |
| GP9HA | GLY | 4.83 | 5.35 | −4.02 | 3.99 | 5.20 | 0.85 | −2.26 | NA | 0.0 |
| GP9LA | GLY | 2.69 | 6.69 | −9.94 | 4.12 | 6.16 | −0.37 | −2.38 | NA | 0.0 |
| GSN | PRO | −4.66 | −6.50 | 4.79 | −4.15 | −4.87 | 2.93 | −2.77 | −0.87 | 0.0 |
| GUSB | GE | −4.91 | −4.67 | −4.48 | −3.96 | −5.12 | −0.39 | −2.03 | NA | 0.0 |
| HP | PRO | 1.45 | 2.93 | 2.93 | 2.78 | 2.79 | 1.40 | 2.57 | 1.21 | 0.0 |
| HPR | PRO | 1.91 | 3.90 | 2.36 | 3.79 | 3.72 | 1.23 | 3.46 | 0.97 | 0.0 |
| IgA | ELISA | 0.26 | 10.99 | −2.98 | 3.88 | 11.39 | −1.47 | 7.17 | NA | 0.0 |
| LRG1 | PRO | 6.05 | 6.39 | 3.40 | 3.37 | 3.93 | 0.93 | 2.43 | 3.62 | 0.0 |
| LUM | PRO | −1.79 | −4.21 | 4.43 | −6.10 | −6.03 | 1.65 | −5.21 | −0.78 | 0.0 |
| MMP9 | PRO | 8.18 | 5.57 | −7.51 | 2.96 | 3.82 | −0.42 | 3.29 | NA | 0.0 |
| NID1 | PRO | 0.01 | 4.30 | 4.08 | 2.66 | 4.15 | −0.53 | 0.16 | −1.98 | 0.0 |
| ORM1 | PRO | 13.42 | 16.46 | −0.61 | 9.34 | 13.34 | 0.21 | 1.68 | −1.78 | 0.0 |
| ORM2 | PRO | 5.79 | 13.52 | 2.75 | 6.30 | 6.85 | 2.31 | 4.60 | 0.23 | 0.0 |
| PROC | PRO | −3.03 | −4.13 | 2.73 | −4.21 | −2.81 | −0.36 | −1.64 | −1.00 | 0.0 |
| S100A12 | GE | 9.08 | 6.74 | −1.11 | 3.91 | 5.55 | −1.57 | 4.74 | NA | 0.0 |
| S100A8 | PRO | 3.78 | 4.69 | 3.36 | 3.21 | 3.88 | 0.85 | 3.93 | 2.67 | 0.0 |
| SERPINA4 | PRO | −6.97 | −4.28 | 1.85 | −5.80 | −3.28 | 0.48 | −2.53 | −0.15 | 0.0 |
| ST6GAL1 | ELISA | 2.25 | 5.80 | −3.01 | 3.70 | 6.25 | −1.47 | 4.86 | NA | 0.0 |
| TNXB | PRO | 0.65 | −4.38 | 3.15 | −3.77 | −3.47 | 0.07 | −6.52 | −0.27 | 0.0 |
| TNXB_1 | PRO | 0.37 | −5.16 | 0.45 | −3.54 | −3.90 | 0.83 | −7.82 | −0.62 | 0.0 |
| TTN | PRO | −0.04 | 0.53 | 0.51 | −12.01 | −2.65 | 1.11 | 0.26 | −1.27 | 0.0 |
| VCAN | GE | 2.75 | 3.57 | −1.37 | 4.53 | 3.68 | −0.32 | 4.50 | NA | 0.0 |

PRO, protein from shot-gun proteomics;
ELISA, soluble plasma protein;
GE, gene expression;
GLY, serum protein glycans;
GP, fraction number;
HA, high-abundant fraction;
LA, low abundant fraction.

### Example 5

### Identification of Biomarkers Indicative of Response to IVIG Treatment

Measurements made on samples from KD subjects were analyzed to examine the effect of IVIG treatment. Acute phase samples were compared to samples collected after IVIG treatment.

A total of 690 analytes per sample from 21 biological samples with the complete data sets was used for the analysis. Samples were divided into an “Acute Disease” group and a “Sub-acute” group. Differences between the two groups were examined by Student's t-tests after variance stabilizing transformation. Transformations were performed by analytic: plasma proteomics, log₂; RT-qPCR −ΔCp, none; plasma protein ELISA, none; plasma glycans, log₂; Ig glycopeptides, logit. A total of 157 measurements with P≦0.01 were considered to be significantly different between the two groups (Acute Value vs. Sub-acute Value) and are listed in table 28.

TABLE 28
Biomarkers that are differentially expressed in acute stage versus after treatment with
IVIG

| | | Accession | | | | |
|---|---|---|---|---|---|---|
| | | No/ | | | Acute | Post- |
| | | glycan | | | Stage | treatment |
| Analyte | Analytic | structure | Gene name | p-value | Value | Value |
| CRP | PRO | P02741 | C-reactive protein, | 1.07E−09 | 15.7 | 0.9 |
| | | | pentraxin-related | | | |
| HPR | PRO | P00739 | haptoglobin-related | 6.21E−09 | 107.2 | 9.02 |
| | | | protein | | | |
| ITIH2 | PRO | P19823 | inter-alpha-trypsin | 3.22E−08 | 122.08 | 259.05 |
| | | | inhibitor heavy chain 2 | | | |
| HP | PRO | P00738 | haptoglobin | 3.85E−08 | 249.76 | 29.02 |
| GSN | PRO | P06396 | gelsolin | 5.31E−08 | 41.19 | 73.46 |
| AFM | PRO | P43652 | afamin | 5.46E−08 | 30.22 | 74.24 |
| AMBP | PRO | P02760 | alpha-1- | 2.27E−07 | 31.07 | 59.83 |
| | | | microglobulin/bikunin | | | |
| | | | precursor | | | |
| FCGR1A | GE | 2209 | Fc fragment of IgG, | 2.90E−07 | −0.74 | −3.72 |
| | | | high affinity Ia, receptor | | | |
| | | | (CD64) | | | |
| BTD | PRO | F8W1Q3 | biotinidase | 4.26E−07 | 4.82 | 8.22 |
| SERPINA4 | PRO | P29622 | serpin peptidase | 6.12E−07 | 5.84 | 14.94 |
| | | | inhibitor, clade A | | | |
| | | | (alpha-1 antiproteinase, | | | |
| | | | antitrypsin), member 4 | | | |
| C9 | PRO | P02748 | complement | 6.66E−07 | 50.32 | 32.85 |
| | | | component 9 | | | |
| ORM2 | PRO | P19652 | orosomucoid 2 | 6.85E−07 | 31.78 | 4.93 |
| S100A12 | GE | 6283 | S100 calcium binding | 1.42E−06 | 0.76 | −2.41 |
| | | | protein A12 | | | |
| LUM | PRO | P51884 | lumican | 1.51E−06 | 15.28 | 26.94 |
| CDH13 | PRO | P55290 | cadherin 13 | 2.31E−06 | 0.8 | 1.96 |
| ORM1 | PRO | P02763 | orosomucoid 1 | 3.15E−06 | 159.54 | 8.7 |
| MMP9 | GE | 4318 | matrix metallopeptidase 9 | 3.36E−06 | 4.31 | 2.17 |
| GP13HA | GLY | A2[3]BG1S | | 3.77E−06 | 1.29 | 1.95 |
| | | [3]1 | | | | |
| | | A2[3]BG1S | | | | |
| | | [6]1 | | | | |
| | | M7 D1 | | | | |
| | | FA2[3]G1S | | | | |
| | | [3]1 | | | | |
| CHL1 | PRO | O00533 | cell adhesion molecule | 4.59E−06 | 0.93 | 4.93 |
| | | | L1-like | | | |
| AR | PRO | P10275 | androgen receptor | 6.17E−06 | 1.14 | 3.14 |
| KNG1 | PRO | P01042 | kininogen 1 | 6.94E−06 | 67.17 | 115.34 |
| LRG1 | PRO | P02750 | leucine-rich alpha-2- | 7.32E−06 | 49.06 | 21.97 |
| | | | glycoprotein 1 | | | |
| HGFAC | PRO | Q04756 | HGF activator | 1.62E−05 | 4.17 | 9.41 |
| S100A9 | PRO | P06702 | S100 calcium binding | 2.52E−05 | 13.5 | 2.98 |
| | | | protein A9 | | | |
| ITIH1 | PRO | P19827 | inter-alpha-trypsin | 2.94E−05 | 127.79 | 248.86 |
| | | | inhibitor heavy chain 1 | | | |
| MST1 | PRO | P26927 | macrophage stimulating | 3.10E−05 | 11.58 | 18.42 |
| | | | 1 (hepatocyte growth | | | |
| | | | factor-like) | | | |
| IGFALS | PRO | P35858 | insulin-like growth | 3.49E−05 | 9.52 | 20.4 |
| | | | factor binding protein, | | | |
| | | | acid labile subunit | | | |
| SERPINA3 | PRO | G3V3A0 | serpin peptidase | 3.53E−05 | 227.39 | 124.78 |
| | | | inhibitor, clade A | | | |
| | | | (alpha-1 antiproteinase, | | | |
| | | | antitrypsin), member 3 | | | |
| PGLYRP2 | PRO | Q96PD5 | peptidoglycan | 3.69E−05 | 28.53 | 47.95 |
| | | | recognition protein 2 | | | |
| GP18HA | GLY | FA2G2S[6]1 | | 3.86E−05 | 4.04 | 6.66 |
| GP12LA | GLY | FA2BG2 | | 4.25E−05 | 0.06 | 0.09 |
| TNFRSF1A | GE | 7132 | tumor necrosis factor | 4.69E−05 | −3.89 | −5.19 |
| | | | receptor superfamily, | | | |
| | | | member 1A | | | |
| TNXB | PRO | G5E9A9 | tenascin XB | 5.35E−05 | 3.02 | 9.09 |
| CLU | PRO | P10909 | clusterin | 5.84E−05 | 22.8 | 32.62 |
| AHSG | PRO | P02765 | alpha-2-HS- | 6.16E−05 | 195.41 | 284.02 |
| | | | glycoprotein | | | |
| SERPINA3 | PRO | P01011 | serpin peptidase | 6.35E−05 | 486.42 | 298.31 |
| | | | inhibitor, clade A | | | |
| | | | (alpha-1 antiproteinase, | | | |
| | | | antitrypsin), member 3 | | | |
| LBP | PRO | P18428 | lipopolysaccharide | 6.53E−05 | 10.3 | 4.44 |
| | | | binding protein | | | |
| GPLD1 | PRO | P80108 | glycosylphosphatidylino | 6.79E−05 | 2.89 | 8.13 |
| | | | sitol specific | | | |
| | | | phospholipase D1 | | | |
| TNXB | PRO | B0UYX3 | tenascin XB | 6.79E−05 | 2.69 | 8.64 |
| RBP4 | PRO | Q5VY30 | retinol binding protein | 7.66E−05 | 20.88 | 70.17 |
| | | | 4, plasma | | | |
| FBLN1 | PRO | B1AHL2 | fibulin 1 | 7.67E−05 | 10.65 | 16.87 |
| GP20LA | GLY | A2G2S[3,6]2 | | 7.96E−05 | 4.45 | 6.27 |
| ST6GAL1 | ELISA | NA | ST6 beta- | 8.04E−05 | 33.66 | 11.47 |
| | | | galactosamide alpha- | | | |
| | | | 2,6-sialyltranferase 1 | | | |
| GP18LA | GLY | FA2G2S[6]1 | | 8.97E−05 | 1.25 | 1.58 |
| VCAN | GE | 1462 | versican | 1.02E−04 | −0.85 | −2.62 |
| SERPINC1 | PRO | P01008 | serpin peptidase | 1.15E−04 | 118.4 | 195.9 |
| | | | inhibitor, clade C | | | |
| | | | (antithrombin), member 1 | | | |
| GP12HA | GLY | FA2BG2 | | 1.32E−04 | 0.29 | 0.54 |
| HBA1 | PRO | P69905 | hemoglobin, alpha 1 | 1.35E−04 | 88.8 | 4.91 |
| BCHE | PRO | P06276 | butyrylcholinesterase | 1.42E−04 | 4.48 | 9.23 |
| S100A8 | PRO | P05109 | S100 calcium binding | 1.52E−04 | 4.57 | 2.22 |
| | | | protein A8 | | | |
| HSPA5 | PRO | P11021 | heat shock 70 kDa | 1.75E−04 | 1 | 2.05 |
| | | | protein 5 (glucose- | | | |
| | | | regulated protein, | | | |
| | | | 78 kDa) | | | |
| KLKB1 | PRO | H0YAC1 | kallikrein B, plasma | 2.0E−04 | 13.66 | 21.26 |
| | | | (Fletcher factor) 1 | | | |
| GP41LA | GLY | A4G4S[3,3, | | 2.11E−04 | 0.63 | 0.35 |
| | | 3,3]4 | | | | |
| CSF2RB | GE | 1439 | colony stimulating | 2.15E−04 | −2.41 | −4.16 |
| | | | factor 2 receptor, beta, | | | |
| | | | low-affinity | | | |
| | | | (granulocyte- | | | |
| | | | macrophage) | | | |
| HBD | PRO | P02042 | hemoglobin, delta | 2.45E−04 | 40.53 | 3.68 |
| CFH | PRO | P08603 | complement factor H | 2.55E−04 | 143.84 | 215.03 |
| GP11HA | GLY | FA2G2 | | 2.61E−04 | 2.84 | 4.8 |
| GP19HA | GLY | FA2BG2S | | 3.24E−04 | 0.76 | 1.44 |
| | | [3]1; | | | | |
| | | FA2BG2S | | | | |
| | | [6]1 | | | | |
| GUSB | GE | 2990 | glucuronidase, beta | 3.46E−04 | −5.18 | −4.9 |
| SERPINA1 | PRO | P01009 | serpin peptidase | 3.55E−04 | 264.17 | 21.64 |
| | | | inhibitor, clade A | | | |
| | | | (alpha-1 antiproteinase, | | | |
| | | | antitrypsin), member 1 | | | |
| FCGR3A | ELISA | NA | Fc fragment of IgG, low | 3.74E−04 | 8.95 | 5.12 |
| | | | affinity IIIa, receptor | | | |
| | | | (CD16a) | | | |
| CFH | PRO | Q5TFM2 | complement factor H | 4.03E−04 | 50.22 | 93.38 |
| GP42LA | GLY | A4F1G4S | | 4.1E−04 | 1.2 | 0.5 |
| | | [3,3,3,6]4 | | | | |
| GP42HA | GLY | A4F1G4S | | 4.16E−04 | 1.78 | 0.63 |
| | | [3,3,3,6]4 | | | | |
| GP3HA | GLY | M5; | | 4.22E−04 | 1.26 | 1.99 |
| | | FA2B; | | | | |
| | | A2[6]G1 | | | | |
| CEP70 | PRO | Q8NHQ1 | centrosomal protein | 4.45E−04 | 1.93 | 3.55 |
| | | | 70 kDa | | | |
| GP10HA | GLY | A2BG2 | | 4.49E−04 | 0.03 | 0.06 |
| HBG2 | PRO | P69892 | hemoglobin, gamma G | 4.61E−04 | 18.05 | 1.45 |
| HBG1 | PRO | P69891 | hemoglobin, gamma A | 4.63E−04 | 18.05 | 1.45 |
| GP41HA | GLY | A4G4S[3,3, | | 4.75E−04 | 0.9 | 0.47 |
| | | 3,3]4 | | | | |
| HBB | PRO | P68871 | hemoglobin, beta | 5.42E−04 | 81.49 | 7.7 |
| IL1B | GE | 3553 | interleukin 1, beta | 5.71E−04 | −3.25 | −5.13 |
| GP43LA | GLY | A4G4LacS | | 5.71E−04 | 1.27 | 0.35 |
| | | [3,3,3,6]4 | | | | |
| IL1RN | GE | 3557 | interleukin 1 receptor | 6.60E−04 | −1.59 | −3.34 |
| | | | antagonist | | | |
| F13B | PRO | P05160 | coagulation factor XIII, | 6.85E−04 | 10.83 | 20.03 |
| PRO | | | B polypeptide | | | |
| GP5HA | GLY | FA2[6]G1 | | 7.79E−04 | 2.68 | 4.93 |
| S100A8 | GE | 6279 | S100 calcium binding | 7.99E−04 | 1.03 | −1.14 |
| | | | protein A8 | | | |
| APOH | PRO | P02749 | apolipoprotein H (beta- | 8.13E−04 | 87.26 | 130.48 |
| | | | 2-glycoprotein I) | | | |
| FBLN1 | PRO | P23142 | fibulin 1 | 8.21E−04 | 10.7 | 16.65 |
| GP43HA | GLY | A4G4LacS | | 8.23E−04 | 1.28 | 0.32 |
| | | [3,3,3,6]4 | | | | |
| THBS4 | PRO | P35443 | thrombospondin 4 | 8.79E−04 | 3.84 | 8.92 |
| GP37HA | GLY | A4F1G3S | | 9.41E−04 | 0.48 | 0.25 |
| | | [3,3,3]3 | | | | |
| | | A4F1G3S | | | | |
| | | [3,3,6]3 | | | | |
| | | A4F1G3S | | | | |
| | | [3,6,6]3 | | | | |
| FN1 | PRO | F8W7G7 | fibronectin 1 | 9.44E−04 | 42.92 | 111.05 |
| GP44HA | GLY | A4F3G4S | | 9.51E−04 | 0.9 | 0.24 |
| | | [3,3,3,3]4 | | | | |
| IGF2 | PRO | P01344 | insulin-like growth | 1.01E−03 | 0.87 | 1.77 |
| | | | factor 2 (somatomedin | | | |
| | | | A) | | | |
| S100A11 | GE | 6282 | S100 calcium binding | 1.03E−03 | 2.62 | 1.19 |
| | | | protein A11 | | | |
| FN1 | PRO | P02751 | fibronectin 1 | 1.04E−03 | 47.41 | 120.85 |
| PROC | PRO | P04070 | protein C (inactivator of | 1.06E−03 | 2.07 | 3.68 |
| | | | coagulation factors Va | | | |
| | | | and VIIIa) | | | |
| GP44LA | GLY | A4F3G4S | | 1.15E−03 | 0.47 | 0.11 |
| | | [3,3,3,3]4 | | | | |
| GP6HA | GLY | FA2[3]G1 | | 1.32E−03 | 0.95 | 1.94 |
| GP13LA | GLY | A2[3]BG1S | | 1.41E−03 | 0.43 | 0.66 |
| | | [3]1; | | | | |
| | | A2[3]BG1S | | | | |
| | | [6]1; M7 | | | | |
| | | D1; | | | | |
| | | FA2[3]G1S | | | | |
| | | [3]1 | | | | |
| KNG1 | PRO | C9JEX1 | kininogen 1 | 1.56E−03 | 51.86 | 65.54 |
| GP24LA | GLY | A2BG2S[6, | | 1.63E−03 | 1.66 | 2.03 |
| | | 6]2 | | | | |
| SEPP1 | PRO | P49908 | selenoprotein P, | 1.76E−03 | 2.97 | 4.76 |
| | | | plasma, 1 | | | |
| GP8HA | GLY | FA2[3]BG1; | | 1.85E−03 | 0.81 | 1.12 |
| | | M6 D3; | | | | |
| NRP1 | PRO | O14786 | neuropilin 1 | 1.91E−03 | 1.26 | 2.36 |
| NCAM1 | PRO | E9PLH7 | neural cell adhesion | 1.94E−03 | 2.16 | 3.79 |
| | | | molecule 1 | | | |
| PKDREJ | PRO | Q9NTG1 | polycystin (PKD) family | 1.97E−03 | 2.37 | 0.9 |
| | | | receptor for egg jelly | | | |
| TNFRSF1B | GE | 7133 | tumor necrosis factor | 1.99E−03 | −1.94 | −2.88 |
| | | | receptor superfamily, | | | |
| | | | member 1B | | | |
| KIF20B | PRO | Q96Q89 | kinesin family member | 2.04E−03 | 4.47 | 2.22 |
| | | | 20B | | | |
| LYZ | PRO | P61626 | lysozyme | 2.05E−03 | 1.66 | 0.78 |
| COL1A1 | PRO | P02452 | collagen, type I, alpha 1 | 2.05E−03 | 1.8 | 3.88 |
| GP7HA | GLY | FA2[6]BG1 | | 2.12E−03 | 0.69 | 1.22 |
| GP23HA | GLY | A2G2S[3,6]2 | | 2.4E−03 | 38.79 | 32.23 |
| A1BG | PRO | P04217 | alpha-1-B glycoprotein | 2.47E−03 | 100.11 | 131.28 |
| F13A1 | PRO | P00488 | coagulation factor XIII, | 2.52E−03 | 6.73 | 14.07 |
| | | | A1 polypeptide | | | |
| ACTC1 | PRO | P68032 | actin, alpha, cardiac | 2.6E−03 | 5.8 | 3.25 |
| | | | muscle 1 | | | |
| ECM1 | PRO | Q16610 | extracellular matrix | 2.62E−03 | 14.69 | 19.83 |
| | | | protein 1 | | | |
| GP32LA | GLY | A3G3S[3,3, | | 2.63E−03 | 0.65 | 1.14 |
| | | 6]3 | | | | |
| TNFSF13B | GE | 10673 | tumor necrosis factor | 2.64E−03 | −1.73 | −2.71 |
| | | | (ligand) superfamily, | | | |
| | | | member 13b | | | |
| APOC1 | PRO | P02654 | apolipoprotein C-I | 2.74E−03 | 1.65 | 3.27 |
| GP35HA | GLY | A3F1G3S | | 2.79E−03 | 2.87 | 1.96 |
| | | [3,3,3]3 | | | | |
| CPB2 | PRO | Q96IY4 | carboxypeptidase B2 | 2.79E−03 | 4.11 | 7.78 |
| | | | (plasma) | | | |
| ATRN | PRO | O75882 | attractin | 2.85E−03 | 7.93 | 12.91 |
| GP26HA | GLY | FA2BG2S | | 2.86E−03 | 0.65 | 0.99 |
| | | [3,6]2 | | | | |
| | | FA2BG2S | | | | |
| | | [6,6]2 | | | | |
| ZNF217 | PRO | O75362 | zinc finger protein 217 | 2.89E−03 | 3.95 | 7.72 |
| SHBG | PRO | I3L145 | sex hormone-binding | 2.93E−03 | 8 | 13.57 |
| | | | globulin | | | |
| PZP | PRO | P20742 | pregnancy-zone protein | 3.05E−03 | 7.04 | 16.74 |
| ITIH3 | PRO | Q06033 | inter-alpha-trypsin | 3.20E−03 | 98.25 | 59.4 |
| | | | inhibitor heavy chain 3 | | | |
| COL11A2 | PRO | H0YHY3 | collagen, type XI, alpha 2 | 3.37E−03 | 0.93 | 2.24 |
| SELL | PRO | P14151 | selectin L | 3.41E−03 | 4.98 | 7.56 |
| ENPP2 | PRO | Q13822 | ectonucleotide | 3.59E−03 | 1.5 | 3.99 |
| | | | pyrophosphatase/ | | | |
| | | | phosphodiesterase 2 | | | |
| GP5LA | GLY | FA2[6]G1 | | 3.62E−03 | 0.06 | 0.08 |
| GP19LA | GLY | FA2BG2S | | 3.73E−03 | 0.17 | 0.21 |
| | | [3]1 | | | | |
| | | FA2BG2S | | | | |
| | | [6]1 | | | | |
| CD80 | GE | 941 | CD80 (costimulatory | 3.78E−03 | −9.17 | −8.24 |
| | | | molecule) | | | |
| F2 | PRO | P00734 | coagulation factor II | 3.80E−03 | 88.77 | 118.4 |
| | | | (thrombin) | | | |
| ITIH4 | PRO | Q14624 | inter-alpha-trypsin | 3.99E−03 | 396.21 | 362.86 |
| | | | inhibitor heavy chain | | | |
| | | | family, member 4 | | | |
| ACTBL2 | PRO | Q562R1 | actin, beta-like 2 | 4.08E−03 | 3.78 | 2.58 |
| ITIH4 | PRO | B7ZKJ8 | inter-alpha-trypsin | 4.08E−03 | 396.42 | 362.46 |
| | | | inhibitor heavy chain | | | |
| | | | family, member 4 | | | |
| KLRG1 | GE | 10219 | killer cell lectin-like | 4.11E−03 | −3.67 | −2.9 |
| | | | receptor subfamily G, | | | |
| | | | member 1 | | | |
| ITIH4 | PRO | E9PGN5 | inter-alpha-trypsin | 4.17E−03 | 395.42 | 362.13 |
| | | | inhibitor heavy chain | | | |
| | | | family, member 4 | | | |
| HBE1 | PRO | P02100 | hemoglobin, epsilon 1 | 4.51E−03 | 11.98 | 1.25 |
| IgA | ELISA | NA | IgA | 4.58E−03 | 1.11 | 0.41 |
| GP6LA | GLY | FA2[3]G1 | | 4.79E−03 | 0.01 | 0.03 |
| GP2HA | GLY | FA2 | | 4.85E−03 | 2.98 | 4.99 |
| APOD | PRO | P05090 | apolipoprotein D | 5.18E−03 | 0.94 | 1.89 |
| CRY2 | PRO | Q49AN0 | cryptochrome 2 | 5.32E−03 | 2.37 | 1.18 |
| PLG | PRO | P00747 | plasminogen | 5.42E−03 | 57.53 | 72.3 |
| PPP6R2 | PRO | B3KMJ7 | protein phosphatase 6, | 5.45E−03 | 4.28 | 0.65 |
| | | | regulatory subunit 2 | | | |
| DOCK3 | PRO | Q8IZD9 | dedicator of cytokinesis 3 | 5.47E−03 | 2.6 | 0.84 |
| FCGR2A | GE | 2212 | Fc fragment of IgG, low | 5.73E−03 | 2.06 | 0.48 |
| | | | affinity IIa, receptor | | | |
| | | | (CD32) | | | |
| CCR2 | GE | 729230 | chemokine (C-C motif) | 6.66E−03 | −2.4 | −3.4 |
| | | | receptor 2 | | | |
| MASP1 | PRO | P48740 | mannan-binding lectin | 6.69E−03 | 7.73 | 12.25 |
| | | | serine peptidase 1 | | | |
| | | | (C4/C2 activating | | | |
| | | | component of Ra- | | | |
| | | | reactive factor) | | | |
| GP31HA | GLY | A3G3S[3,3, | | 6.74E−03 | 0.42 | 0.26 |
| | | 3]3 | | | | |
| ACTB | GE | 60 | actin, beta | 7.45E−03 | 4.74 | 4.15 |
| CTSD | PRO | P07339 | cathepsin D | 7.61E−03 | 4.15 | 0.83 |
| TNF | GE | 7124 | tumor necrosis factor | 7.65E−03 | −4.82 | −5.53 |
| CEP290 | PRO | O15078 | centrosomal protein | 7.71E−03 | 3.24 | 1.43 |
| | | | 290 kDa | | | |
| IGFBP3 | PRO | P17936 | insulin-like growth | 8.01E−03 | 2.06 | 3.55 |
| | | | factor binding protein 3 | | | |
| DHRS11 | PRO | A8MXC2 | dehydrogenase/reductase | 8.04E−03 | 0.67 | 2.07 |
| | | | (SDR family) | | | |
| | | | member 11 | | | |
| QSOX1 | PRO | O00391 | quiescin Q6 sulfhydryl | 8.16E−03 | 3.04 | 4.86 |
| | | | oxidase 1 | | | |
| MASP1 | PRO | F8W876 | mannan-binding lectin | 8.22E−03 | 5.31 | 8.56 |
| | | | serine peptidase 1 | | | |
| | | | (C4/C2 activating | | | |
| | | | component of Ra- | | | |
| | | | reactive factor) | | | |
| IgA_G1_S1 | IgGLY | A2,G1,S1 | | 8.36E−03 | 8.79 | 2.12 |
| GP20HA | GLY | A2G2S[3,6]2 | | 8.60E−03 | 3.53 | 4.55 |

Analytic annotations:
PRO; shot-gun proteomics,
GE; gene expression,
GLY; plasma glycomics,
IgGLY; site-specific Ig glycosylation.
Glycan annotations according to Oxford Symbol nomenclature.
All N-glycans have two core GlcNAcs;
F at the start of the abbreviation indicates a core α(1-6)fucose linked to inner GlcNAc;
Mx, number (x) of mannose on core GlcNAcs;
Ax, number of antenna (GlcNAc) on trimannosyl core;
A2, biantennary with both GlcNAcs as β(1-2) linked;
A3, triantennary with a GlcNAc linked β(1-2) to both mannose and a third GlcNAc linked β(1,4) to the α(1-3) linked mannose;
A3′; triantennary with a GlcNAc linked β(1-2) to both mannose and the third GlcNAc linked β(1-6) mannose;
B, bisecting GlcNAc linked β(1-4) to β(1-3) mannose;
Gx, number (x) of β1-4 linked galasose on the antenna;
Fx, number (x) of linked fucose on antenna, (4) or (3) after the F indicates that the Fuc is α(1-4) or α(1-3) linked to the GlcNAc;
Sx, number (x) sialic acids linked to galactose; the number 3 or 6 in parenthesis after S indicates whether the sialic acid is in an α(2-3) or α(2-6) linkage.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth.

All publications, patents and patent applications are herein incorporated by reference in their entirety to the same extent as if each individual publication, patent, or patent application was specifically and individually indicated to be incorporated by reference in its entirety.

What is claimed is:

1. A method for treating Kawasaki disease in a subject, said method comprising:
(a) selecting a subject having a fever and one or more of: red eyes; a red swollen tongue; red skin on the palms of the hands and/or soles of the feet; peeling of the skin on the hands and/or feet; a rash on the main part of the body and/or in the genital area; and swollen lymph nodes;
(b) obtaining a biological sample from said subject;
(c) determining the level of one or more proteins from Table 1 and/or Table 2 in said biological sample;
(d) comparing the level of said one or more proteins to a predetermined control value; and
(e) administering a Kawasaki disease therapy to said subject if the level of said one or more proteins is indicative that said subject may benefit from a Kawasaki disease therapy;
wherein an increased level of at least one protein of Table 1, as compared to a reference, and/or a decreased level of at least one protein of Table 2, as compared to a reference, is indicative that the subject may benefit from a Kawasaki disease therapy.

2. A method for treating Kawasaki disease in a subject, said method comprising:
(a) selecting a subject having a fever and one or more of: red eyes; a red swollen tongue; red skin on the palms of the hands and/or soles of the feet; peeling of the skin on the hands and/or feet; a rash on the main part of the body and/or in the genital area; and swollen lymph nodes;
(b) administering a Kawasaki disease therapy to said subject;
(c) obtaining a biological sample from said subject;
(d) determining the level of one or more proteins from Table 6, Table 7, Table 8, and/or Table 9, one or more mRNAs from Table 10, Table 11, Table 12, and/or Table 13, and/or one or more glycans of Table 14, Table 15, Table 16, and/or Table 17, in said biological sample;
(e) comparing the level of said one or more proteins to a predetermined control value; and
(f) administering a Kawasaki disease therapy, other than, or in addition to IVIG, to said subject if the level of said one or more proteins is indicative that said subject may benefit from a Kawasaki disease therapy other than, or in addition to, IVIG;
wherein an increased level of at least one protein of Table 6 and/or Table 8, at least one mRNA of Table 10 and/or 12, and/or at least one glycan of Table 14 and/or 16, as compared to a reference, and/or a decreased level of at least one protein of Table 7 and/or Table 9, at least one mRNA of Table 11 and/or Table 13, and/or at least one glycan of Table 15 and/or Table 17, as compared to a reference, is indicative that the subject may benefit from a Kawasaki disease therapy other than, or in addition to, IVIG.

3. A method for treating Kawasaki disease in a subject, said method comprising:
(a) selecting a subject having a fever and one or more of: red eyes; a red swollen tongue; red skin on the palms of the hands and/or soles of the feet; peeling of the skin on the hands and/or feet; a rash on the main part of the body and/or in the genital area; and swollen lymph nodes;
(b) obtaining a biological sample from said subject;
(c) determining the binding of IgG in said biological sample to one or more proteins comprising an amino acid sequence of any one of SEQ ID NOs: 1 to 68;
(d) comparing the binding of IgG to said one or more proteins to a predetermined control value; and
(e) administering a Kawasaki disease therapy to said subject if the binding of IgG to said one or more proteins is indicative that said subject may benefit from a Kawasaki disease therapy;
wherein increased binding of IgG to at least one protein comprising an amino acid sequence of any one of SEQ ID NOs:1 to 68, as compared to a reference, is indicative that the subject may benefit from a Kawasaki disease therapy.

4. A method for treating Kawasaki disease in a subject, said method comprising:
(a) determining the level of one or more proteins from Table 1 and/or Table 2 in a biological sample obtained from said subject; and
(b) administering a Kawasaki disease therapy to said subject if the level of said one or more proteins is indicative that said subject may benefit from a Kawasaki disease therapy;
wherein an increased level of at least one protein of Table 1, as compared to a reference, and/or a decreased level of at least one protein of Table 2, as compared to a reference, is indicative that the subject may benefit from a Kawasaki disease therapy.

5. A method for treating Kawasaki disease in a subject, said method comprising:
(a) administering a Kawasaki disease therapy to said subject;
(b) determining the level of one or more proteins from Table 6, Table 7, Table 8, and/or Table 9, one or more mRNAs from Table 10, Table 11, Table 12, and/or Table 13, and/or one or more glycans from Table 14, Table 15, Table 16, and/or Table 17, in a biological sample obtained from said subject; and
(c) administering a Kawasaki disease therapy, other than, or in addition to, IVIG to said subject if the level of said one or more proteins is indicative that said subject may benefit from a Kawasaki disease therapy other than, or in addition to, IVIG;
wherein an increased level of at least one protein of Table 6 and/or Table 8, at least one mRNA of Table 10 and/or 12, and/or at least one glycan of Table 14 and/or Table 16, as compared to a reference, and/or a decreased level of at least one protein of Table 7 and/or Table 9, at least one mRNA of Table 11 and/or Table 13, and/or at least one glycan of Table 15 and/or Table 17, as compared to a reference, is indicative that the subject may benefit from a Kawasaki disease therapy.

6. A method for treating Kawasaki disease in a subject, said method comprising:
(a) the binding of IgG in said biological sample to one or more proteins comprising an amino acid sequence of any one of SEQ ID NOs: 1 to 68; and
(b) administering a Kawasaki disease therapy to said subject if the binding of IgG to said one or more proteins is indicative that said subject may benefit from a Kawasaki disease therapy;
wherein increased binding of IgG to at least one protein comprising an amino acid sequence of any one of SEQ ID NOs:1 to 68, as compared to a reference, is indicative that the subject may benefit from a Kawasaki disease therapy.

7. The method of any one of claims 1 to 6, further comprising:
determining the level of one or more proteins from Table 4 and/or Table 5 in a biological sample obtained from said subject; and
administering a therapy other than, or in addition to, IVIG therapy if the level of said one or more proteins is indicative that said subject may benefit from a Kawasaki disease therapy other than, or in addition to, IVIG therapy;
wherein an increased level of at least one protein of Table 4, as compared to a reference and/or a decreased level of at least one protein of Table 5, as compared to a reference, is indicative that the subject may benefit from a Kawasaki disease therapy, other than or in addition to, IVIG therapy.

8. The method of any one of claims 1 to 7, wherein said one or more proteins comprises complement factor H, collagen type 1 alpha 1, gelsolin, alpha-1-antichymotrypsin, and sex hormone binding globulin.

9. A method of treating Kawasaki disease in a subject, said method comprising:
(a) determining the level of one or more proteins from Table 4, Table 5, Table 6, Table 7, Table 8, and/or Table 9, one or more mRNAs of Table 10, Table 11, Table 12, and/or Table 13, and/or one or more glycans of Table 14, Table 15, Table 16, and/or Table 17 in a biological sample obtained from said subject; and
(b) administering a therapy other than, or in addition to, IVIG therapy if the level of said one or more proteins is indicative that said subject may benefit from a Kawasaki disease therapy other than, or in addition to, IVIG therapy;
wherein an increased level of at least one protein of Table 4, Table 6, and/or Table 8, at least one mRNA of Table 10 or Table 12, and/or at least one glycan of Table 14 or Table 16, as compared to a reference, and/or a decreased level of at least one protein of Table 5, Table 7, and/or Table 9, at least one mRNA of Table 11 or Table 13, and/or at least one glycan of Table 15 or Table 17, as compared to a reference, is indicative that the subject may benefit from a Kawasaki disease therapy other than, or in addition to, IVIG therapy.

10. The method of claim 1 or 4, wherein said one or more proteins from Table 1 and/or Table 2 comprises two or more proteins from Table 1 and/or Table 2.

11. The method of claim 1 or 4, wherein said one or more proteins from Table 1 and/or Table 2 comprises three or more proteins from Table 1 and/or Table 2.

12. The method of any one of claims 7 to 9, wherein said one or more proteins from Table 4 and/or Table 5 comprises two or more proteins from Table 4 and/or Table 5.

13. The method of any one of claims 7 to 9, wherein said one or more proteins from Table 4 and/or Table 5 comprises three or more proteins from Table 4 and/or Table 5.

14. The method of any one of claims 1 to 13, further comprising contacting the biological sample with one or more binding agents capable of specifically binding to the one or more proteins.

15. The method of any one of claims 1 to 14, wherein said Kawasaki disease therapy comprises administration of IVIG to said subject.

16. The method of any one of claims 1 to 15, wherein said Kawasaki disease therapy comprises administration of an anticoagulant to said subject.

17. The method of claim 16, wherein said anticoagulant is enoxaparin or a pharmaceutically acceptable salt thereof.

18. The method of any one of claims 4 to 17, wherein said subject has one or more of: a fever; red eyes; a rash on the main part of the body and/or in the genital area; red, dry, cracked lips; a red, swollen tongue; swollen, red skin on the palms of the hands and/or soles of the feet; swollen lymph nodes; irritability; peeling of the skin on the hands and/or feet; joint pain; diarrhea; vomiting; and abdominal pain.

19. The method of claim 18, wherein said subject has a fever and one or more of: red eyes; a red swollen tongue; red skin on the palms of the hands and/or soles of the feet; peeling of the skin on the hands and/or feet; a rash on the main part of the body and/or in the genital area; and swollen lymph nodes.

20. The method of claim 19, wherein said fever lasts for more than four days.

21. The method of any one of claims 1 to 20, wherein said subject exhibits the clinical symptoms of cardiac artery aneurysms and/or stenosis of the arteries.

22. The method of any one of claims 1 to 21, wherein said subject has not been diagnosed with cardiac artery aneurysms and/or stenosis of the arteries prior to determining the level of said one or more proteins.

23. The method of any one of claims 1 to 22, wherein a biological sample is obtained from said subject prior to the commencement of IVIG therapy.

24. The method of any one of claims 1 to 22, wherein a biological sample is obtained from said subject after commencement of IVIG therapy.

25. The method of claim 24, wherein said biological sample is obtained from said subject within 24 hours after commencement of IVIG therapy.

26. The method of any one of claims 1 to 25, wherein said biological sample is a tissue sample, whole blood, plasma, urine, saliva, pancreatic juice, bile, or serum sample.

27. The method of claim 26, wherein said biological sample is a plasma sample.

28. The method of any one of claims 1 to 27, further comprising processing said biological sample prior to determining the level of said one or more said proteins.

29. The method of claim 28, wherein processing said biological sample comprises fractionation of said biological sample.

30. The method of claim 29, wherein said fractionation comprises the removal of proteins present at concentrations greater than 0.01 g/dL.

31. The method of any one of claims 1 to 30, wherein said level of said one or more proteins is determined by a hybridization assay, an immunoassay, a mass spectrometric assay, a single molecule detection assay, and/or a fluorescence in situ hybridization assay.

32. The method of claim 31, wherein said level of said one or more proteins is determined by an immunoassay.

33. The method of claim 32, wherein said immunoassay is an ELISA assay.

34. The method of any one of claims 1 to 33, wherein said subject is less than 13 years old.

35. The method of claim 34, wherein said subject is less than 5 years old.

36. The method of any one of claims 1 to 35, wherein said subject is Asian or Afro-Caribbean.

37. The method of claim 36, wherein said subject is Asian.

38. The method of claim 37, wherein said subject is Japanese or Korean.

39. The method of any one of claims 1 to 38, wherein said level of said one or more proteins and/or binding of IgG in said sample is determined at least twice within 365 days.

40. A kit for selecting a subject that may benefit from a Kawasaki disease therapy, said kit comprising a set of two or more binding agents, each of said binding agents being capable of specifically binding to at least one protein from Table 1 and/or Table 2, and/or one or more proteins comprising an amino acid sequence of any one of SEQ ID NOs:1 to 68.

41. The kit of claim 40, wherein said set comprises binding agents capable of specifically binding to complement factor H, collagen type 1 alpha 1, gelsolin, alpha-1-antichymotrypsin, and sex hormone binding globulin.

42. The kit of claim 40 or 41, further comprising a set of one or more binding agents, each of which is capable of specifically binding to at least one protein from Table 4, Table 5, Table 6, Table 7, Table 8, and/or Table 9, at least one mRNA from Table 10, Table 11, Table 12, and/or Table 13, and/or at least one glycan from Table 14, Table 15, Table 16, and/or Table 17.

43. A kit for selecting a subject that may benefit from a Kawasaki disease therapy other than, or in addition to, IVIG therapy, said kit comprising a set of two or more binding agents, each of said binding agents being capable of specifically binding to at least one protein from Table 4, Table 5, Table 6, Table 7, Table 8, and/or Table 9, at least one mRNA from Table 10, Table 11, Table 12, and/or Table 13, and/or at least one glycan from Table 14, Table 15, Table 16, and/or Table 17.

44. The kit of any one of claims 40 to 43 further comprising instructions for use of said kit to determine the level of proteins in a biological sample.

45. The kit of any one of claims 40 to 44, wherein said binding agents are provided on a solid support.

46. The kit of any one of claims 40 to 45, wherein said binding agents are antibodies.

47. A method for diagnosing Kawasaki disease in a subject, said method comprising determining the level of one or more proteins in a biological sample obtained from said subject and/or the binding of IgG in said sample to one or more proteins comprising an amino acid sequence of any one of SEQ ID NOs:1 to 68, with a kit of any one of claims 40 to 42, wherein an increased level of at least one protein of Table 1, as compared to a reference, and/or a decreased level of at least one protein of Table 2, as compared to a reference, and/or increased binding of IgG to at least one protein comprising an amino acid sequence of any one of SEQ ID NOs:1 to 68 is indicative of said subject having Kawasaki disease.

48. A method for diagnosing a predisposition to develop cardiac artery aneurysms or stenosis in a subject, said method comprising determining the level of one or more proteins in a biological sample obtained from said subject with a kit of claim 42 or 43, wherein an increased level of at least one protein of Table 4, Table 6, and/or Table 8, at least one mRNA of Table 10 or Table 12, and/or at least one glycan of Table 14 or Table 16, as compared to a reference, and/or a decreased level of at least one protein of Table 5, Table 7, and/or Table 9, at least one mRNA of Table 11 or Table 13, and/or at least one glycan of Table 15 or Table 17, as compared to a reference, is indicative of said subject having a predisposition to develop cardiac artery aneurysms or stenosis.

49. A method for classifying a subject as one who may benefit from a Kawasaki disease therapy, said method comprising determining the level of one or more proteins in a biological sample obtained from said subject and/or the binding of IgG in said sample to one or more proteins comprising an amino acid sequence of any one of SEQ ID NOs:1 to 68 with a kit of any one of claims 40 to 42, wherein said subject is classified based on an increased level of at least one protein of Table 1, as compared to a reference, and/or a decreased level of at least one protein of Table 2, as compared to a reference, and/or increased binding of IgG in said sample to at least one protein comprising an amino acid sequence of any one of SEQ ID NOs:1 to 68.

50. A method of classifying a subject as one who may benefit from a Kawasaki disease therapy other than, or in addition to, IVIG therapy, said method comprising determining the level of one or more proteins in a biological sample obtained from said subject with a kit of claim 42 or 43, wherein said subject is classified based on an increased level of at least one protein of Table 4, Table 6, and/or Table 8, at least one mRNA of Table 10 or Table 12, and/or at least one glycan of Table 14 or Table 16, as compared to a reference, and/or a decreased level of at least one protein of Table 5, Table 7, and/or Table 9, at least one m RNA of Table 11 or Table 13, and/or at least one glycan of Table 15 or Table 17, as compared to a reference.

51. A method for treating Kawasaki disease in a subject, said method comprising:
(a) determining the level of one or more proteins in a biological sample obtained from said subject and/or binding of IgG in said sample to one or more proteins comprising an amino acid sequence of any one of SEQ ID NOs:1 to 68 with a kit of any one of claims 40 to 42;
(b) administering a Kawasaki disease therapy if the level of said one or more proteins and/or binding of IgG in said sample is indicative that said subject may benefit from a Kawasaki disease therapy;
wherein an increased level of at least one protein of Table 1, as compared to a reference, and/or a decreased level of at least one protein of Table 2, as compared to a reference, and/or increased binding of IgG in said sample to at least one protein comprising an amino acid sequence of any one of SEQ ID NOs:1 to 68, is indicative that said subject may benefit from a Kawasaki disease therapy.

52. A method for treating Kawasaki disease in a subject, said method comprising:
(a) determining the level of one or more proteins in a biological sample obtained from said subject with a kit of claim 42 or 43;
(b) administering a therapy other than, or in addition to, IVIG therapy if the level of said one or more proteins is indicative that said subject may benefit from a Kawasaki disease therapy other than, or in addition to, IVIG therapy;
wherein an increased level of at least one protein of Table 4, Table 6, and/or Table 8, at least one mRNA of Table 10 or Table 12, and/or at least one glycan of Table 14 or Table 16, as compared to a reference, and/or a decreased level of at least one protein of Table 5, Table 7, and/or Table 9, at least one mRNA of Table 11 or Table 13, and/or at least one glycan of Table 15 or Table 17, as compared to a reference, is indicative that the subject may benefit from a Kawasaki disease therapy other than, or in addition to, IVIG therapy.
